(19) United States
(12) Patent Application Publication
(10) Pub. No.: US 2006/0270841 A1
(43) Pub. Date: Nov. 30, 2006

(54) Assays to monitor amyloid precursor protein processing

(76) Inventors: Amy S. Espeseth, Chalfont, PA (US); Marc Ferrer, Madia, PA (US); Osvaldo A. Flores, North Wales, PA (US); Daria J. Hazuda, Doylestown, PA (US); James Inglese, Lansdale, PA (US); Michael D. Miller, Chalfont, PA (US); Bruce Register, Berwyn, PA (US); Xiao-Ping Shi, Warrington, PA (US); Adam J. Simon, Lanhorne, PA (US); Paul D. Zuck, Trenton, NJ (US)

Correspondence Address: MERCK AND CO., INC, P O BOX 2000, RAHWAY, NJ, 07065-0907, US

(21) Appl. No.: 10/504,446

(22) PCT Filed: Feb. 25, 2003
(86) PCT No.: PCT/US03/05458

§ 371 Date: Aug. 12, 2004

Related U.S. Application Data
(60) Provisional application No. 60/360,274, filed on Feb. 27, 2002.

(51) Int. Cl. C12Q 1/68; C07H 21/04; C12P 21/06; C12N 9/64

(52) U.S. Cl. 536/23.2; 435/6; 435/69.1; 435/226; 435/320.1; 435/325

(57) ABSTRACT

The present invention provides DNA constructs, genetically engineered host cells, and methods for identifying inhibitors of amyloid precursor protein (APP) processing. The methods provide for the convenient identification, in a single assay, of inhibitors of β-secretase and γ-secretase as well as other forms of APP processing. The methods rely on fusion proteins of APP and transcription factors in which APP processing releases the transcription factors, allowing the transcription factors to activate transcription of a reporter gene. Inhibitors are identified as substances that block or diminish transcription factor release from the fusion protein, thereby causing a diminution of reporter gene readout.

### CROSS-REFERENCE TO RELATED APPLICATIONS

This application claims the benefit of U.S. Provisional Application No. 60/360,274, filed Feb. 27, 2002, the contents of which are incorporated herein by reference in their entirety.

### STATEMENT REGARDING FEDERALLY-SPONSORED R&D

Not applicable.

### REFERENCE TO MICROFICHE APPENDIX

Not applicable.

### FIELD OF THE INVENTION

The present invention is directed to the field of Alzheimer's disease. In particular, the present invention provides novel methods of identifying substances that are specific inhibitors of various steps in the processing of amyloid precursor protein.

### BACKGROUND OF THE INVENTION

Alzheimer's disease is a common, chronic neurodegenerative disease, characterized by a progressive loss of memory and sometimes severe behavioral abnormalities, as well as an impairment of other cognitive functions that often leads to dementia and death. It ranks as the fourth leading cause of death in industrialized societies after heart disease, cancer, and stroke. The incidence of Alzheimer's disease is high, with an estimated 2.5 to 4 million patients affected in the United States and perhaps 17 to 25 million worldwide. Moreover, the number of sufferers is expected to grow as the population ages.

A characteristic feature of Alzheimer's disease is the presence of large numbers of insoluble deposits, known as amyloid plaques, in the brains of those affected. Autopsies have shown that amyloid plaques are found in the brains of virtually all Alzheimer's patients and that the degree of amyloid plaque deposition correlates with the degree of dementia (Cummings & Cotman, 1995, Lancet 326:1524-1587). While some opinion holds that amyloid plaques are a late stage by-product of the disease process, the consensus view is that amyloid plaques are more likely to be intimately, and perhaps causally, involved in Alzheimer's disease.

A variety of experimental evidence supports this view. For example, Aβ, a primary component of amyloid plaques, is toxic to neurons in culture and transgenic mice that overproduce Aβ in their brains show significant deposition of Aβ into amyloid plaques as well as significant neuronal toxicity (Yankner, 1990, Science 250:279-282; Mattson et al., 1992, J. Neurosci. 12:379-389; Games et al., 1995, Nature 373:523-527; LaFerla et al., 1995, Nature Genetics 9:21-29). Mutations in the APP gene, leading to increased Aβ production, have been linked to heritable forms of Alzheimer's disease (Goate et al., 1991, Nature 349:704-706; Chartier-Harlan et al., 1991, Nature 353:844-846; Murrel et al., 1991,Science 254:97-99; Mullan et al., 1992, Nature Genetics 1:345-347). Presenilin-1 (PS 1) and presenilin-2 (PS2) related familial early-onset Alzheimer's disease (FAD) shows disproportionately increased production of Aβ1-42, the 42 amino acid isoform of Aβ, as opposed to Aβ1-40, the 40 amino acid isoform (Scheuner et al, 1996, Nature Medicine 2:864-870). The longer isoform of Aβ is more prone to aggregation than the shorter isoform (Jarrett et al, 1993, Biochemistry 32:4693-4697). Injection of the insoluble, fibrillar form of Aβ into monkey brains results in the development of pathology (neuronal destruction, tau phosphorylation, microglial proliferation) that closely mimics Alzheimer's disease in humans (Geula et al., 1998, Nature Medicine 4:827-831). See Selkoe, 1994, J. Neuropathol. Exp. Neurol. 53:438-447 for a review of the evidence that amyloid plaques have a central role in Alzheimer's disease.

Aβ, a 39-43 amino acid peptide derived by proteolytic cleavage of the amyloid precursor protein (APP), is the major component of amyloid plaques (Glenner & Wong, 1984, Biochem. Biophys. Res. Comm. 120:885-890). APP is actually a family of polypeptides produced by alternative splicing from a single gene. Major forms of APP are known as APP₆₉₅, APP₇₅₁, and APP₇₇₀, with the subscripts referring to the number of amino acids in each splice variant (Ponte et al., 1988, Nature 331:525-527; Tanzi et al., 1988, Nature 331:528-530; Kitaguchi et al., 1988, Nature 331:530-532). APP is membrane bound and undergoes proteolytic cleavage by at least two pathways. In one pathway, cleavage by an enzyme known as α-secretase occurs while APP is still in the trans-Golgi secretory compartment (Kuentzel et al., 1993, Biochem. J. 295:367-378). This cleavage by α-secretase occurs within the Aβ portion of APP, thus precluding the formation of Aβ. In another proteolytic pathway, cleavage of the Met₅₉₆-Asp₅₉₇ bond (numbered according to the 695 amino acid protein) by an enzyme known as β-secretase occurs. This cleavage by β-secretase generates the N-terminus of Aβ. The C-terminus is formed by cleavage by a second enzyme known as γ-secretase. The C-terminus is actually a heterogeneous collection of cleavage sites rather than a single site since γ-secretase activity occurs over a short stretch of APP amino acids rather than at a single peptide bond. Peptides of 40 or 42 amino acids in length (Aβ1-40 and Aβ11-42, respectively) predominate among the C-termini generated by γ-secretase. Aβ1-42 is more prone to aggregation than Aβ1-40, is the major component of amyloid plaque (Jarrett et al., 1993, Biochemistry 32:4693-4697; Kuo et al., 1996, J. Biol. Chem. 271:4077-4081), and its production is closely associated with the development of Alzheimer's disease (Sinha & Lieberburg, 1999, Proc. Natl. Acad. Sci. USA 96:11049-11053). The bond cleaved by γ-secretase appears to be situated within the transmembrane domain of APP. It is unclear as to whether the C-termini of Aβ1-40 and Aβ1-42 are generated by a single γ-secretase protease with sloppy specificity or by two distinct proteases. For a review that discusses APP and its processing, see Selkoe, 1998, Trends Cell. Biol. 8:447-453.

Much interest has focused on the possibility of inhibiting the development of amyloid plaques as a means of preventing or ameliorating the symptoms of Alzheimer's disease. To that end, a promising strategy is to inhibit the activity of β- and γ-secretase, the two enzymes that together are responsible for producing Aβ. This strategy is attractive because, if the formation of amyloid plaques as a result of the deposition of Aβ is a cause of Alzheimer's disease, inhibiting the activity of one or both of the two secretases would intervene in the disease process at an early stage, before late-stage events such as inflammation or apoptosis occur. Such early stage intervention is expected to be particularly beneficial (see, e.g., Citron, 2000, Molecular Medicine Today 6:392-397).

To that end, various assays have been developed that are directed to the identification of compounds that may interfere with the production of Aβ or its deposition into amyloid plaques. U.S. Pat. No. 5,441,870 is directed to methods of monitoring the processing of APP by detecting the production of amino terminal fragments of APP. U.S. Pat. No. 5,605,811 is directed to methods of identifying inhibitors of the production of amino terminal fragments of APP. U.S. Pat. No. 5,593,846 is directed to methods of detecting soluble Aβ by the use of binding substances such as antibodies. Esler et al., 1997, Nature Biotechnology 15:258-263 described an assay that monitored the deposition of Aβ from solution onto a synthetic analogue of an amyloid plaque. The assay was suitable for identifying compounds that could inhibit the deposition of Aβ. However, this assay is not suitable for identifying substances, such as inhibitors of β- or γ-secretase, that would prevent the formation of Aβ.

Various groups have cloned and sequenced cDNA encoding a protein that is believed to be β-secretase (Vassar et al., 1999, Science 286:735-741; Hussain et al., 1999, Mol. Cell. Neurosci. 14:419-427; Yan et al., 1999, Nature 402:533-537; Sinha et al., 1999, Nature 402:537-540; Lin et al., 2000, Proc. Natl. Acad. Sci. USA 97:1456-1460) but the identity of γ-secretase has been more elusive. A pair of proteins known as presenilin-1 and presenilin-2 are viewed as possible candidates (Selkoe & Wolfe, 2000, Proc. Natl. Acad. Sci. USA 97:5690-5692).

Presenilin-1 (PS1) and presenilin-2 (PS2) are polytopic membrane proteins that are involved in γ-secretase-mediated processing of APP. The most common cause of familial early-onset Alzheimer's disease is the autosomal dominant inheritance of assorted mutations in the PS1 gene (Sherrington et al., 1995, Nature 375:754-760). These PS1 mutations lead to increased production of Aβ1-42 (Scheuner et al., 1996, Nature Medicine 2:864-870; Duff et al., 1996, Nature 383:710-713; Borchelt et al., 1996, Neuron 17:1005-1013). Similarly, certain mutations in PS2 cause familial early-onset Alzheimer's disease and increased generation of Aβ42 (Levy-Lahad et al., 1995, Science 269:970-973). Cultured isolated neurons from PS1-deficient mice exhibit reduced γ-secretase-mediated cleavage of APP (De Strooper et al., 1998, Nature 391:387-390). It was suggested that PS1 might influence trafficking of APP and/or γ-secretase or it might play a more direct role in proteolytic cleavage of APP. Directed mutagenesis of two conserved transmembrane-situated aspartates in PS1 was shown to inactivate γ-secretase activity in cellular assays, suggesting that PS1 is either a required diaspartyl cofactor for γ-secretase or is itself γ-secretase (Wolfe et al., 1999, Nature 398:513-517). Moreover, Li et al., 2000, Nature 405:689-694 made photoactivatable derivatives of a highly specific and potent aspartyl protease transition state analog inhibitor and found that the inhibitor selectively labeled presenilin fragments.

Co-immunoprecipitation experiments have shown that PS1 and PS2 interact directly with the immature forms of APP in the endoplasmic reticulum where the disease-associated amyloid Aβ1-42 peptide is probably generated (Xia et al., 1997 Proc. Natl. Acad. Sci. USA 94:8208-8213; Weidemann et al., 1997, Nat. Med. 3:328-332). Knock-out of PS1 activity greatly diminishes γ-secretase cleavage of APP (De Strooper et al., 1998, Nature 391:387-390). PS1 knock-outs do not exhibit total lack of γ-secretase activity but knock-out of both PS1 and PS2 activity does result in a total loss of γ-secretase activity (Herreman et al., 2000, Nat. Cell. Biol. 2:461-462; Zhang et al., 2000, Nat. Cell Biol. 2:463-465), suggesting that PS2 has a similar function to PS1 in the processing of APP.

Karlström et al., (Journal of Biological Chemistry papers in press, published on Dec. 13, 2001 as Manuscript C100649200) describes an assay designed specifically to identify inhibitors of γ-secretase cleavage of APP. The authors inserted the GAL4 DNA binding domain fused to the VP16 transactivation domain into C99, a portion of APP containing the 99 carboxy-terminal amino acids. This fragment of APP contains the γ-secretase cleavage site but lacks the β-secretase cleavage site. Transaction of a UAS reporter plasmid by GAL4-VP16 confirmed cleavage of the Gal4-VP16/C99 substrate by γ-secretase only. Thus, the assay is capable of detecting γ-secretase inhibitors but not inhibitors of β-secretase or other modulators of APP processing requiring the N-terminal domain of APP.

Cao & Stühoff, 2001, Science 293:115-120 described work in which the GAL4 and LexA DNA binding domains were inserted into APP to demonstrate the potential of the cleaved C-terminus of APP for transcriptional co-activation. In this article, a transcriptional factor was not fused to APP and no attempt was made to develop an assay for the identification of APP processing inhibitors.

Sisodia, 1992, Proc. Natl. Acad. Sci. USA 89:6975-6979 described various changes in the amino acid sequence of APP in the region of the α-secretase cleavage site and the effect of those changes on cleavage by α-secretase. A change of K to V at position 612 of the 695 amino acid version of APP led to reduced cleavage by α-secretase.

U.S. Pat. No. 6,333,167 B1 discloses an assay involving DNA constructs encoding portions of membrane proteins containing sites that are susceptible to cleavage by proteases that are fused to transcriptional repressors. Such constructs are introduced into cells that contain a reporter gene under the control of a promoter that is sensitive to the repressor. In the absence of an inhibitor of the protease, the fusion protein is cleaved by the protease, releasing a membrane protein/repressor fusion protein that translocates to the nucleus and represses transcription from the reporter gene. In the presence of an inhibitor of the protease, the membrane protein/repressor fusion protein is not released and thus cannot repress transcription from the reporter. An increase in reporter expression can therefore be used as a readout for the presence of an inhibitor.

### SUMMARY OF THE INVENTION

The present invention is directed to methods of identifying inhibitors of the processing of amyloid precursor protein (APP) that are capable of identifying inhibitors of a number of steps of such processing. Unlike prior methods, the methods of the present invention can be used to screen for inhibitors of β-secretase cleavage, γ-secretase cleavage, APP extracellular signaling, or APP cytoplasmic signaling in a single assay.

The methods employ a recombinant eukaryotic cell that is capable of processing APP. The cell has been engineered to express a fusion protein that contains amino acid sequences encompassing both the β-secretase cleavage site of APP and the γ-secretase cleavage site. The fusion protein also contains a transcription factor fused in frame to the APP sequences.

When the recombinant cell is further engineered to contain a reporter gene, in which transcription of the reporter gene is driven by a regulatory DNA sequence that is inactive in the absence of the transcription factor but active in the transcription factor's presence, a system useful for screening for APP processing inhibitors is provided. Since the recombinant cell has been selected so as to be capable of processing APP, the fusion protein will be processed, releasing the transcription factor and activating transcription of the reporter gene. The reporter gene has been preselected so that activation of the reporter gene leads to a detectable phenotype.

The system is utilized by exposing the recombinant cell to substances that are to be tested for the ability to inhibit APP processing. Those substances that are actually inhibitors of APP processing will cause diminished processing of the fusion protein, leading to smaller amounts of the transcription factor being released. This leads to less transcription of the reporter gene. This results in a decrease in the phenotypic effect of the reporter gene that can be observed.

### BRIEF DESCRIPTION OF THE DRAWINGS

FIG. 1A-G shows a schematic diagram of several APP/transcription factor fusion constructs.

FIG. 2A-B shows the DNA sequence (SEQ ID NO:1) of the fusion protein APP(1-651)SW, K612V-TATexonI(M1L) APP (664-695).

FIG. 3 shows the amino acid sequence (SEQ ID NO:2) of the fusion protein APP(1-651)SW, K612V-TATexonI(M1L) APP (664-695) with the various different regions of the fusion protein demarcated. 1=amino acids 1-651 of APP; 2=region of β-secretase cleavage; 3=K612V mutation; 4=region of γ-secretase cleavage; 5=linker; 6=TAT exon I; 7=linker; 8=amino acids 664-695 of APP.

FIG. 4A-B shows the DNA sequence (SEQ ID NO:3) of the fusion protein APP(1-651)wt, K612V-TATexonI(M1L) APP (664-695).

FIG. 5 shows the amino acid sequence (SEQ ID NO:4) of the fusion protein APP(1-651)wt, K612V-TATexonI(M1L) APP (664-695) with the various different regions of the fusion protein demarcated. 1=amino acids 1-651 of APP; 2=region of β-secretase cleavage; 3=K612V mutation; 4=region of γ-secretase cleavage; 5=linker; 6=TAT exon I; 7=linker; 8=amino acids 664-695 of APP.

FIG. 6A-C shows the DNA sequence (SEQ ID NO:5) of the fusion protein APP(1-651)SW, K612V, GAL4-VP16(delMet) APP (664-695).

FIG. 7 shows the amino acid sequence (SEQ ID NO:6) of the fusion protein APP(1-651)SW, K612V, GAL4-VP16(delMet) APP (664-695) with the various different regions of the fusion protein demarcated. 1=amino acids 1-651 of APP; 2=region of β-secretase cleavage; 3=K612V mutation; 4=region of γ-secretase cleavage; 5=linker; 6=GAL4-VP16; 7=linker; 8=amino acids 664-695 of APP.

FIG. 8A-C shows the DNA sequence (SEQ ID NO:7) of the fusion protein APP(1-651)wt, K612V, GAL4-VP16(del Met) APP (664-695).

FIG. 9 shows the amino acid sequence (SEQ ID NO:8) of the fusion protein APP(1-651)wt, K612V, GAL4-VP16(del Met) APP (664-695) with the various different regions of the fusion protein demarcated. 1=amino acids 1-651 of APP; 2=region of β-secretase cleavage; 3=K612V mutation; 4=region of γ-secretase cleavage; 5=linker; 6=GAL4-VP16; 7=linker; 8=amino acids 664-695 of APP.

FIG. 10A-B shows the DNA sequence (SEQ ID NO:9) of the fusion protein APP(1-651)SW, TATexonI(M1L) APP (664-695).

FIG. 11 shows the amino acid sequence (SEQ ID NO:10) of the fusion protein APP(1-651)SW, TATexonI(M1L) APP (664-695) with the various different regions of the fusion protein demarcated. 1=amino acids 1-651 of APP; 2=region of β-secretase cleavage; 3=wild-type K at position 612; 4=region of γ-secretase cleavage; 5=linker; 6=TAT exon I; 7=linker; 8=amino acids 664-695 of APP.

FIG. 12A-B shows the DNA sequence (SEQ ID NO:11) of the fusion protein APP(1-651)wt, TATexonI(M1L) APP (664-695).

FIG. 13 shows the amino acid sequence (SEQ ID NO:12) of the fusion protein APP(1-651)wt, TATexonI(M1L) APP (664-695) with the various different regions of the fusion protein demarcated. 1=amino acids 1-651 of APP; 2=region of 13-secretase cleavage; 3=wild-type K at position 612; 4=region of γ-secretase cleavage; 5=linker; 6=TAT exon I; 7=linker; 8=amino acids 664-695 of APP.

FIG. 14A-C shows the DNA sequence (SEQ ID NO:13) of the fusion protein APP(1-651)SW, GAL4-VP16(delMet) APP (664-695).

FIG. 15 shows the amino acid sequence (SEQ ID NO:14) of the fusion protein APP(1-651)SW, GAL4-VP16(delMet) APP (664-695) with the various different regions of the fusion protein demarcated. 1=amino acids 1-651 of APP; 2=region of β-secretase cleavage; 3=wild-type K at position 612; 4=region of γ-secretase cleavage; 5=linker; 6=GAL4-VP16; 7=linker; 8=amino acids 664-695 of APP.

FIG. 16A-C shows the DNA sequence (SEQ ID NO:15) of the fusion protein APP(1-651)wt, GAL4-VP16(delMet) APP (664-695).

FIG. 17 shows the amino acid sequence (SEQ ID NO:16) of the fusion protein APP(1-651)wt, GAL4-VP16(delMet) APP (664-695) with the various different regions of the fusion protein demarcated. 1=amino acids 1-651 of APP; 2=region of β-secretase cleavage; 3=wild-type K at position 612; 4=region of γ-secretase cleavage; 5=linker; 6=GAL4-4VP16; 7=linker; 8=amino acids 664-695 of APP.

FIG. 18A-B shows the cDNA sequence (SEQ ID NO:17) and FIG. 18C shows the amino acid sequence (SEQ ID NO:18) of the 695 amino acid splice variant of wild-type Alzheimer's precursor protein (APP). See GenBank accession no. Y00264 and Kang et al., 1987, Nature 325:733-736.

FIG. 19 shows data from an embodiment in which the assay of the present invention was used to identify both a β-secretase inhibitor and a γ-secretase inhibitor. See Example 3 for details.

FIG. 20 shows a schematic diagram of pCR2.1 Gal4-VP16.

FIG. 21A shows a schematic diagram of pRBR121. FIG. 21B shows a schematic diagram of pcDNA3.1 zeo (+) APP(1-651)SW, K612V-TATexonI(M1L) APP (664-695).

FIG. 22A shows a schematic diagram of pRBR186. FIG. 22B shows a schematic diagram of the viral plasmid pNL4-3. FIG. 22C shows a schematic diagram of pcDNA3.1 zeo (+) APP(1-651)SW, K612V-TATexonI(M1L) APP (664-695) with additional details as compared to FIG. 21B, which shows the same plasmid.

FIG. 23 shows a schematic diagram of pRSV Kan/Neo res.

FIG. 24 shows a schematic diagram of pUCd5TAT.

FIG. 25A shows a schematic diagram of pMM321. FIG. 25B-D shows the nucleotide sequence of pMM321. The upper strand is SEQ ID NO:19. The lower strand (SEQ ID NO:20) is the reverse complement of SEQ ID NO:19.

FIG. 26A shows a schematic diagram of the expression vector pcDNA3.1 zeo (+)APP(1-651)SW, K612V-(M1L)TATexonI. This expression vector directs the expression of a fusion protein containing the first 651 amino acids of APP with the Swedish version of the β-secretase cleavage site and the K612V mutation fused to the first exon of HIV1 TAT. The methionine at position 1 of TAT has been changed to leucine. FIG. 26B-G shows the nucleotide sequence of pcDNA3.1 zeo (+)APP(1-651)SW, K612V-(M1L)TATexonI. The upper strand is SEQ ID NO:21. The lower strand (SEQ ID NO:22) is the reverse complement of SEQ ID NO:21.

FIG. 27A-B shows a schematic diagram depicting general features of the present invention. FIG. 27A: The vertical bar represents a fusion protein with APP sequences represented as unfilled or lightly shaded portions of the bar. The lightly shaded portion represents Aβ. “BACE” indicates the β-secretase cleavage site. The dark shaded portion represents the transcription factor fused between APP sequences. The horizontal bar represents a membrane in which the uncleaved fusion protein is embedded, e.g., the endoplasmic reticulum. FIG. 27B: The transcription factor (plus small amounts of APP), having been released from the fusion protein and thus the membrane by APP processing, is shown in the nucleus binding to and activating the regulatory DNA sequence (“Transcription Factor Response Element”) that controls the expression of the reporter gene.

FIG. 28A-B shows the DNA sequence (SEQ ID NO:23) of the fusion protein APP(1-651)NFEV, K612V-TATexonI(M1L) APP (664-695).

FIG. 29 shows the amino acid sequence of a fusion protein (APP(1-651)NFEV, K612V-TATexonI(M1L) APP (664-695)) (SEQ ID NO:24) containing the sequence NFEV at the β-secretase cleavage site (underlined at 2). The other portions of the fusion protein are indicated as follows: 1=amino acids 1-651 of APP; 2=region of β-secretase cleavage; 3=K612V mutation; 4=region of γ-secretase cleavage; 5=linker; 6=TAT exon I; 7=linker; 8=amino acids 664-695 of APP.

FIG. 30A-C shows the DNA sequence (SEQ ID NO:25) of the fusion protein APP(1-651)NFEV, K612V, GAL4-VP16(delMet) APP (664-695).

FIG. 31 shows the amino acid sequence of a fusion protein (APP(1-651)NFEV, K612V, GAL4-VP16(delMet) APP (664-695)) (SEQ ID NO:26) containing the sequence NFEV at the β-secretase cleavage site (underlined at 2). The other portions of the fusion protein are indicated as follows: 1=amino acids 1-651 of APP; 2=region of β-secretase cleavage; 3=K612V mutation; 4=region of γ-secretase cleavage; 5=linker; 6=GAL4-VP16; 7=linker; 8=amino acids 664-695 of APP.

FIG. 32A shows a schematic diagram of pcDNA3.1 zeo (+), a eukaryotic expression vector that is suitable for use in the present invention. FIG. 32B-F shows the nucleotide sequence of pcDNA3.1 zeo (+). The upper strand is SEQ ID NO:27. The lower strand (SEQ ID NO:28) is the reverse complement of SEQ ID NO:27.

FIG. 33 shows data from an embodiment of the present invention utilizing a β-galactosidase reporter gene in which the assay of the present invention was used to identify both a β-secretase inhibitor and a γ-secretase inhibitor. See Example 8 for details.

FIG. 34 shows data from an embodiment of the present invention in which a fusion protein having a wild-type β-secretase cleavage site and a fusion protein having a Swedish β-secretase cleavage site are compared. See Example 9 for details.

FIG. 35A-B shows the DNA sequence (SEQ ID NO:29) of the fusion protein APP(1-651)NFEV, TATexonI(M1L) APP (664-695).

FIG. 36 shows the amino acid sequence of a fusion protein (APP(1-651)NFEV, TATexonI(M1L) APP (664-695)) (SEQ ID NO:30) containing the sequence NFEV at the β-secretase cleavage site (underlined at 2) and a wild-type K at position 612 (underlined at 3). The other portions of the fusion protein are indicated as follows: 1=amino acids 1-651 of APP; 2=region of β-secretase cleavage; 3=wild-type K; 4=region of γ-secretase cleavage; 5=linker; 6=TAT exon I; 7=linker; 8=amino acids 664-695 of APP.

FIG. 37A-C shows the DNA sequence (SEQ ID NO:3 1) of the fusion protein APP(1-651)NFEV, GAL4-VP16(delMet) APP (664-695).

FIG. 38 shows the amino acid sequence of a fusion protein (APP(1-651)NFEV, GAL4-VP16(delMet) APP (664-695)) (SEQ ID NO:32) containing the sequence NFEV at the β-secretase cleavage site (underlined at 2) and a wild-type K at position 612 (underlined at 3). The other portions of the fusion protein are indicated as follows: 1=amino acids 1-651 of APP; 2=region of β-secretase cleavage; 3=wild-type K; 4=region of γ-secretase cleavage; 5=linker; 6=GAL4-VP16; 7=linker; 8=amino acids 664-695 of APP.

### DETAILED DESCRIPTION OF THE INVENTION

For the purposes of this invention:

A “fusion protein” is a protein that contains at least two polypeptide regions and, optionally, a linking peptide to operatively link the two polypeptides into one continuous polypeptide. The at least two polypeptide regions in a fusion protein are derived from different sources, and therefore a fusion protein comprises two polypeptide regions not normally joined together in nature.

A “linking sequence (or linker peptide)” contains one or more amino acid residues joined in peptide bonds. A linking sequence serves to join two polypeptide regions of differing origins in a fusion protein via a peptide bond between the linking sequence and each of the polypeptide regions.

Typically, a fusion protein is synthesized as a continuous polypeptide in a recombinant host cell which contains an expression vector comprising a nucleotide sequence encoding the fusion protein where the different regions of the fusion protein are fused in frame on either side of a linker peptide's coding sequence. The chimeric coding sequence (encoding the fusion protein) is operatively linked to expression control sequences (generally provided by the expression vector) that are functional in the recombinant host cell.

“Reporter gene,” as used in the present invention, does not mean a DNA sequence present on the chromosome of a cell, generally possessing introns, as is often meant by the word “gene” in the art. Rather “reporter gene” means any DNA sequence encoding a protein or polypeptide that can give rise to a signal that can be detected or measured. “Reporter gene” does not mean a portion of the amino acid sequence of APP. “Reporter gene” will usually mean a DNA sequence, generally a cDNA sequence (although in some cases a reporter gene may have introns) that encodes a protein or polypeptide that is commonly used in the art to provide a measurable phenotype that can be distinguished over background signals.

A “nuclear localization signal (NLS)” is a region of a polypeptide which targets the polypeptide to the nucleus of the cell. One such NLS is that from the SV40 large T antigen. See, e.g., U.S. Pat. No. 5,589,392; Kalderon et al., 1984, Cell 39:499-509. The minimum region of the SV40 large T antigen with NLS activity is Pro-Lys-Lys-Lys-Arg-Lys-Val (SEQ ID NO:22). See also U.S. Pat. No. 5,776,689.

“Substances” that are screened in the present invention can be any substances that are generally screened in the pharmaceutical industry during the drug development process. For example, substances may be low molecular weight organic compounds (e.g., having a molecular weight of less than about 2,000 daltons and preferably less than about 1,000 daltons), RNA, DNA, antibodies, peptides, or proteins. Substances are often tested in the methods of the present invention as large collections of substances, e.g. libraries of low molecular weight organic compounds, peptides, or natural products.

The conditions under which substances are employed in the methods described herein are conditions that are typically used in the art for the study of protein-ligand interactions or enzyme inhibition studies: e.g., salt conditions such as those represented by such commonly used buffers as PBS or in tissue culture media; a temperature of about 4° C. to about 55° C.; incubation times of from several seconds to several hours or even up to 24 or 48 hours. Screening for the identification of enzyme-specific inhibitors is a well-known procedure in the pharmaceutical arts and the numerous conditions under which such screening has been done are available in the literature to guide the practitioner of he present invention.

A “conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutamic acid for aspartic acid); substitution of one aromatic amino acid (tryptophan, tyrosine, or phenylalanine) for another.

“Transfection” refers to any of the methods known in the art for introducing DNA into a cell, e.g., calcium phosphate or calcium chloride mediated transfection, electroporation, infection with a retroviral vector.

The present invention relates to the discovery of an assay system that permits the simultaneous screening for inhibitors of several types of amyloid precursor protein (APP) processing or signaling (e.g., β-secretase cleavage, γ-secretase cleavage, APP extracellular signaling, APP cytoplasmic signaling). In a preferred embodiment, this screening is accomplished without the concomitant identification of inhibitors of α-secretase. The assay system is carried out in a single type of cell, using a single type of assay readout. Inhibitors discovered by means of the present invention are expected to be useful in the treatment of Alzheimer's disease since these inhibitors are likely to be capable of interfering with the production of Aβ.

Previous assays for identifying inhibitors of APP processing have focussed specifically on inhibition of either β-secretase or γ-secretase activity, or on inhibition of some other single aspect of Aβ production. In contrast, the assays described herein are directed to inhibition of APP processing in general. Substances identified through these assays may target β-secretase, γ-secretase, modulators of β-secretase or γ-secretase activity, or even an as-yet-undiscovered ligand interaction with APP. In certain embodiments, these assays will also be free of the potentially misleading or obscuring effects of α-secretase activity. In addition, unlike other assays currently in use, these assays are homogeneous assays; i.e., they require no cumbersome or time-consuming steps such as column chromatography separations, immunoprecipitations, washing steps, etc. Therefore, the assays are very well adapted to a high throughput screening format.

In the present invention, novel recombinant DNA molecules are constructed in which nucleotide sequences encoding at least a portion of the luminal (i.e., N-terminal to the transmembrane region) and transmembrane regions of APP are fused to nucleotide sequences encoding a transcription factor. In a preferred embodiment, the APP contains an α-secretase cleavage site that has been altered to reduce or eliminate α-secretase cleavage. This allows the assays of the present invention to avoid identifying inhibitors of α-secretase and permits the more efficient detection of β-secretase inhibitors since α-secretase and β-secretase compete for APP cleavage. The recombinant DNA molecules may be transfected, along with a reporter gene, into a cell line that processes APP into Aβ, and stable clones may be generated. Alternatively, the recombinant DNA molecules and reporter plasmid may be utilized in transient transfections.

Upon expression in cells, the APP/transcription factor fusion protein localizes to a non-nuclear membrane of the cell (e.g., the endoplasmic reticulum) due to the presence of the APP sequences in the fusion protein. In a manner similar to cleavage of APP, the fusion protein will then be cleaved, first by β-secretase and then by γ-secretase. γ-secretase cleavage releases the transcription factor from the membrane in which the APP/transcription factor fusion protein had been embedded, after which the transcription factor translocates to the nucleus and stimulates transcription of the reporter gene. Assuming no α-secretase cleavage, cleavage by both β-secretase and γ-secretase is required for release of the transcription factor and transactivation of the reporter gene in this assay since γ-secretase cleavage of APP is dependent on a short luminal domain, such as that generated by β- or α-secretase cleavage. Detection of a signal from the reporter gene product will thus serve as evidence of APP processing. In particular, since activation of the reporter gene requires both β-secretase and γ-secretase cleavage, the assay is capable of identifying inhibitors of both or either of these proteases.

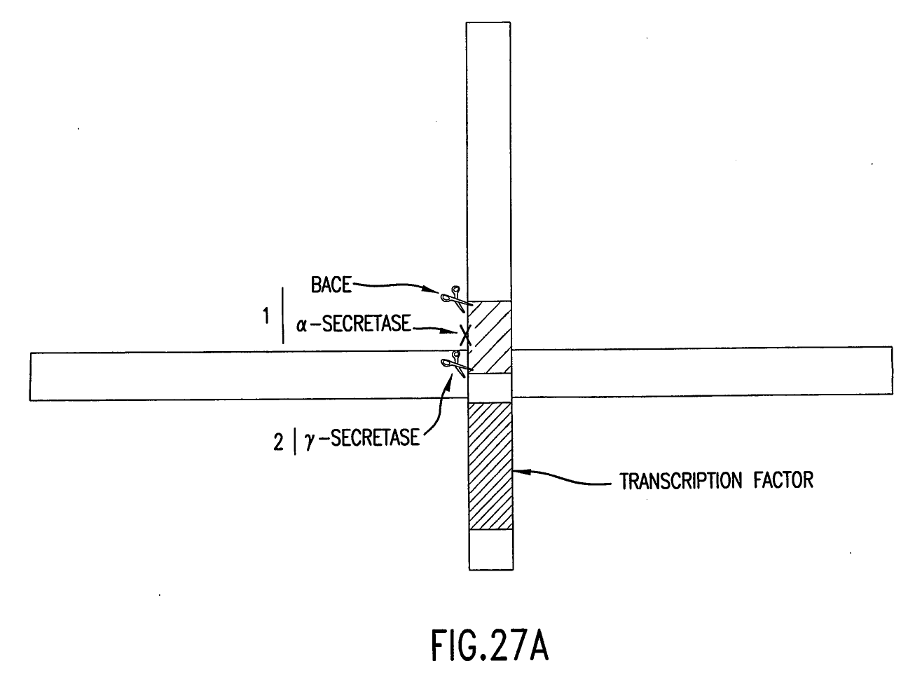

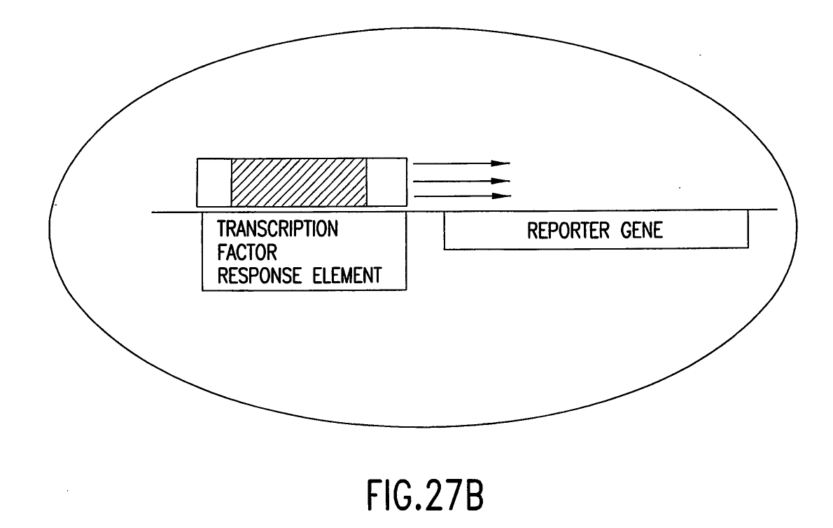

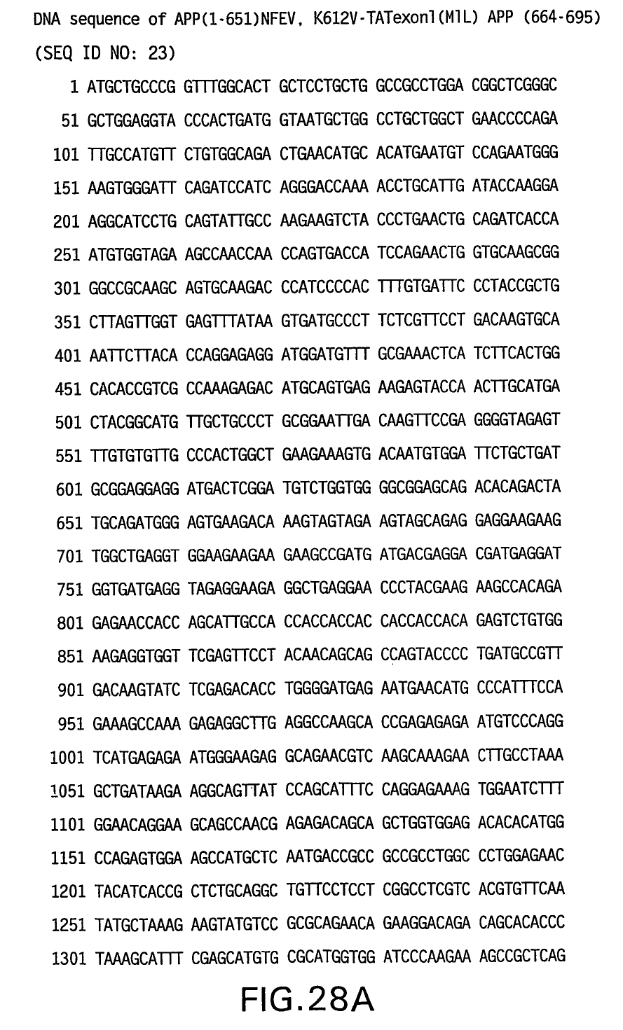

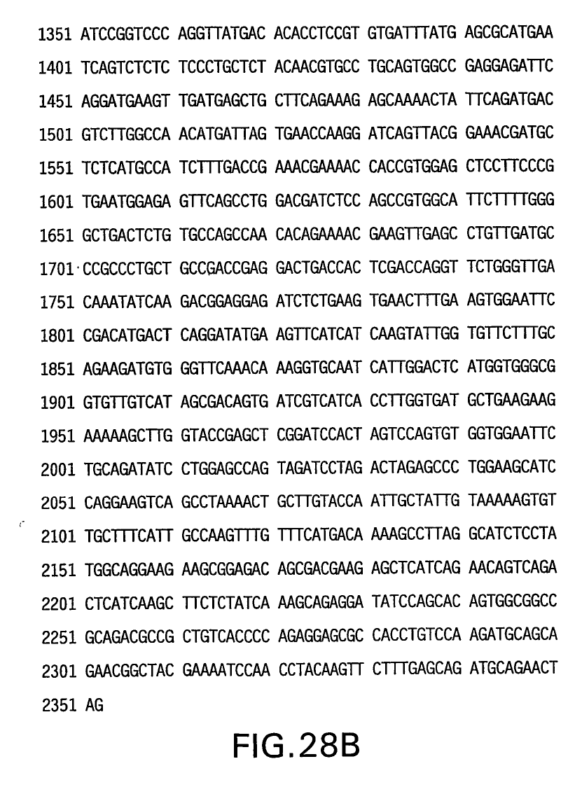

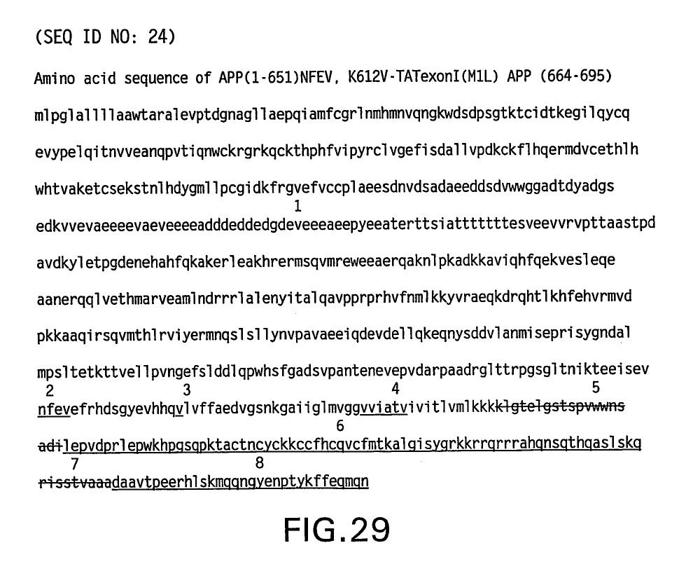

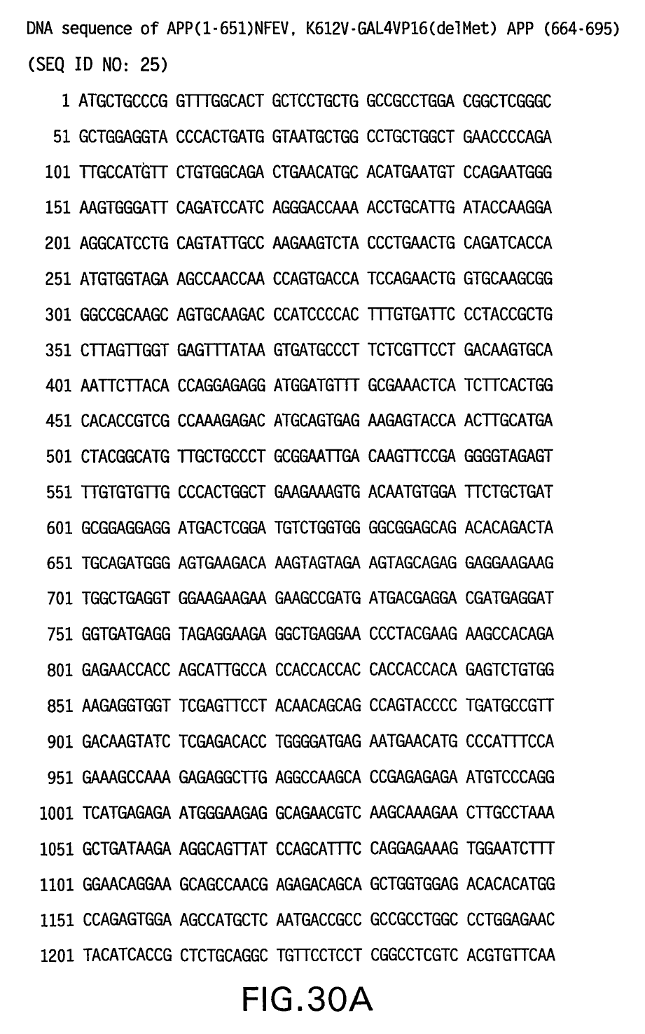

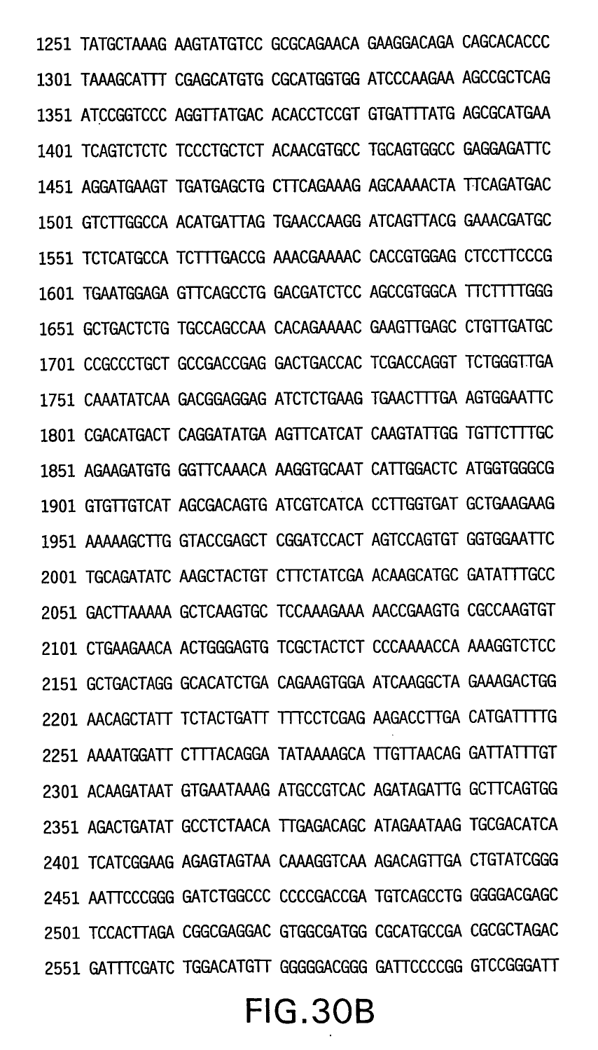

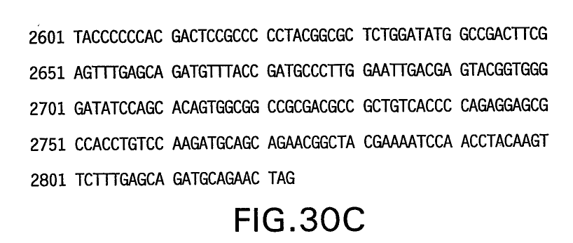

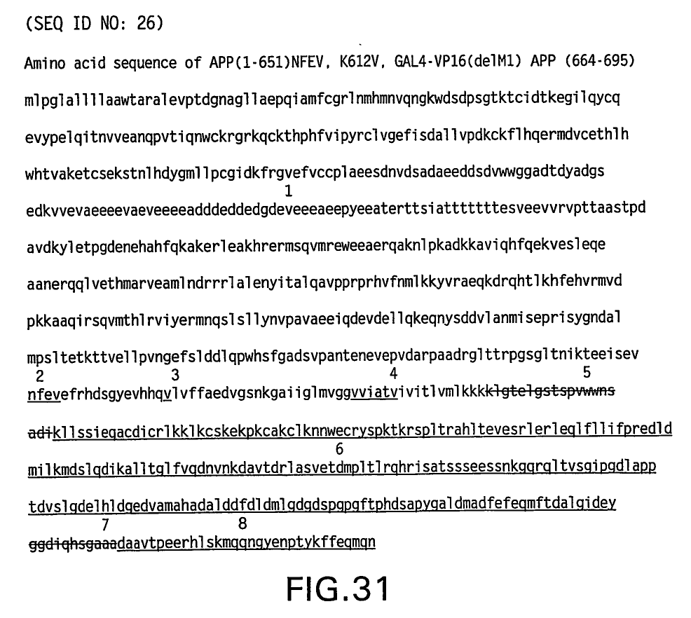

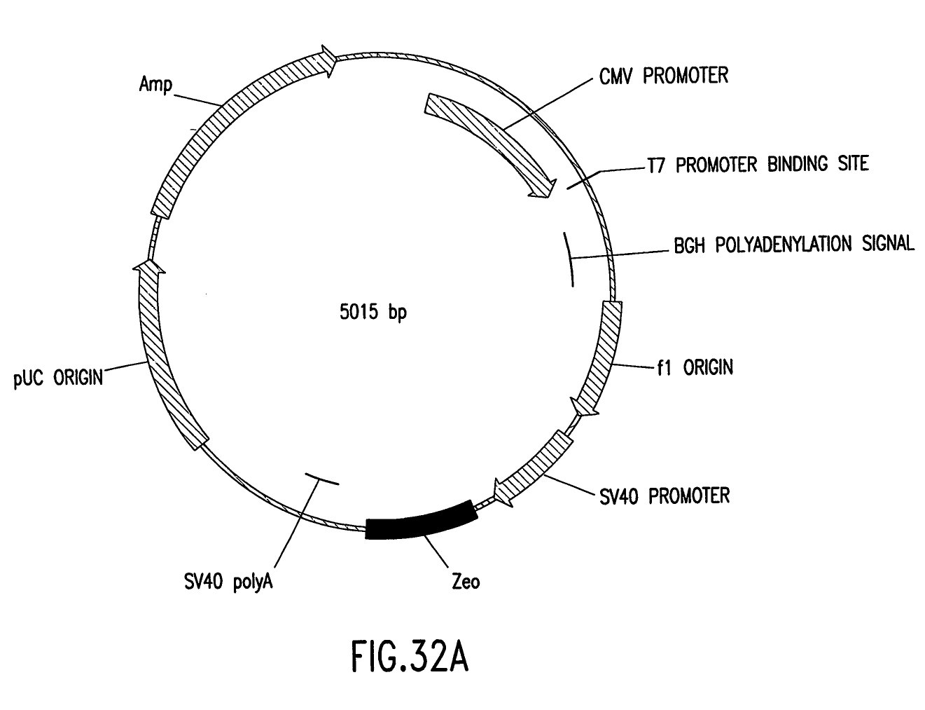

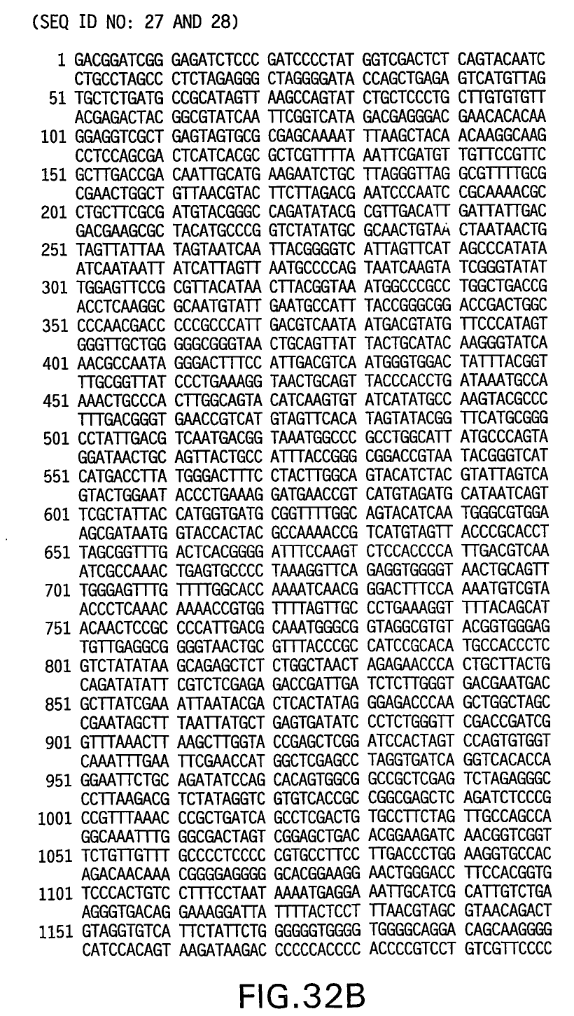

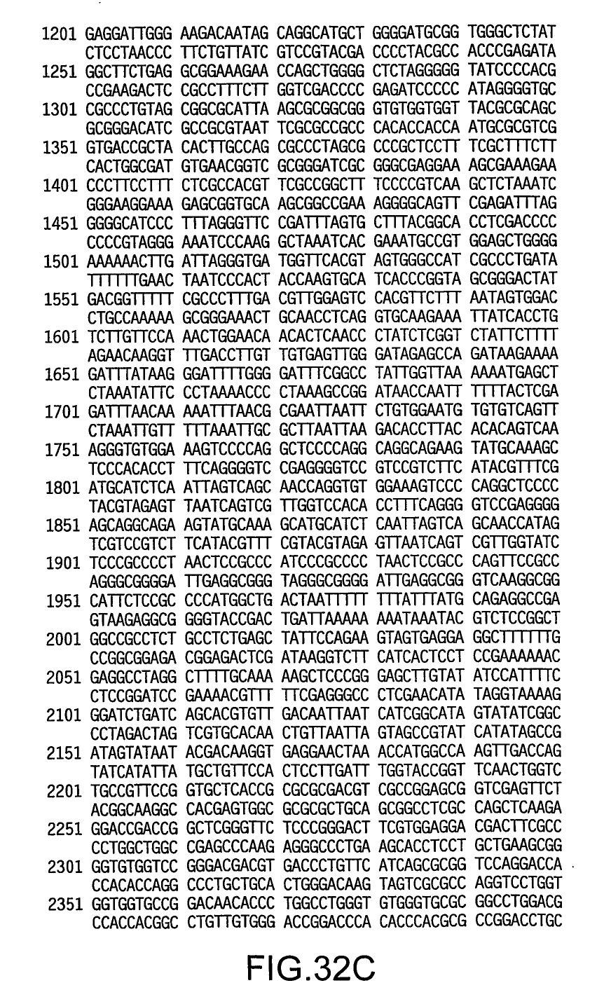

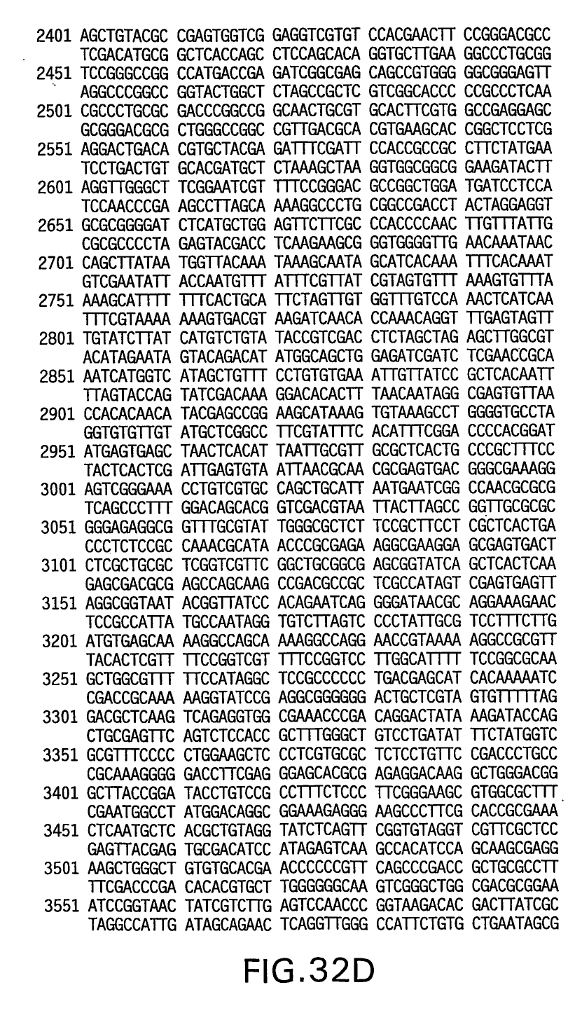

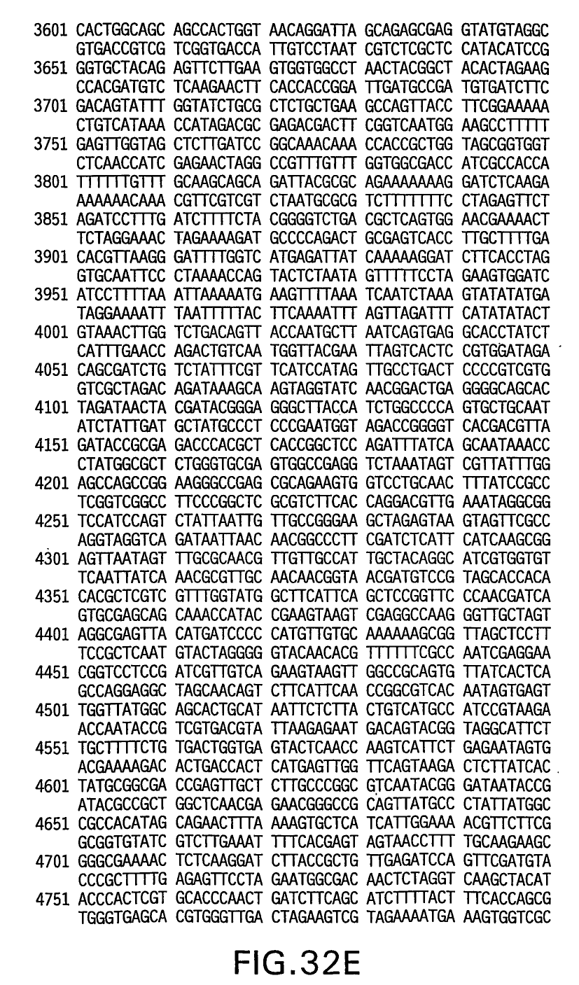

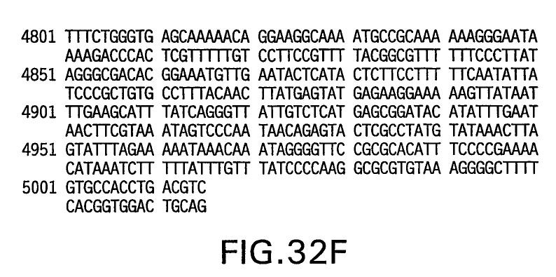

FIG. 27 is a schematic diagram depicting general features of the assay. The vertical bar in FIG. 27A represents the fusion protein; the horizontal bar represents the non-nuclear membrane in which the fusion protein is embedded before processing. FIG. 27B shows how the transcription factor portion of the fusion protein (with small amounts of the APP portion flanking it) has moved to the nucleus following release from the fusion protein by APP processing. In the nucleus, the transcription factor is shown binding to a regulatory DNA sequence (“Transcription Factor Response Element”) and activating transcription of the reporter gene.

The recombinant DNA molecules encoding the APP/transcription factor fusion protein and the reporter gene can be used to develop novel homogenous cell-based assays for the identification and assessment of inhibitors of APP processing which will be readily amenable to high throughput technology.

In one embodiment, the recombinant DNA molecules used in this invention comprise sequences encoding the amino terminal 651 amino acids of the 695 amino acid version of APP (Kang et al., 1987, Nature 325:733-736), including all the sequences necessary for the production of Aβ, as well as the C-terminal 32 amino acids of APP. The transcription factor is placed between the N-terminal and C-terminal portions of APP. The APP sequence may include a modification to increase the amount of β-secretase cleavage of the fusion protein. This modification involves mutating the K at position 612 of the α-secretase cleavage site to a V (K612V). Since α-secretase and β-secretase compete for APP cleavage, reducing or eliminating APP cleavage by α-secretase results in increased β-secretase cleavage, and allows the assay to detect β-secretase inhibitors more readily. In addition, the β-secretase cleavage site within APP (KM↓DA) (SEQ ID NO:34) may be modified, e.g., to that of a naturally occurring mutation (termed the “Swedish” mutation or NL↓DA) (SEQ ID NO:38) which has been shown to enhance β-secretase cleavage six-fold in cultured cells. Another possible modification is to replace the (KM↓DA) (SEQ ID NO:34) wild-type β-secretase cleavage site with the sequence (NF↓EV) (SEQ ID NO:40). The presence of NFEV in an amino acid sequence has been shown to enhance β-secretase cleavage by an even larger amount than the Swedish sequence. See U.S. Provisional Patent Application Ser. No. 60/292,591 and U.S. Provisional Patent Application Ser. No. 60/316,115, the disclosures of which are incorporated herein, in their entirety.

In a preferred embodiment, HIV-1 TAT exon I has been fused between sequences encoding the first 651 amino acids of APP₆₉₅ and the last 32 amino acids of APP₆₉₅ (APP-TAT-APPct32). Co-transfection of an expression vector comprising this construct with a reporter gene plasmid containing an HIV-1 LTR promoter that controls the transcription of a reporter gene leads to enhanced expression of the reporter gene. Other transcription factors that could be fused to APP₁₋₆₅₁ include Gal4-VP16, the entire Gal4 protein, BIV TAT, HIV-2 TAT, SIV TAT, LexA-VP16, EBV Zta, Papillomavimus E2, or tissue or species specific homodimeric bHLH transcription factors capable of activating transcription through specific DNA response elements, such as E12, E47, or Twist. The use of GAL4, BIV, HIV-2, or SIV TAT may be useful if it is desired to reduce the potency of the transactivator, thus reducing any background transactivation caused by non-specific cleavage of the fusion protein. To further reduce the potential for transactivation by TAT in the absence of β-secretase and γ-secretase cleavage, the TAT portion of the fusion protein may be altered to remove the N-terminal methionine and thus eliminate the possibility of aberrant translation of TAT through any potential internal ribosomal entry sites.

In some circumstances, high level expression of TAT has been found to be toxic to cells. Thus, when TAT is the transcription factor fused to APP in the methods of the present invention, it may be advantageous to utilize transient transfection with low amounts of the expression vector encoding the APP/TAT fusion protein. A set of preliminary experiments in which various amounts of the vector are transfected, in order to titrate acceptable levels of TAT, is recommended.

The reporter gene used will depend in large part upon the transcription factor fused to APP. The promoter used to drive the reporter gene will be LTR for TAT-based APP fusion proteins, or UAS (6×) for GAL4-VP16-based APP fusion proteins. In a particular embodiment, an LTR driving EGFP (enhanced green fluorescent protein, a brighter variant of GFP made by Aurora Biosciences, San Diego, Calif.) has been used to observe processing of an APP/TAT fusion protein. Under certain conditions, it may be desirable to use a less stable reporter, such as dsEGFP (a destabilized variant of EGFP made by Aurora Biosciences, San Diego, Calif. and marketed by Clontech, Palo Alto, Calif.) or a more potent reporter, such as β-lactamase. Alternatively, a stable HeLa cell line expressing LTR-β-galactosidase can be used. If the exquisite sensitivity of β-lactamase makes it less than optimal for a particular purpose, the LTR-β-galactosidase cell line may be exploited for this assay. Finally, under some circumstances Gal4-VP16 may prove to be optimal relative to TAT to reduce any inherent background problems associated with using the weakly but constitutively active LTR in the reporter plasmid, in which case the reporter plasmid could be UAS(6×)-β-lactamase (Aurora Biosciences, San Diego, Calif.).

A variety of cells are suitable for use in the methods of the present invention. Particularly preferred are eukaryotic, especially mammalian, cell lines. In particular embodiments, the cells are selected from the group consisting of: L cells L-M(TK⁻) (ATCC CCL 1.3), L cells L-M (ATCC CCL 1.2), HEK293 (ATCC CRL 1573), HEK293T, Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), T24 (ATCC HTB-4), PC12 cells, Jurkat cells, H4 cells (ATCC HTB-148), and MRC-5 (ATCC CCL 171).

To make the assay more amenable for ultra-high throughput screening, a non-adherent cell line, such as Jurkat, can be used.

Generally, the assays of the present invention employ cells that naturally express β-secretase and γ-secretase. However, it is possible to practice the invention in cells that lack the expression of one, or both, of these enzymes. In such cases, β-secretase and γ-secretase activity can be provided by the recombinant expression of these enzymes in the cells.

In one embodiment, the present invention provides a recombinant cell, preferably a eukaryotic cell, even more preferably a mammalian cell, and most preferably a human cell, where the cell expresses a fusion protein of APP and a transcription factor and the cell contains a reporter gene that can be activated by the transcription factor. The fusion protein comprises a portion of APP where that portion includes the regions of the β-secretase and γ-secretase cleavage sites fused to a transcription factor. The region of APP including the β-secretase and γ-secretase cleavage sites can be, e.g., a portion of APP that includes amino acids 589-651 of the 695 amino acid version of APP. This region is shown below.

| | |
|---|---|
| ↓ | |
| EEISEVKM DAEFRHDSGYEVHHQKLVFFAEDVGS | (SEQ ID NO:33) |
| ↓ ↓ | |
| NKGAIIGLMVGGVVIA TVIVITLVMLKKK | |

The β-secretase cleavage site is shown at position 596-597

| | |
|---|---|
| ↓ | |
| (KM DA). | (SEQ ID NO:34) |

Two predominant cleavage sites of γ-secretase are shown at positions 636-637 and 638-639

| | |
|---|---|
| ↓ ↓ | |
| (GVV IA TV). | (SEQ ID NO > 35) |

The fusion protein will be anchored in the membrane by the APP sequences shown above. The N-terminal portion of APP must include at least the β-secretase cleavage site, and possibly several amino-acids N-terminal to the β-secretase cleavage site to make the assay sensitive to both β-secretase and γ-secretase inhibitors. In many cases, the APP sequences will include sequences further N-terminal than those shown above, including the signal sequence at the N-terminus of APP. In cases, where the APP signal sequence is not used, another signal sequence may be incorporated in the fusion protein. Such other signal sequences are known in the art.

In a related embodiment, the present invention provides a recombinant cell, preferably a eukaryotic cell, even more preferably a mammalian cell, and most preferably a human cell, where the above-described APP portion of the fusion protein contains a K612V mutation. The APP portion of this embodiment is shown below.

| | |
|---|---|
| ↓ | |
| EEISEVKM DAEFRHDSGYEVHHQVLVFFAEDVGS | (SEQ ID NO:36) |
| ↓ ↓ | |
| NKGAIIGLMVGGVVIA TVIVITLVMLKKK | |

The β-secretase cleavage site is shown at position 596-597

| | |
|---|---|
| ↓ | |
| (KM DA). | (SEQ ID NO:34) |

Two predominant cleavage sites of γ-secretase are shown at positions 636-637 and 638-639

| | |
|---|---|
| ↓ ↓ | |
| (GVV IA TV). | (SEQ ID NO:35) |

The underlined V at position 612 shows the change in sequence in the present invention from the wild-type K to the mutant V, which change provides for reduced cleavage by α-secretase.

In a related embodiment, the present invention provides a recombinant cell, preferably a eukaryotic cell, even more preferably a mammalian cell, and most preferably a human cell, where the above-described APP portion of the fusion protein contains the Swedish version of the β-secretase cleavage site as well as a K612V mutation. The APP portion of this embodiment is shown below.

| | |
|---|---|
| ↓ | |
| EEISEVNL DAEFRHDSGYEVHHQVLVFFAEDVGS | (SEQ ID NO:37) |
| ↓ ↓ | |
| NKGAIIGLMVGGVVIA TVIVITLVMLKKK | |

The β-secretase cleavage site is shown at position 596-597

| | |
|---|---|
| ↓ | |
| (NL DA). | (SEQ ID NO:38) |

Two predominant cleavage sites of γ-secretase are shown at positions 636-637 and 638-639

| | |
|---|---|
| ↓ ↓ | |
| (GVV IA TV). | (SEQ ID NO:35) |

The underlined V at position 612 shows the change in sequence in the present invention from the wild-type K to the mutant V, which change provides for reduced cleavage by α-secretase.

In a related embodiment, the present invention provides a recombinant cell, preferably a eukaryotic cell, even more preferably a mammalian cell, and most preferably a human cell, where the above-described APP portion of the fusion protein contains the NFEV version of the β-secretase cleavage site as well as a K612V mutation. The APP portion of this embodiment is shown below.

| | |
|---|---|
| ↓ | |
| EEISEVNF EVEFRHDSGYEVHHQVLVFFAEDVGS | (SEQ ID NO:39) |
| ↓ ↓ | |
| NKGAIIGLMVGGVVIA TVIVITLVMLKKK | |

The β-secretase cleavage site is shown at position 596-597

| | |
|---|---|
| ↓ | |
| (NF EV). | (SEQ ID NO:40) |

Two predominant cleavage sites of γ-secretase are shown at positions 636-637 and 638-639

| | |
|---|---|
| ↓ ↓ | |
| (GVV IA TV). | (SEQ ID NO:35) |

The underlined V at position 612 shows the change in sequence in the present invention from the wild-type K to the mutant V, which change provides for reduced cleavage by α-secretase.

The presence of both β-secretase and γ-secretase cleavage sites in the fusion proteins permits the assays of the present invention to detect inhibitors of both β-secretase and γ-secretase.

The recombinant host cells of the present invention can be further engineered to comprise a reporter gene construct. The reporter gene construct contains a reporter gene in operable linkage with a regulatory DNA sequence that confers on the reporter gene the property of being regulated by the transcription factor of the fusion protein. This regulation is such that expression of the reporter gene is low or absent without binding of the transcription factor to the regulatory DNA sequence but, when the transcription factor is released from the fusion protein by APP processing, the transcription factor can move into the nucleus of the cell and bind to the regulatory DNA sequence, thereby activating transcription from the reporter gene.

Reporter genes desirably give rise to gene products which can be detected or quantitated, either in terms of amount of protein synthesized, enzymatic activity, fluorescence, luminescence, or some other phenotype. Suitable reporter gene products include firefly luciferase (de Wet et al., 1987, Mol. Cell. Biol. 7:725-737) or bacterial luciferase (Englebrecht et al., 1985, Science 227:1345-1347; Baldwin et al., 1984, Biochem. 23:3663-3667), β-lactamase, β-glucuronidase, β-galactosidase, green fluorescent proteins, enhanced green fluorescent protein, destabilized enhanced green fluorescent protein, red fluorescent protein, yellow fluorescent protein, cyan fluorescent protein, destabilized yellow fluorescent protein, destabilized cyan fluorescent protein, aequorin, chloramphenicol acetyl transferase (Alton & Vapnek, 1979, Nature 282:864-869), rat liver alkaline phosphatase (Toh et al., 1989, Eur. J. Biochem. 182:231-237), human placental secreted alkaline phosphatase (Cullen & Mallim, 1992, Meth. Enzymol. 216:362-368), and horseradish peroxidase, among others.

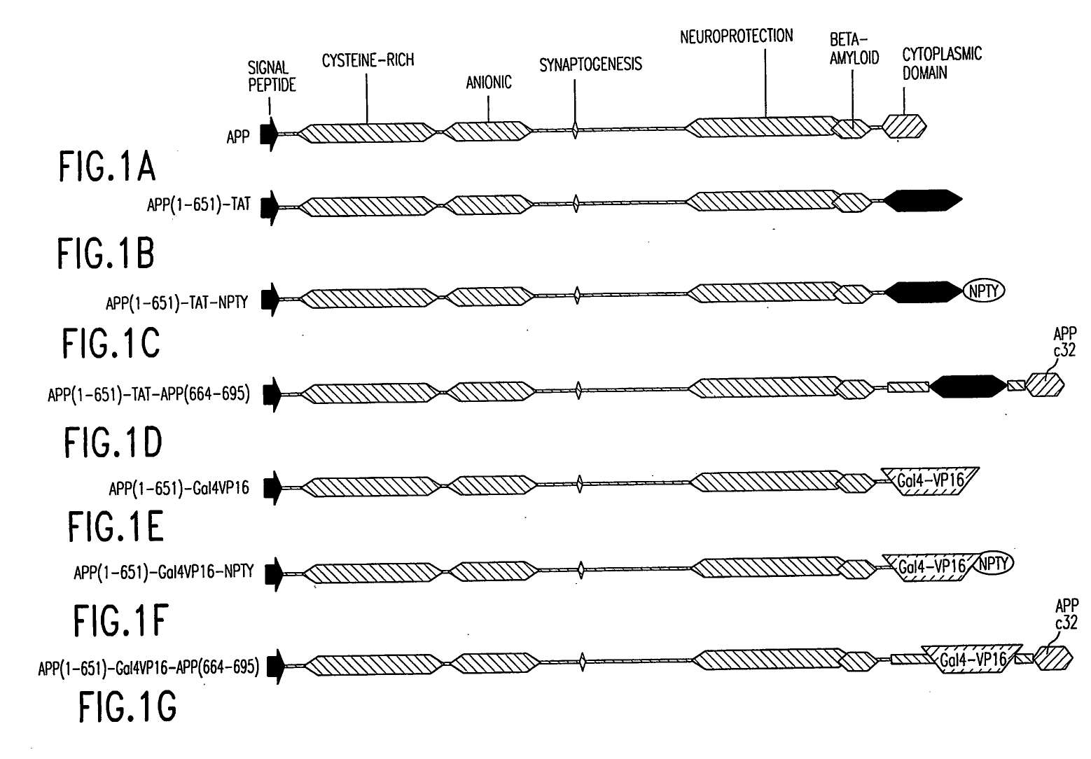

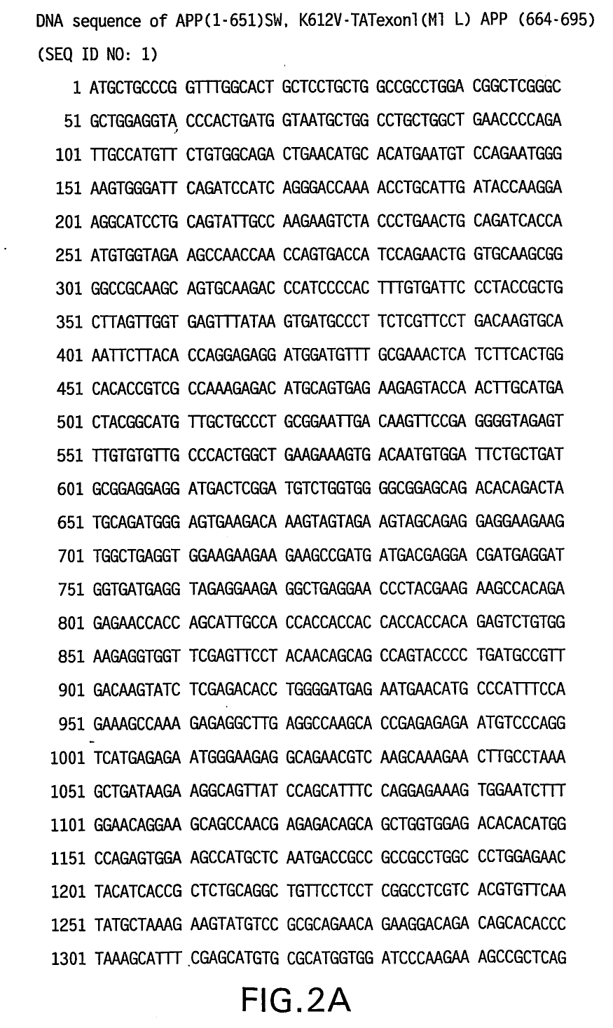

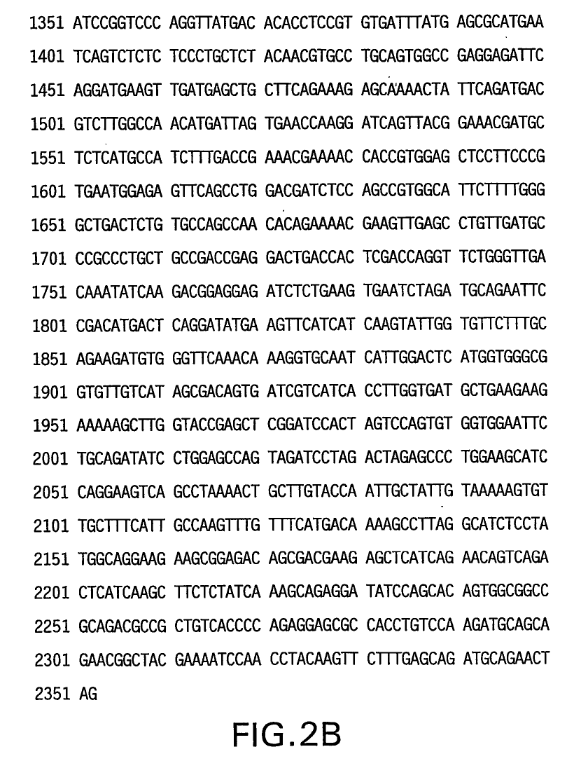

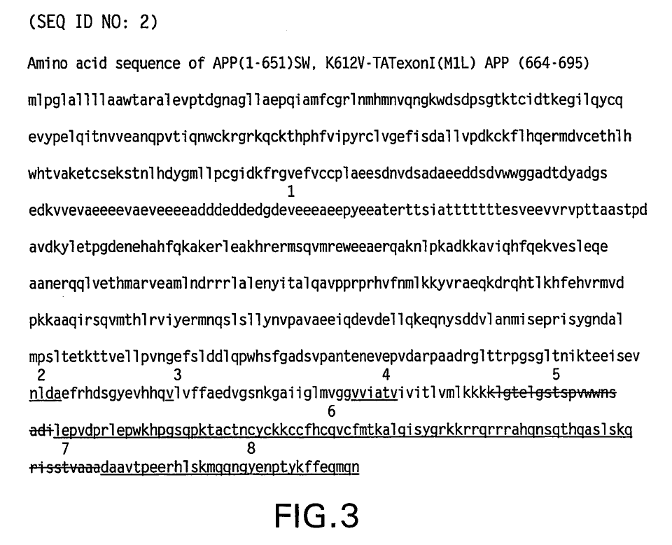

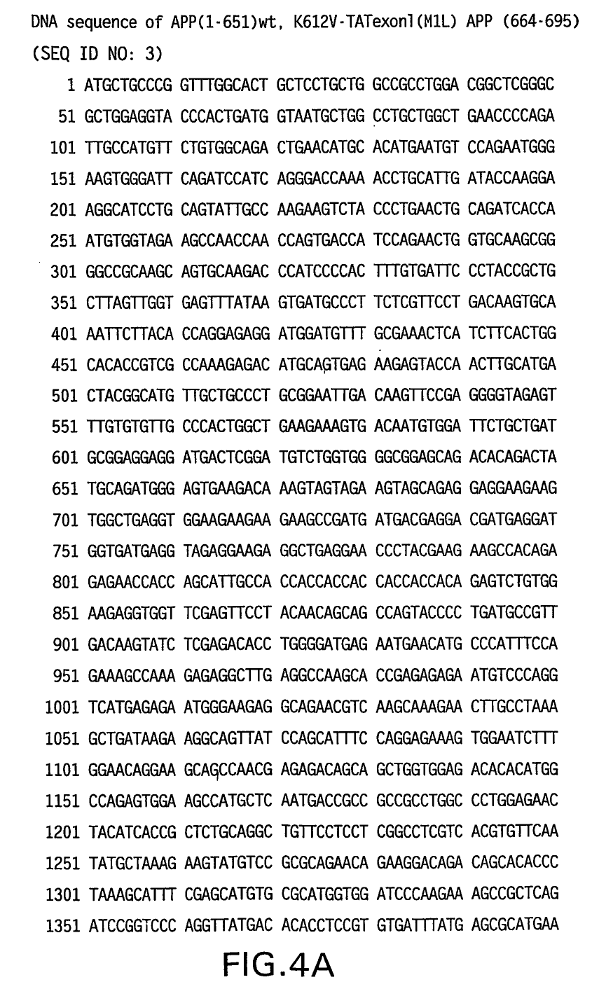

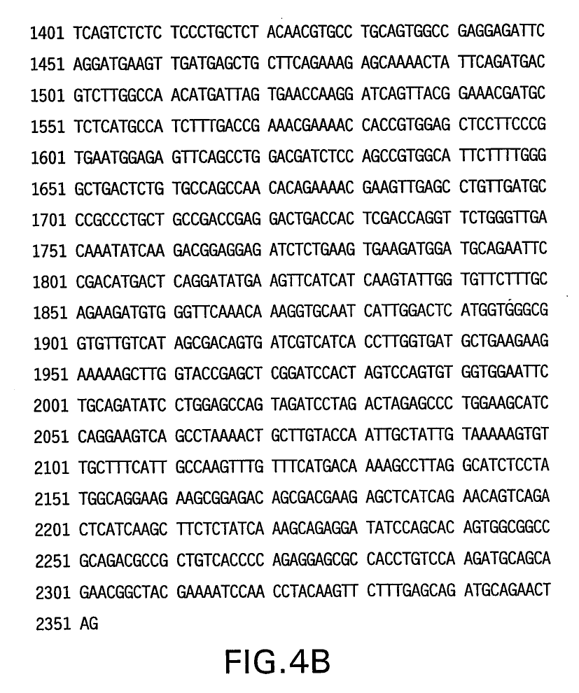

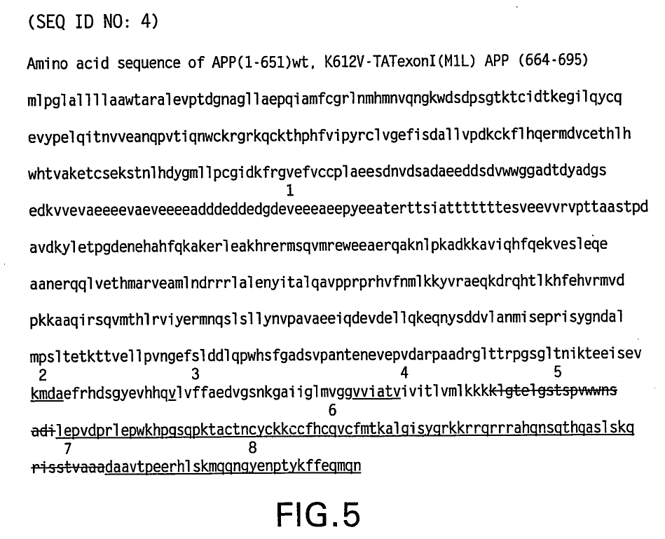

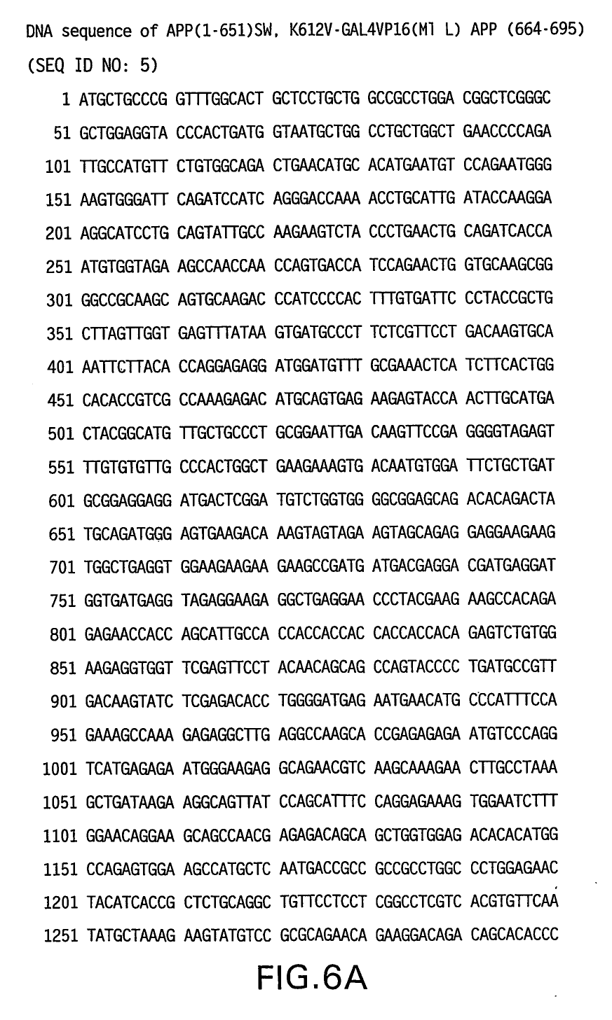

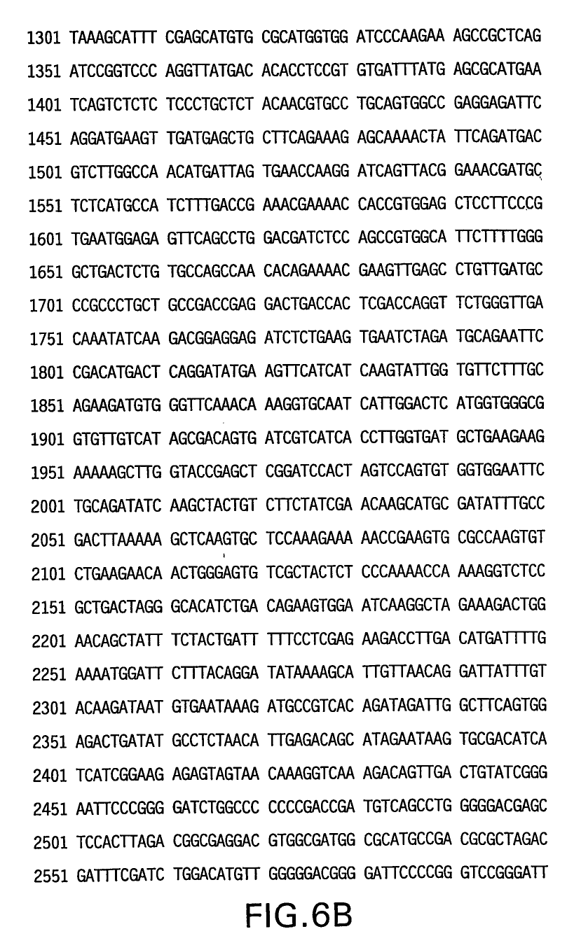

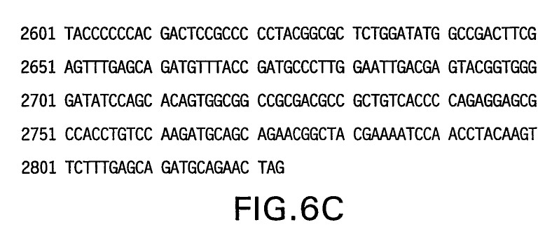

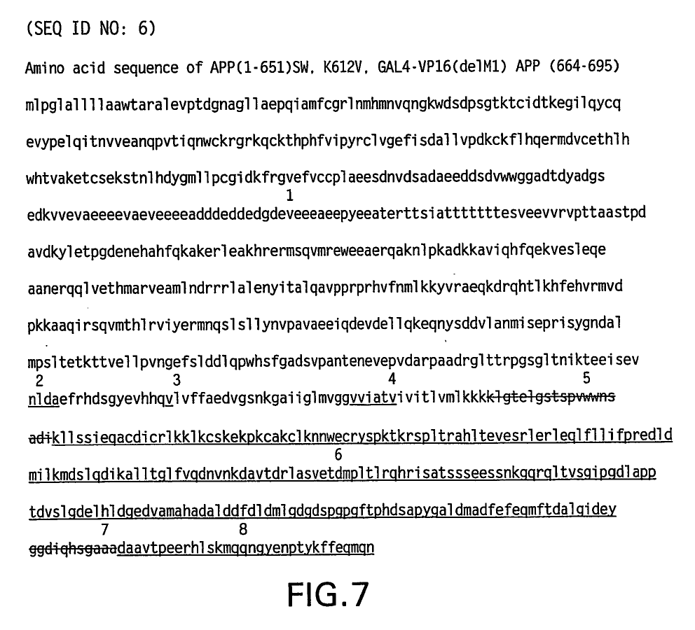

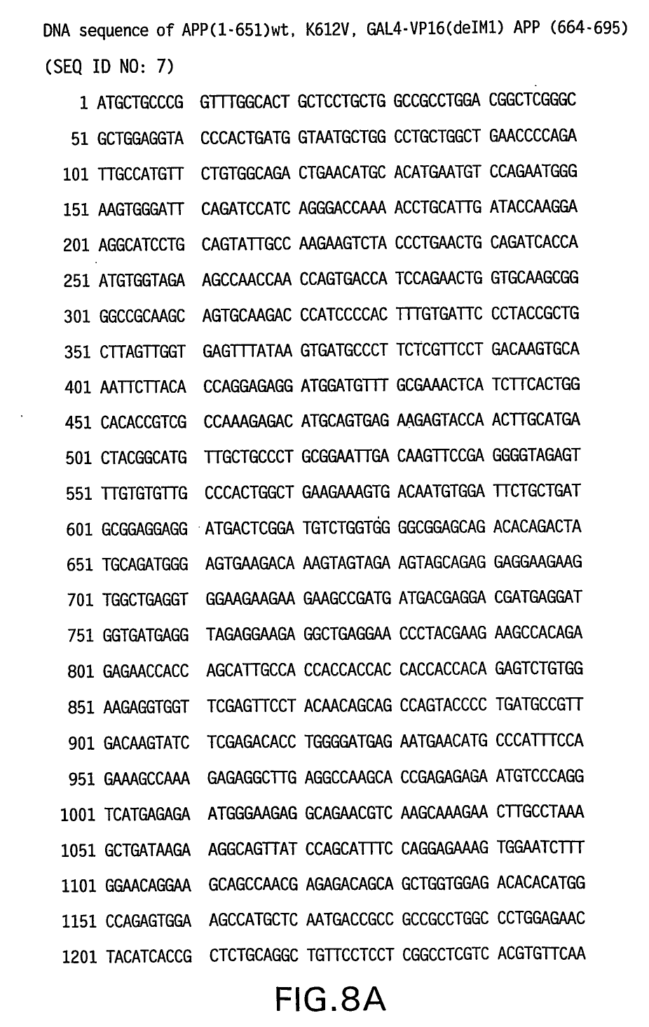

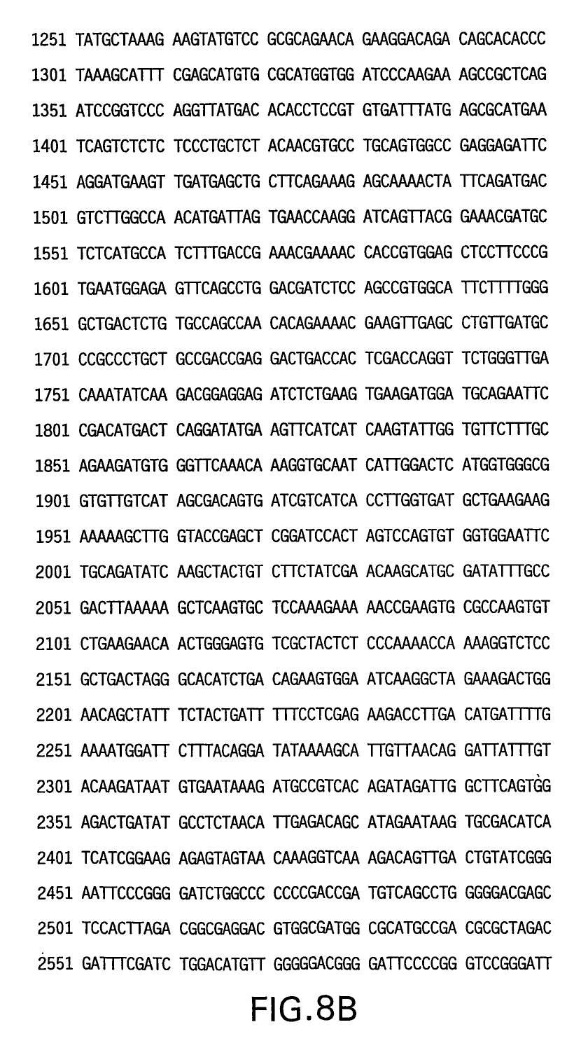

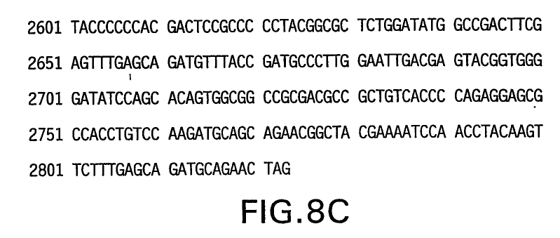

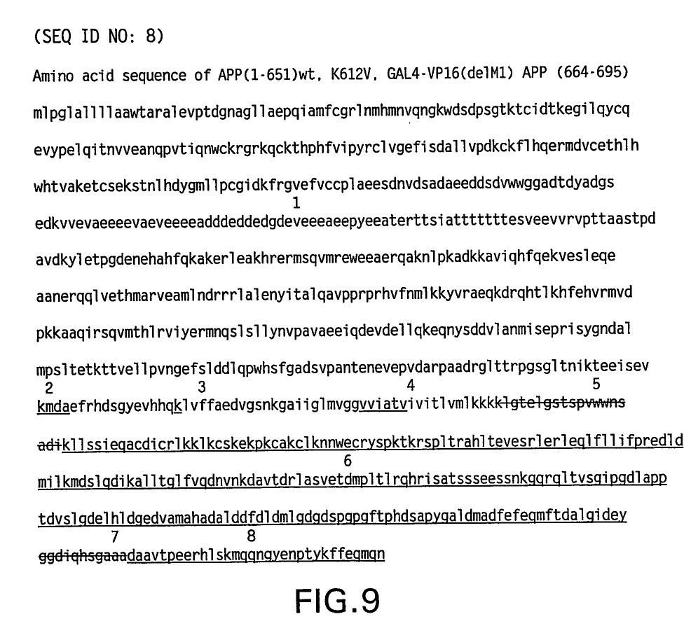

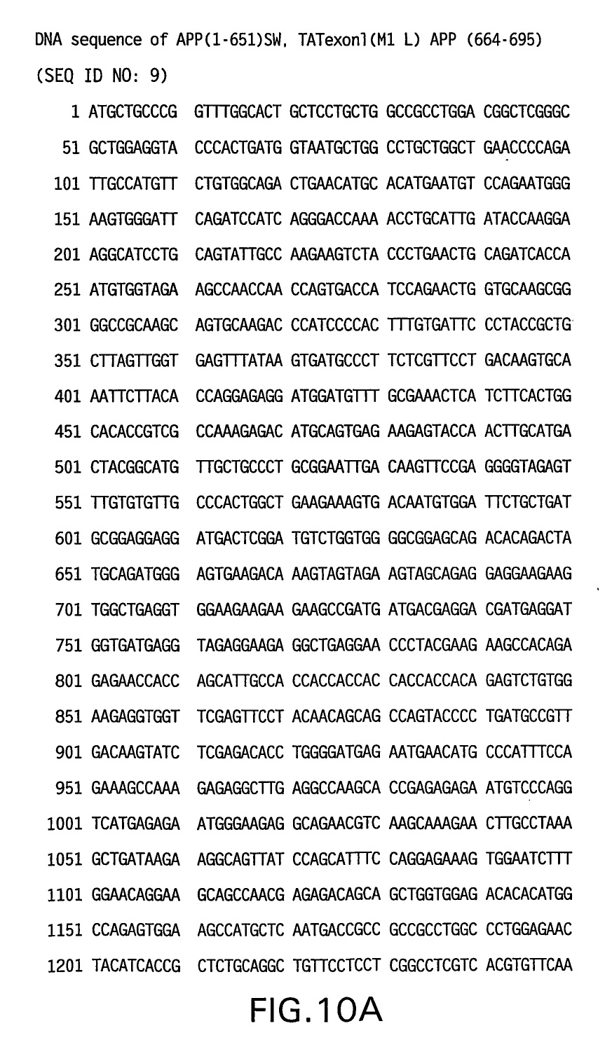

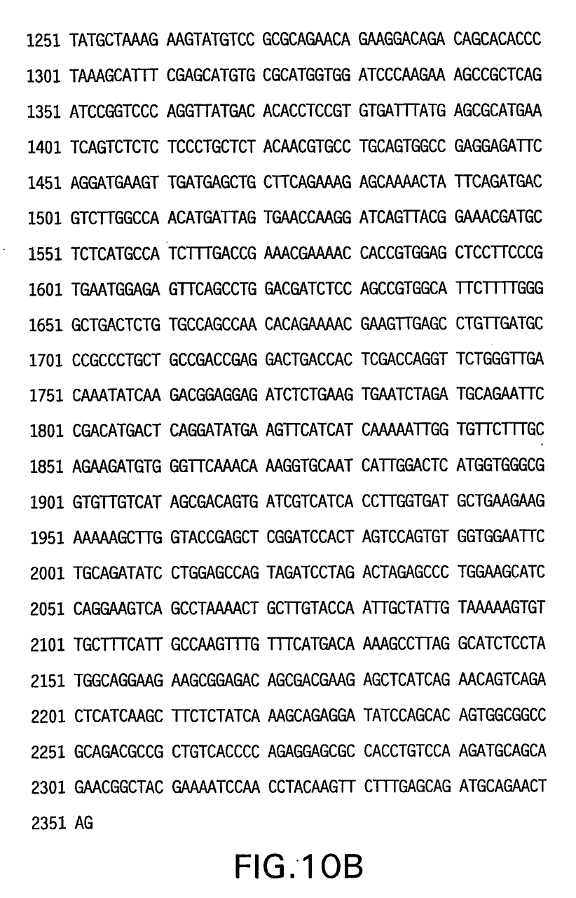

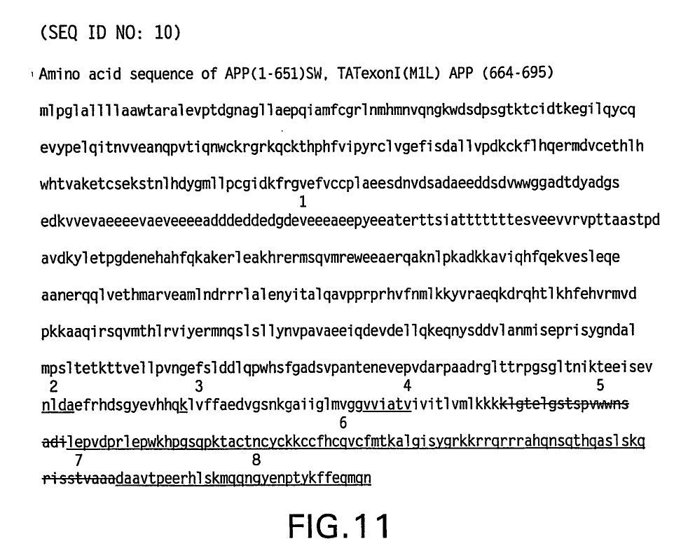

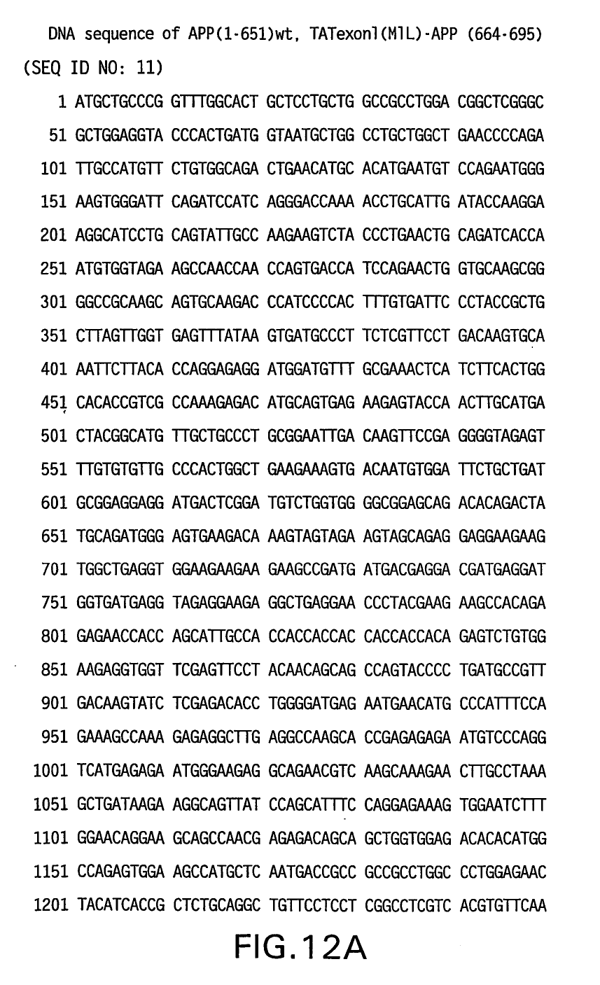

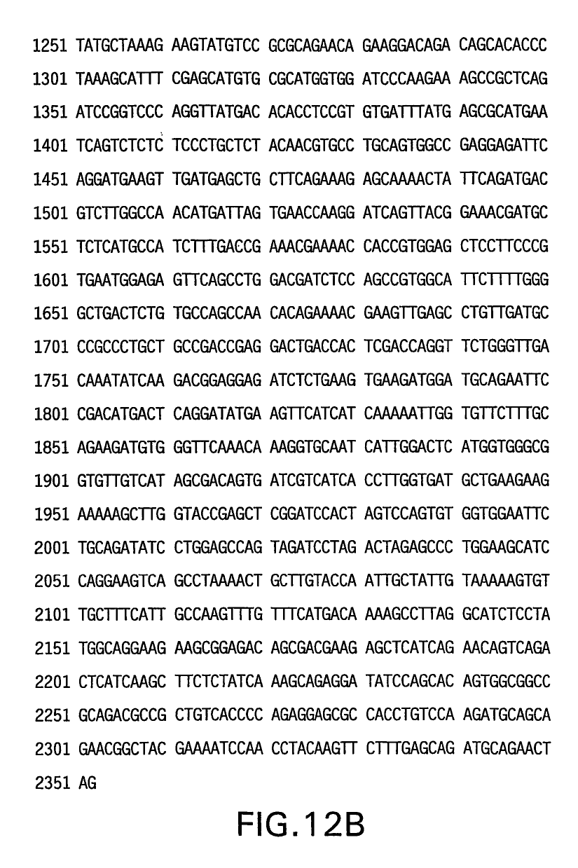

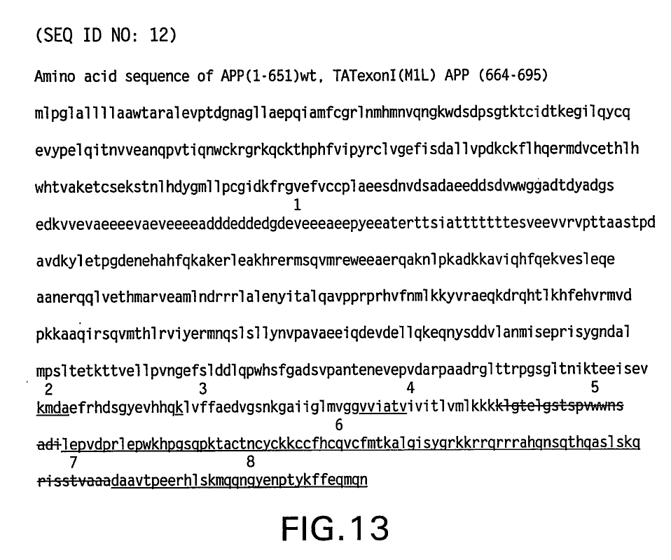

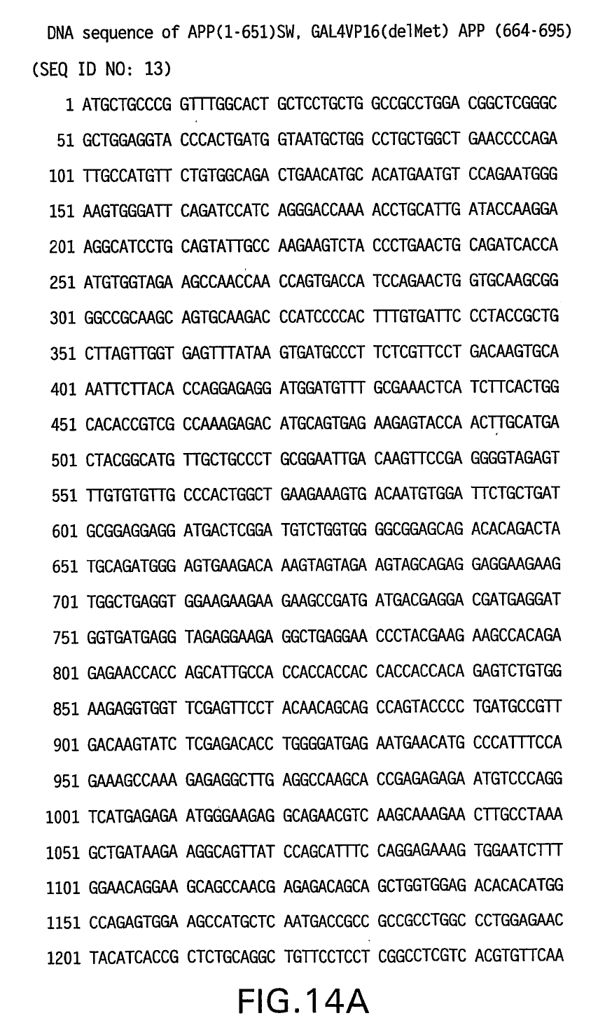

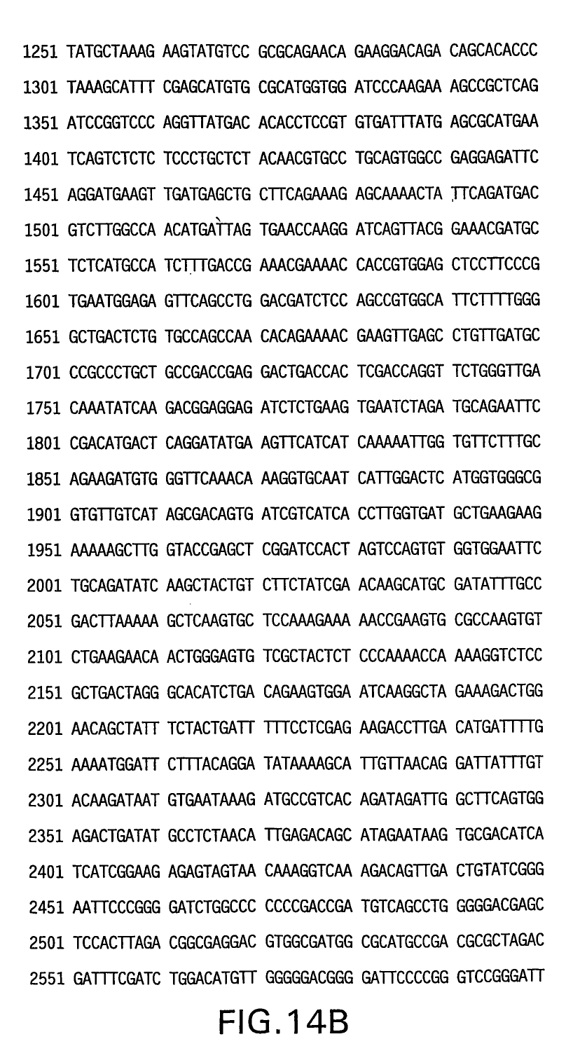

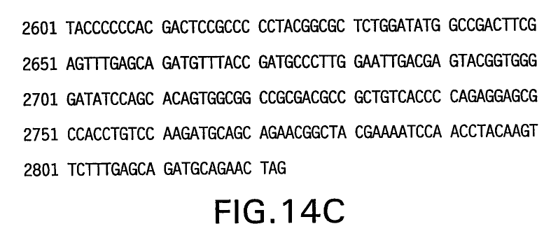

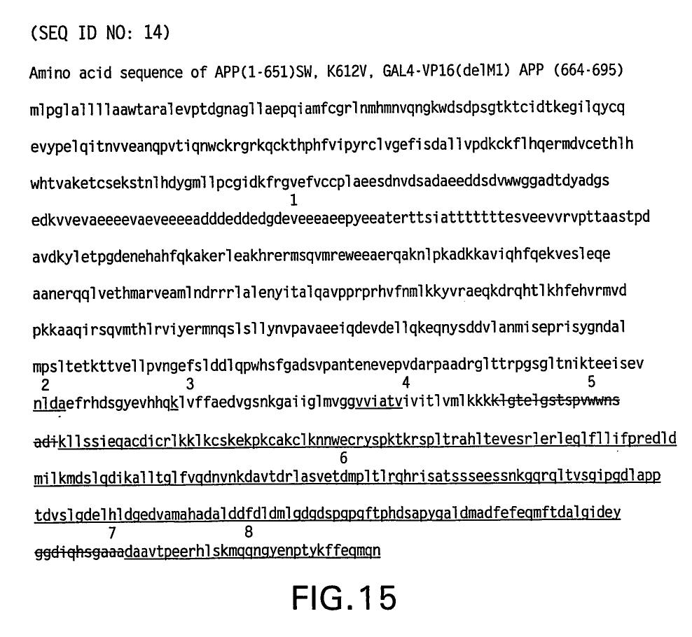

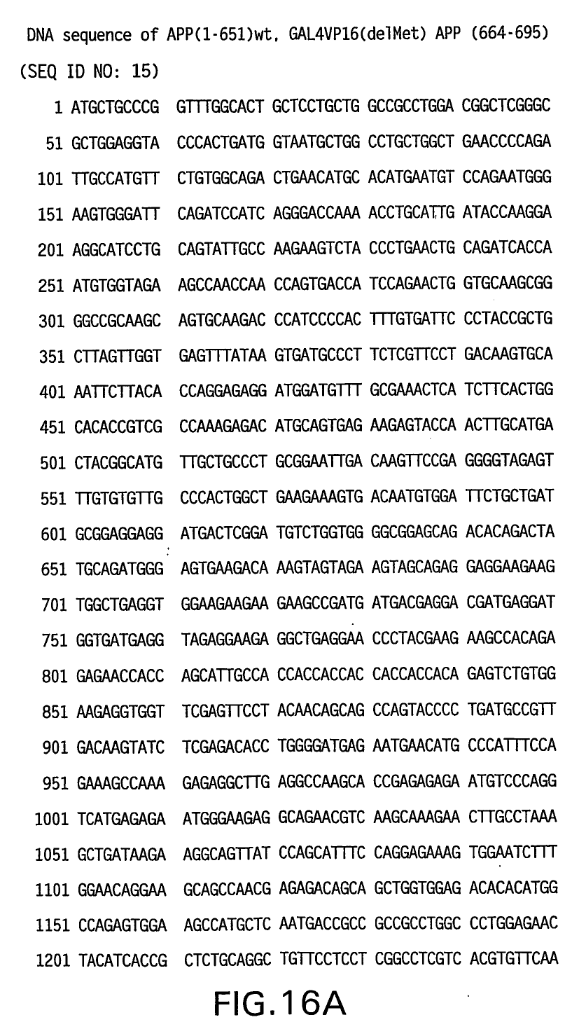

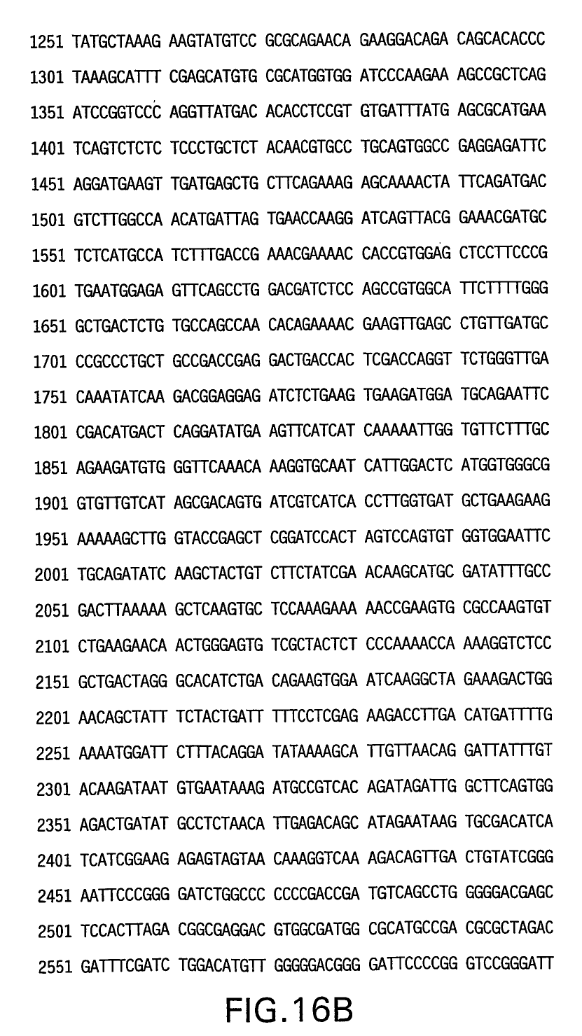

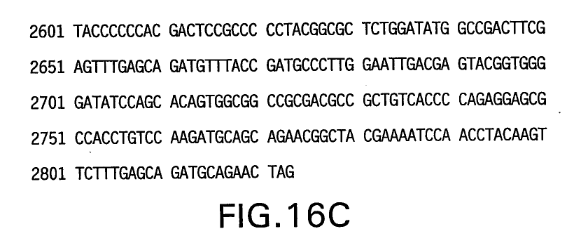

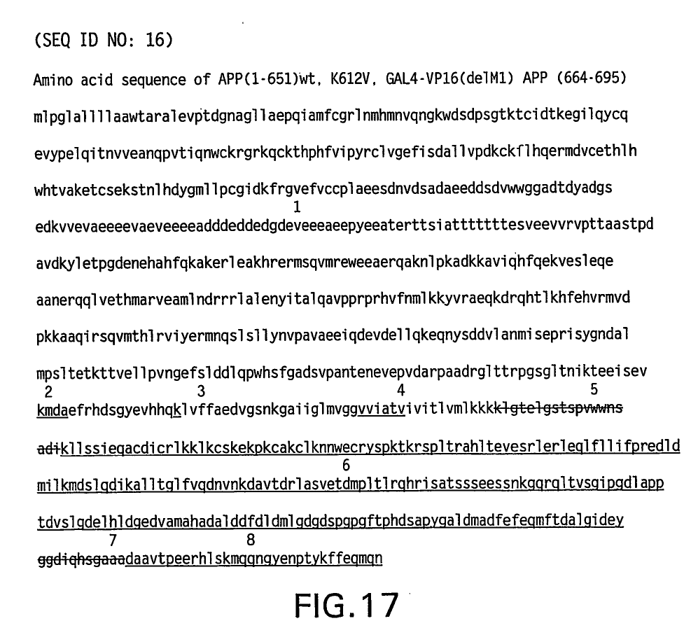

A preferred reporter gene is green fluorescent protein (GFP) or a modified GFP. Wild-type GFP has long been used in the art. Starting from green fluorescent protein, many modified versions have been derived with altered or enhanced spectral properties as compared with wild-type GFP. See, e.g., U.S. Pat. No. 5,625,048; International Patent Publication WO 97/28261; International Patent Publication WO 96/23810. Useful are the modified GFPs W1B and TOPAZ, available commercially from Aurora Biosciences Corp., San Diego, Calif. W1B contains the following changes from the wild-type GFP sequence: F64L, S65T, Y66W, N146I, M153T, and V163A (see Table 1, page 519, of Tsien, 1998, Ann. Rev. Biochem. 67:509-544). TOPAZ contains the following changes from the wild-type GFP sequence: S65G, V68L, S72A, and T203Y (see Table 1, page 519, of Tsien, 1998, Ann. Rev. Biochem. 67:509-544). Wild-type nucleotide and amino acid sequences of GFP are shown in FIG. 1 and SEQ ID NO:1 of International Patent Publication WO 97/28261; in FIG. 1 of Tsien, 1998, Ann. Rev. Biochem. 67:509-544; and in Prasher et al., 1992, Gene 111:229-233.

When expressing GFPs in mammalian cells, it may be advantageous to construct versions of the GFPs having altered codons that conform to those codons preferred by mammalian cells (Zolotukhin et al., J. Virol. 1996, 70:4646-46754; Yang et al., 1996, Nucl. Acids Res. 24:4592-4593). Another way of improving GFP expression in mammalian cells is to provide an optimal ribosome binding site by the use of an additional codon immediately after the starting methionine (Crameri et al., 1996, Nature Biotechnology 14:315-319).

Transcription factors that are useful in the present invention are preferably those transcription factors that are not naturally expressed in the recombinant host cells. This is so the regulatory DNA sequence is not activated absent APP processing and release of the transcription factor from the fusion protein. Preferably, the transcription factor contains, or is engineered to contain, a nuclear localization signal. This is so that, after release, the transcription factor will move into the nucleus of the genetically modified host cells where it can bind to, and activate, the regulatory DNA sequence, leading to expression of the reporter gene. Transcription factors, as used in the present invention, do not include proteins that, after release from a fusion protein and translocation into the nucleus, repress transcription from a reporter gene.

Among the transcription factors that are useful in the present invention are: HIV1 TAT (in particular exon I of HIV1 TAT), Gal4-VP16, the entire Gal4 protein, BIV TAT, HIV-2 TAT, SIV TAT, LexA-VP16, EBV Zta, Papillomavirus E2, or one of the bHLM homodimeric transcription factors, E12, E47, or Twist.

Expression vectors are generally used to express the fusion protein in the recombinant cells. An expression vector contains recombinant nucleic acid encoding a polypeptide (e.g., an APP/transcription factor fusion protein) along with regulatory elements for proper transcription and processing. Generally, the regulatory elements that are present in an expression vector include a transcriptional promoter, a ribosome binding site, a transcriptional terminator, and a polyadenylation signal. Other elements may include an origin of replication for autonomous replication in a host cell, a selectable marker, a limited number of useful restriction enzyme sites, and a potential for high copy number.

A variety of expression vectors are known in the art and can be used in the present invention. Commercially available expression vectors which are suitable include, but are not limited to, pMC1neo (Stratagene), pSG5 (Stratagene), pcDNAI and pcDNAIamp, pcDNA3, pcDNA3.1, pCR3.1 (Invitrogen, San Diego, Calif.), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pCI.neo (Promega), pTRE (Clontech, Palo Alto, Calif.), pV1Jneo, pIRESneo (Clontech, Palo Alto, Calif.), pCEP4 (Invitrogen, San Diego, Calif.), pSC11, and pSV2-dhfr (ATCC 37146). The choice of vector will depend upon the cell type in which it is desired to express the APP/transcription factor fusion protein, as well as on the level of expression desired, and the like.

The expression vectors can be used to transiently express or stably express the fusion protein. The transient expression or stable expression of transfected DNA is well known in the art. See, e.g., Ausubel et al., 1995, “Introduction of DNA into mammalian cells,” in Current Protocols in Molecular Biology, sections 9.5.1-9.5.6 (John Wiley & Sons, Inc.).

The recombinant host cells of the present invention are useful in methods of screening substances for the ability to inhibit APP processing. In one embodiment, the methods of the present invention comprise adding a candidate substance to a recombinant host cell comprising an APP/transcription factor fusion protein and a reporter gene and comparing the level of expression of the reporter gene protein in the presence and absence of the candidate substance, wherein the level of expression of the reporter gene protein is lower when the candidate substance inhibits processing of the APP/transcription factor fusion protein such that the transcription factor is not released, or is released in a lower amount, than in the absence of the substance.

The level of expression of the reporter gene protein is generally not measured directly. Rather, an indirect method is used. For example, fluorescence given off by the reporter gene protein may be detected or measured as, e.g., when the reporter gene product is a green fluorescent protein; or, some enzymatic activity of the reporter gene product may be detected or measured, e.g., when the reporter gene product is β-lactamase.

The candidate substance may be of any form suitable for entry into the cytoplasm of the recombinant cell or for contact with the cell's cytoplasmic membrane. Under appropriate conditions, the candidate substance may be allowed to freely diffuse into the cell, or the delivery of the substance may be facilitated by techniques and substances which enhance cell permeability, a wide variety of which are known in the art. Methods for increasing cell permeability include, without limitation, the use of organic solvents such as dimethylsulfoxide, liposomes, application of electrical current, and physical means such as substance-coated teflon pellets.

The present invention provides a method of identifying a substance that inhibits APP processing comprising:

(a) providing a recombinant eukaryotic cell which:
(i) expresses a fusion protein comprising amino acids 589-651 of APP₆₉₅ and a transcription factor where the transcription factor is fused in frame to the carboxyl terminus of amino acids 589-651 of APP₆₉₅; and
(ii) comprises a reporter gene operably linked to a regulatory DNA sequence which is capable of being activated by the transcription factor;

(b) measuring the level of reporter gene product in the cell in the absence of the substance;

(c) adding the substance to the cell and measuring the level of reporter gene product in the cell in the presence of the substance;

where a decrease in the level of reporter gene product in the presence as compared to the absence of the substance indicates that the substance inhibits APP processing.

The manner in which the level of the reporter gene product is measured will be determined by the nature of the reporter gene and, often, the characteristics of the host cell. For example, if the reporter gene product itself is fluorescent, as for example, when a green fluorescent protein is the reporter gene product, fluorescence from the cell can be measured directly. When the reporter gene product has enzymatic activity, for example, when the reporter gene product is β-lactamase, known methods of measuring that enzymatic activity can be used.

For the sake of clarity, the above method is described in terms of “a” cell. In actual practice, the method will generally be carried on a large number of cells at one time. For example, the method will often be carried out in a well of a tissue culture plate, where, depending on the number of wells in the plate (and thus their size), there can be up to hundreds, thousands, or even several million cells. The step of “adding the substance to the cell” is generally carried out by simply adding the substance to the tissue culture medium in which the cells are present. After the substance is added to the cell, the cell and the substance are incubated for a period of time sufficient for the substance to inhibit APP processing, if the substance is actually an inhibitor of APP processing. This period is usually from about 15 minutes to 48 hours, but may be somewhat more in unusual cases.

A convenient way of carrying out the method is to grow a population of the recombinant eukaryotic cells and then split the population into a portion that will be exposed to the substance and a portion that will not be exposed to the substance.

The recombinant eukaryotic cell is generally produced by transfection of an expression vector encoding the fusion protein and by transfection of a plasmid containing the reporter gene.

One skilled in the art would recognize that what is sought in terms of “a decrease in the level of reporter gene product in the presence as compared to the absence of the substance” is a non-trivial decrease. For example, if in the method described above there is found a 1% decrease, this would not indicate that the substance is an inhibitor of APP processing. Rather, one skilled in the art would attribute such a small decrease to normal experimental variance. What is looked for is a significant decrease. For the purposes of this invention, a significant decrease fulfills the usual requirements for a statistically valid measurement of a biological signal. For example, depending upon the details of the embodiment of the invention, a significant decrease might be a decrease of at least 10%, preferably at least 20%, more preferably at least 50%, and most preferably at least 90%.

In particular embodiments, amino acids 589-651 of APP₆₉₅ contain a K612V mutation.

In particular embodiments, the cell is a mammalian cell. In particular embodiments, the cell is a human cell.

In particular embodiments, the method is used to screen a library of more than 1,000 substances. In other embodiments, the method is used to screen a library of more than 50,000 substances at a rate of more than 1,000 substances per 24 hours.

In particular embodiments, the fusion protein comprises a portion of APP that is selected from the group consisting of: amino acids 1-651 of APP₆₉₅, amino acids 50-651 of APP₆₉₅, amino acids 100-651 of APP₆₉₅, amino acids 150-651 of APP₆₉₅, amino acids 200-651 of APP₆₉₅, amino acids 250-651 of APP₆₉₅, amino acids 300-651 of APP₆₉₅, amino acids 350-651 of APP₆₉₅, amino acids 400-651 of APP₆₉₅, amino acids 450-651 of APP₆₉₅, amino acids 500-651 of APP₆₉₅, and amino acids 550-651 of APP₆₉₅.

In related embodiments, the fusion protein does not comprise all of amino acids 589-651 of APP₆₉₅. Rather, the fusion protein comprises slightly fewer amino acids from APP. For example, the fusion protein might comprise slightly fewer amino acids of the β-secretase cleavage site: e.g., amino acids 590-651 of APP₆₉₅. Or the fusion protein might comprise slightly fewer amino acids of the γ-secretase cleavage site: amino acids 589-650 of APP₆₉₅; amino acids 589-649 of APP₆₉₅; amino acids 589-648 of APP₆₉₅; or amino acids 589-647. The fusion protein may even comprise slightly fewer amino acids from both ends, e.g., amino acids 590-647 of APP₆₉₅. What is important is that the portion of APP included in the fusion protein contains both the β-secretase cleavage site and the γ-secretase cleavage site.

In particular embodiments, the transcription factor is selected from the group consisting of: HIV-1 TAT, Gal4-VP16, the entire Gal4 protein, LexA-VP16, EBV Zta, Papillomavirus E2, one of the bHLH homodimeric transcription factors, including E12, E47, or Twist, or BIV TAT, BIV-2 TAT, or SIV TAT. A particular version of HIV-1 TAT suitable for use in the present invention is HIV-1 TAT exon I.

Fusion proteins suitable for use in the present invention can be selected from the group consisting of: APP(1-651)SW, K612V-TATexonI(M1L) APP (664-695) (SEQ ID NO:2); APP(1-651)wt, K612V-TATexonI(M1L) APP (664-695) (SEQ ID NO:4); APP(1-651)SW, K612V, Gal4-VP16(1L) APP (664-695) (SEQ ID NO:6); APP(1-651)wt, K612V, Gal4-VP16(M1L) APP (664-695) (SEQ ID NO:8); APP(1-651)SW, TATexonI(M1L) APP (664-695) (SEQ ID NO:10); APP(1-651)wt, TATexonI(M1L) APP (664-695) (SEQ ID NO:12); APP(1-651)SW, Gal4-VP16(M1L) APP (664-695) (SEQ ID NO:14); APP(1-651)wt, Gal4-VP16(M1L) APP (664-695) (SEQ ID NO:16); APP(1-65 1)NFEV, K612V-TATexonI(M1L) APP (664-695) (SEQ ID NO:23); and APP(1-651)NFEV, K612V, GAL4-VP16(M1L) APP (664-695) (SEQ ID NO:25).

In some embodiments of the present invention, the amino acid sequences contributed to the fusion protein by the transcription factor constitute the carboxy terminal amino acid sequences of the fusion protein. In other embodiments, the transcription factor has other sequences fused to its carboxy terminus, as in the examples herein where amino acids 664-695 of APP₆₉₅ are fused to the carboxy terminus of the transcription factor and therefore constitute the carboxy terminal amino acid sequences of the fusion protein. Other portions of APP (e.g., amino acids 652-695 of APP₆₉₅) could be used instead of amino acids 664-695 of APP₆₉₅. In fact, it should be possible to extend the carboxy terminus of the transcription factor with almost any amino acid sequences, providing such sequences do not interfere with the ability of the transcription factor to move into the nucleus and activate transcription of the reporter gene once the transcription factor has been released from the fusion protein by the action of γ-secretase.

The present invention includes a method of identifying a substance that inhibits APP processing comprising:

(a) providing a recombinant eukaryotic cell which:
(i) expresses a fusion protein comprising an amino acid sequence from APP that is capable of being cleaved by both β-secretase and γ-secretase and a transcription factor where the transcription factor is fused in frame to the amino acid sequence from APP; and
(ii) comprises a reporter gene operably linked to a regulatory DNA sequence which capable of being activated by the transcription factor;

(b) measuring the level of reporter gene product in the cell in the absence of the substance;

(c) adding the compound to the cell and measuring the level of reporter gene product in the cell in the presence of the substance;

where a decrease in the level of reporter gene product in the presence as compared to the absence of the substance indicates that the substance inhibits APP processing.

In particular embodiments, the amino acid sequence from APP comprises:

589-651 of APP₆₉₅;

590-651 of APP₆₉₅;

589-650 of APP₆₉₅;

590-650 of APP₆₉₅;

589-649 of APP₆₉₅;

590-649 of APP₆₉₅;

589-648 of APP₆₉₅;

590-648 of APP₆₉₅;

589-647 of APP₆₉₅; or

590-647 of APP₆₉₅.

In related embodiments, the amino acid sequence from APP contains the amino acid sequence NLDA (SEQ ID NO:38) at the β-secretase cleavage site instead of the wild-type sequence KMDA (SEQ ID NO:34).

In related embodiments, the amino acid sequence from APP contains the amino acid sequence NFEV (SEQ ID NO:40) at the β-secretase cleavage site instead of the wild-type sequence KMDA (SEQ ID NO:34).

The portion of the fusion protein that is derived from APP may contain mutations that are known in the art. Of particular interest are mutations that result in an increased proportion of Aβ being made in the form of Aβ1-42 rather than Aβ1-40. Such mutations are disclosed in the following publications (numbering is from the 770 amino acid version of APP):
Swedish (K670N, M671L): Mullan et al., 1992, Nature Genet. 1:345-347.
Flemish (A692G): Hendriks et al., 1992, Nature Genet. 1:218-221; Cras et al., 1998, Acta Neuropathol. (Berlin) 96:253-260.
Dutch (E693Q): Levy et al., 1990, Science 248:1124-1126.
Arctic (E693G): Nilsberth et al., 2001, Nature Neuroscience 4: 887-893.
Austrian (T714I): Kumar-Singh et al., 2000, Hum. Mol. Genet. 9:2589-2598.
French (V715M): Ancolio et al., 1999, Proc. Natl. Acad. Sci. (USA) 96:4119-4124.
Florida (I716V): Eckman et al., 1997, Hum. Mol. Genet. 6:2087-2089.
V717F: Murrell et al., 1991, Science 254:97-99.
V717G: Chartier-Harlin et al., 1991, Nature 353:844-846.
London (V717I): Goate et al., 1991, Nature 349:704-706.
L723P: Kwok et al., 2000, Ann. Neurol. 47:249-253.
I716F (also called I45F, referring to the position relative to the β-secretase cleavage site): This mutation in APP changes processing of Aβ almost exclusively to Aβ1-42.
Lichtenthaler et al., 1999, Proc. Natl. Acad. Sci. (USA) 96:3053-3058.

As with many proteins, it may be possible to modify many of the amino acids of the fusion proteins described above and still retain substantially the same biological activity in terms of APP processing as for the original fusion protein. Thus, the present invention includes modified fusion proteins which have amino acid deletions, additions, or substitutions but that still retain substantially the same properties with respect to APP processing as the fusion proteins described herein. It is generally accepted that single amino acid substitutions do not usually alter the biological activity of a protein (see, e.g., Molecular Biology of the Gene, Watson et al., 1987, Fourth Ed., The Benjamin/Cummings Publishing Co., Inc., page 226; and Cunningham & Wells, 1989, Science 244:1081-1085). Accordingly, the present invention includes fusion proteins where one amino acid substitution has been made in the fusion proteins described herein where the fusion proteins still retain substantially the same properties with respect to APP processing as the fusion proteins described herein. The present invention also includes fusion proteins where two or more amino acid substitutions have been made in the fusion proteins described herein where the fusion proteins still retain substantially the same properties with respect to APP processing as the fusion proteins described herein. In particular, the present invention includes embodiments where the substitutions are conservative substitutions.

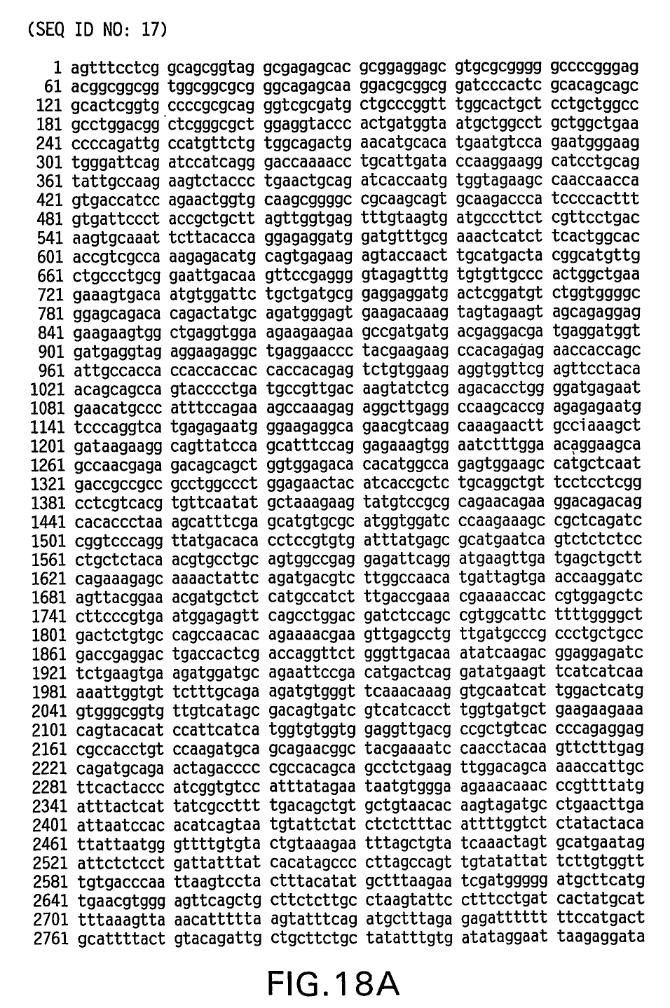

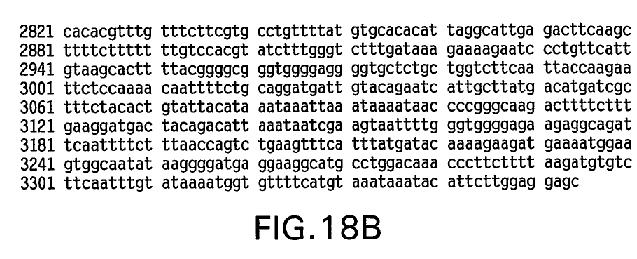

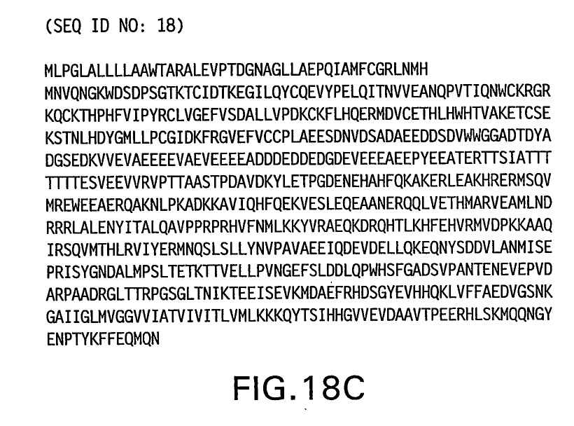

With the exception of FIG. 18, the nucleotide and amino acid sequences of APP disclosed herein contain a minor difference compared to APP sequences that are usually reported in the literature. For the sequences disclosed herein with such a difference, the nucleotide at position 367 is an A rather than a G, as in most published APP sequences. This change results in a conservative substitution in the corresponding APP amino acid sequence. Thus, the amino acid sequences disclosed herein with such a difference have an I rather than a V at position 123. This difference does not affect the properties of the fusion proteins for the purposes of the present invention. Therefore, fusion proteins having the APP sequence reported in the literature with an G at nucleotide position 367 and a V at amino acid position 123 and the fusion proteins disclosed herein with an A at nucleotide position 367 and an I at amino acid position 123 are to be considered equivalents for the purposes of the present invention.

The Gal-VP16 sequences disclosed herein contain two changes from the usual published sequences. There is T to C change at nucleotide position 2131 that causes a S to P change at amino acid position 712; there is A to C change at nucleotide position 2301 that does not change the amino acid sequence. It is expected that Gal-VP16 proteins containing the usual sequences reported in the literature will also be suitable for use in the present invention.

The methods of the present invention can be used to screen libraries of substances or other sources of substances to identify substances that are inhibitors of β-secretase or γ-secretase. Such identified inhibitory substances can serve as “leads” for the development of pharmaceuticals that can be used to treat patients having Alzheimer's disease or in a prophylactic manner to prevent or delay the development of Alzheimer's disease. Such leads can be further developed into pharmaceuticals by, for example, subjecting the leads to sequential modifications, molecular modeling, and other routine procedures employed in the pharmaceutical industry. The inhibitors of APP processing identified by the present invention may also be tested in animal models of Alzheimer's disease such as the various transgenic mouse models that are known in the art.

Although a wide variety of substances can be screened by the methods of the present invention, preferred substances for screening are libraries of small molecule compounds. Small molecule compounds are preferred because they are more readily absorbed after oral administration, have fewer potential antigenic determinants, and are more likely to cross the blood/brain barrier than larger molecules such as nucleic acids or proteins.

Once identified by the methods of the present invention, the candidate small molecule compounds may then be produced in quantities sufficient for pharmaceutical testing and formulated in a pharmaceutically acceptable carrier (see, e.g., Remington's Pharmaceutical Sciences, Gennaro, A., ed., Mack Publishing, 1990, for suitable methods). The candidate compounds may be administered to cell lines relevant to Alzheimer's disease, animal models of Alzheimer's disease, or Alzheimer's disease patients.

The numbering of the amino acids in APP used herein is based on the 695 amino acid version of APP described in Kang et al., 1987, Nature 325:733-736. There are two other major versions of APP, having 751 amino acids and 770 amino acids (see, Ponte et al., 1988, Nature 331:525-527 for the 751 amino acid version and Kitaguchi et al., 1988, Nature 331:530-532 for the 770 amino acid version). One skilled in the art will understand how to translate the numbering used herein, based on the 695 amino acid version of APP, into the corresponding numbering for other versions of APP. For example, some of the APP/transcription factor fusion proteins of the present invention contain the K612V mutation, based on the numbering of the 695 amino acid version. This mutation would correspond to a K668V mutation in the 751 amino acid version and a K687V mutation in the 770 amino acid version.

Therefore, when a “K612V” mutation is referred to herein, it will be understood that such reference also includes a K668V mutation of the 751 amino acid version of APP as well as a K687V mutation of the 770 amino acid version of APP.

Similarly, the portion of APP referred to as APP₁₋₆₅₁ herein, based on the 695 amino acid version, will be understood to mean also APP₁₋₇₀₇ of the 751 amino acid version and APP₁₋₇₂₆ of the 770 amino acid version.

If desired, inhibitors that are identified by the methods of the present invention can be further tested to determine which step in APP processing they affect. Assays that are known to be specific for the various steps of APP processing can be used for this purpose. For example, the assay of Karlström et al., (Journal of Biological Chemistry papers in press, published on Dec. 13, 2001 as Manuscript C100649200) is only capable of detecting inhibitors of γ-secretase and cannot also detect inhibitors of other steps of APP processing such as, e.g., inhibitors of β-secretase. If an inhibitor identified by the methods of the present invention is found to also be an inhibitor when tested in the assay of Karlström et al., then that inhibitor is at least a γ-secretase inhibitor. It is still possible that that inhibitor could inhibit other steps in APP processing as well. Further tests known in the art can determine this.

The present invention may be modified so as to provide methods of determining at which step of APP processing a known inhibitor of APP processing exerts its effect. The known inhibitor may be one that has been identified by the methods of the present invention or by some other method. The modification to the present invention consists in mutating the β-secretase site in a fusion protein so that β-secretase cleavage can no longer occur at the site or occurs at a very much reduced level. Providing that the fusion protein contains a cleavable α-secretase site, the fusion protein can still be used in the methods of the present invention. However, this fusion protein (with a mutated β-secretase site) can no longer detect β-secretase inhibitors. Therefore, if the known APP processing inhibitor still functions as an APP processing inhibitor in this modified version of the invention, then the known inhibitor cannot be a β-secretase site inhibitor but instead must exert its effect downstream of β-secretase.

Suitable mutations of the β-secretase site include the following. All the sequences are for amino acid positions 594-598 of APP₆₉₅. VNFAV (SEQ ID NO:41): This mutation shows decreased β-secretase cleavage relative to the wild type, KMDA (SEQ ID NO:34), sequence. VKVDA (SEQ ID NO:42): Vassar et al., 1999, Science 286:735-741. This mutant was tested in vitro only, but purified β-secretase failed to cleave a 30-amino acid peptide containing this sequence.

WKMDA (SEQ ID NO:43), VKADA (SEQ ID NO:44), VKKDA (SEQ D) NO:45), VKEDA (SEQ ID NO:46), VKIDA (SEQ ID NO:47), VKMIA (SEQ ID NO:48), VKMNA (SEQ ID NO:49), VKUEA (SEQ ID NO:50), VKMDE (SEQ ID NO:51), VKMDK (SEQ ID NO:52): Citron et al., 1995. Neuron 14:661-670. These mutations decreased Aβ production 4-20× relative to p3 production in cultured cells.

Fusion proteins can be constructed by use of the polymerase chain reaction (PCR) to amplify desired portions of APP and transcription factors, which can be then be cloned into expression vectors by methods well known in the art. Primers for PCR will generally include a small part of the APP or transcription factor as well as convenient cloning sites and/or linker peptide sequences. The PCR primers can be used to amplify the desired APP or transcription factor fragments from sources such as previously cloned APP or transcription factors, cDNA libraries, or genomic libraries. The amplified APP and transcription factor sequences can be cloned into suitable expression vectors. Methods of PCR and cloning are well known in the art and can be found in standard reference materials such as those listed below.

Standard techniques for cloning, DNA isolation, amplification and purification, for enzymatic reactions involving DNA ligase, DNA polymerase, restriction endonucleases and the like, and various separation techniques are known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N. Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York, and Ausubel et al. (1992) Current Protocols in Molecular Biology, Greene/Wiley, New York, N.Y.

PCR reactions can be carried out with a variety of thermostable enzymes including but not limited to AmpliTaq, AmpliTaq Gold, or Vent polymerase. For AmpliTaq, reactions can be carried out in 10 mM Tris-Cl, pH 8.3, 2.0 mM MgCl₂, 200 μM of each dNTP, 50 mM KCl, 0.2 μM of each primer, 10 ng of DNA template, 0.05 units/μl of AmpliTaq. The reactions are heated at 95° C. for 3 minutes and then cycled 35 times using suitable cycling parameters, including, but not limited to, 95° C., 20 seconds, 62° C., 20 seconds, 72° C., 3 minutes. In addition to these conditions, a variety of suitable PCR protocols can be found in PCR Primer, A Laboratory Manual, edited by C. W. Dieffenbach and G. S. Dveksler, 1995, Cold Spring Harbor Laboratory Press; or PCR Protocols: A Guide to Methods and Applications, Michael et al., eds., 1990, Academic Press.

It is desirable to sequence the DNA encoding the fusion proteins, or at least the junction regions of the various portions (APP, transcription factor, linkers) of the fusion protein in order to verify that the desired portions have in fact been obtained, joined properly, and that no unexpected changes have been introduced into the sequences by the PCR reactions.

Suitable PCR primers for amplification of DNA sequences for use in the present invention can be readily designed by those of skill in the art. Examples of such primers are shown below.
5′-GGA GAG GAT ATC ATG GAG CCA GTA GAT CC-3′ (SEQ ID NO:53) can be used to amplify the 5′ portion of HIV-1 TAT exon I.
5′-TAC ATG GCG GCC GCC TAC TTA CTG CTT TG-3′ (SEQ ID NO:54) can be used to amplify the 3′ portion of HIV-1 TAT exon I.
5′-GGA TGT GAT ATC TTT CTT CTT CAG CAT CAC CAA GG-3′ (SEQ ID NO:55) can be used to amplify the 3′ portion of DNA encoding amino acids 1-651 of APP, i.e., the transmembrane region of APP.

The following non-limiting examples are presented to better illustrate the invention.

### EXAMPLE 1

### Transfection of pcDNA3.1 zeo (+) APP(1-651)SW, K612V-(M1L)TATexonI With pMM321

The following example demonstrated that an APP/TAT fusion construct will transactivate a reporter gene in which the HIV1 LTR regulatory DNA sequence controls the expression of enhanced green fluorescent protein (EGFP). The following also serves as an example of the kind of preliminary routine variations of fusion protein levels and inhibitor levels that may be advantageous to test in the practice of the present invention. Such routine variations are often helpful in validating the assays before a large scale screening project is undertaken.

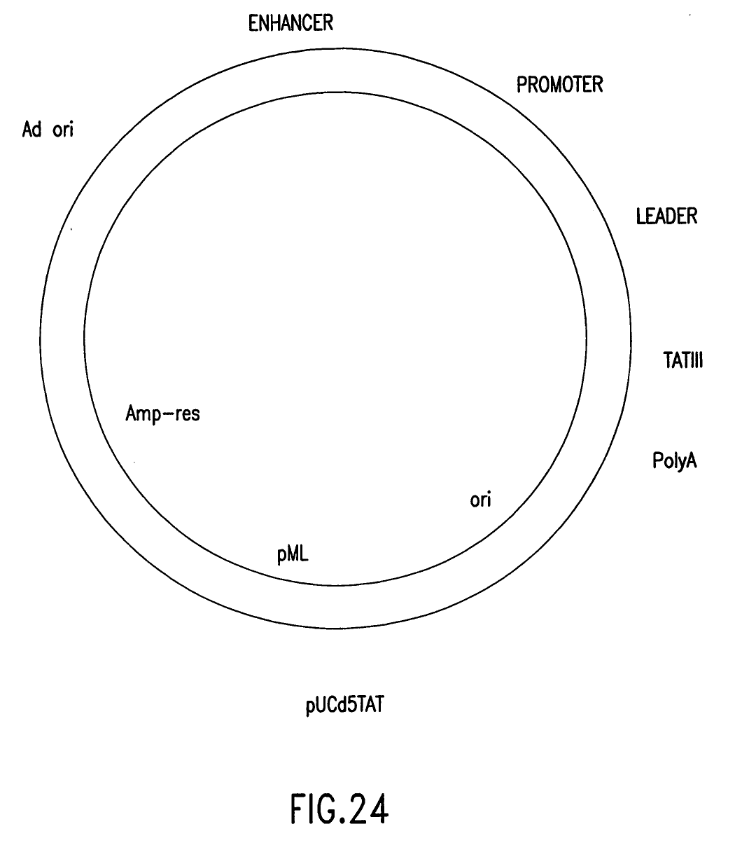

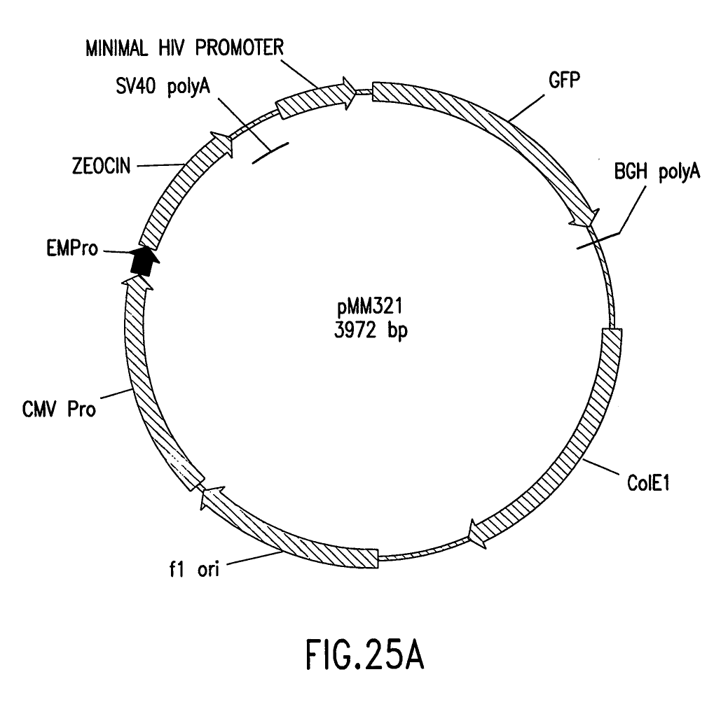

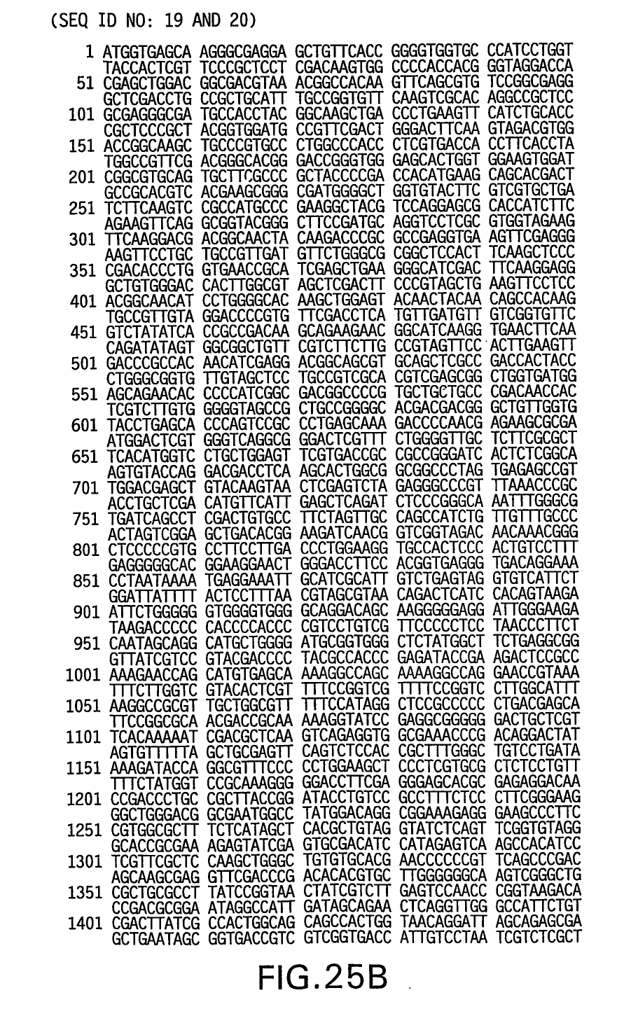

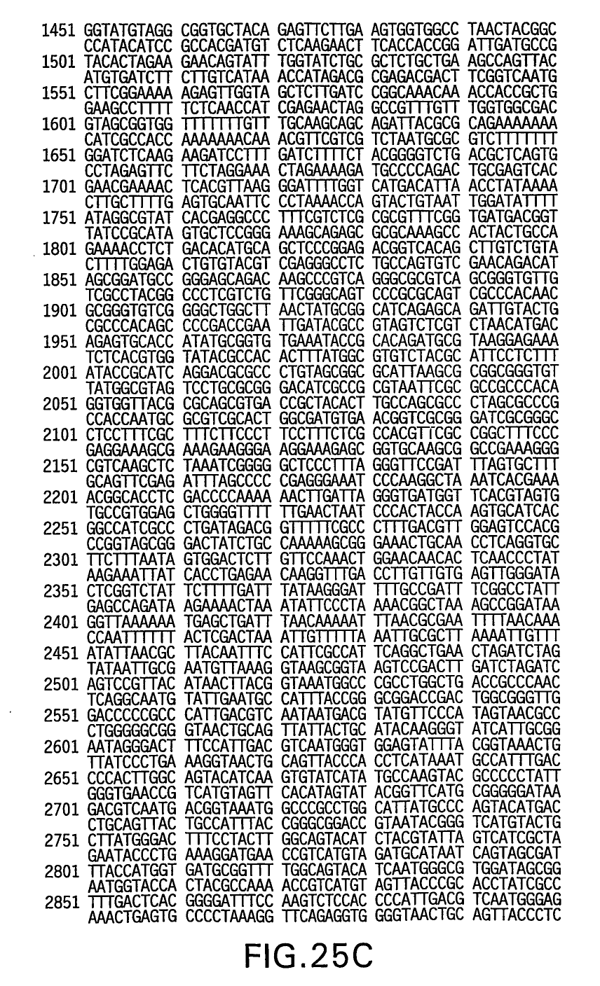

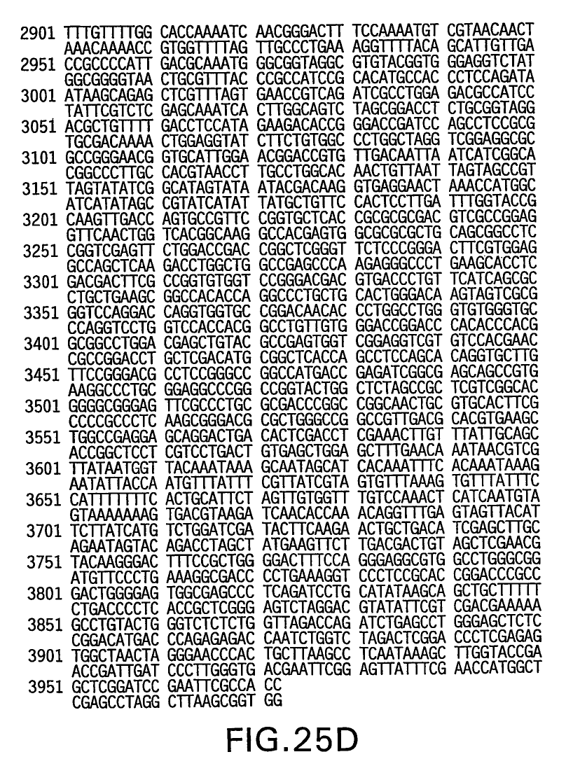

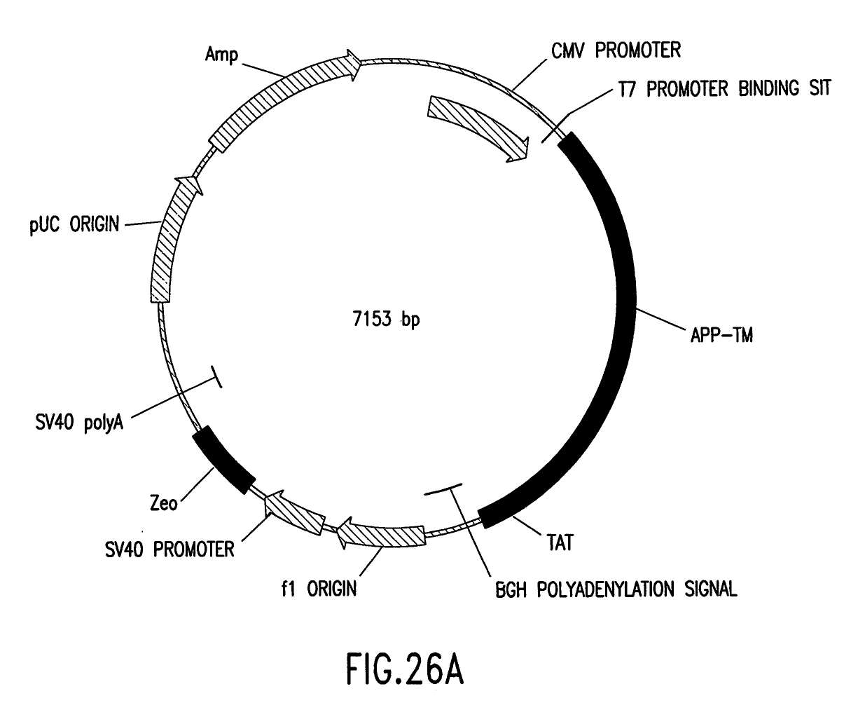

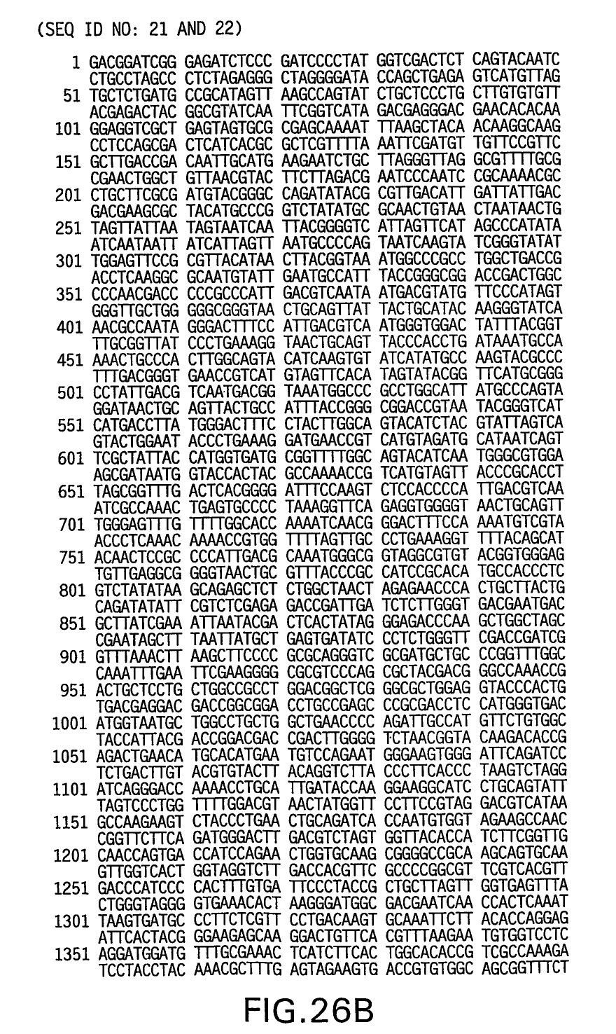

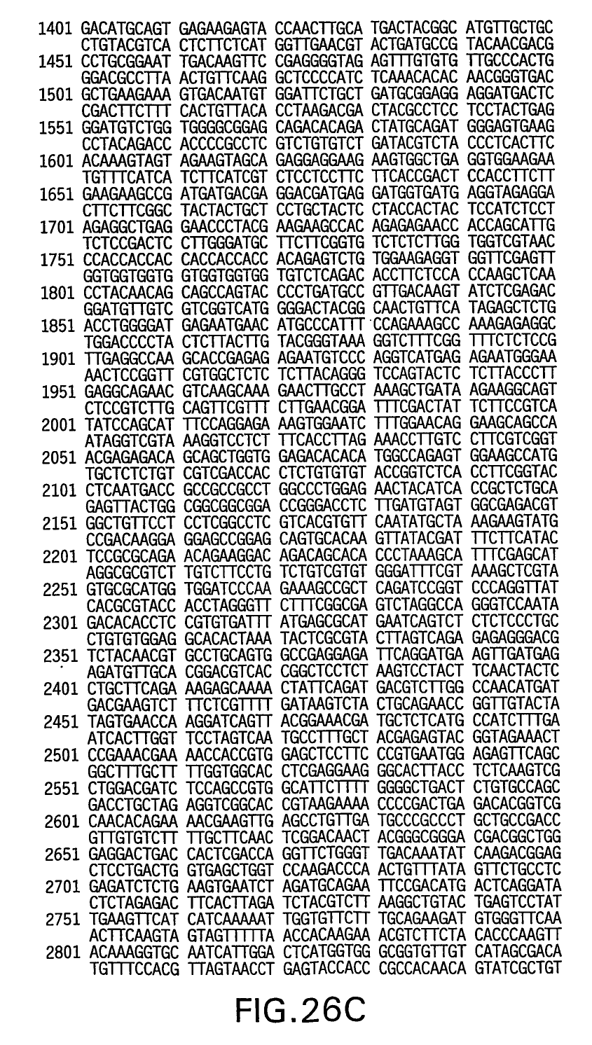

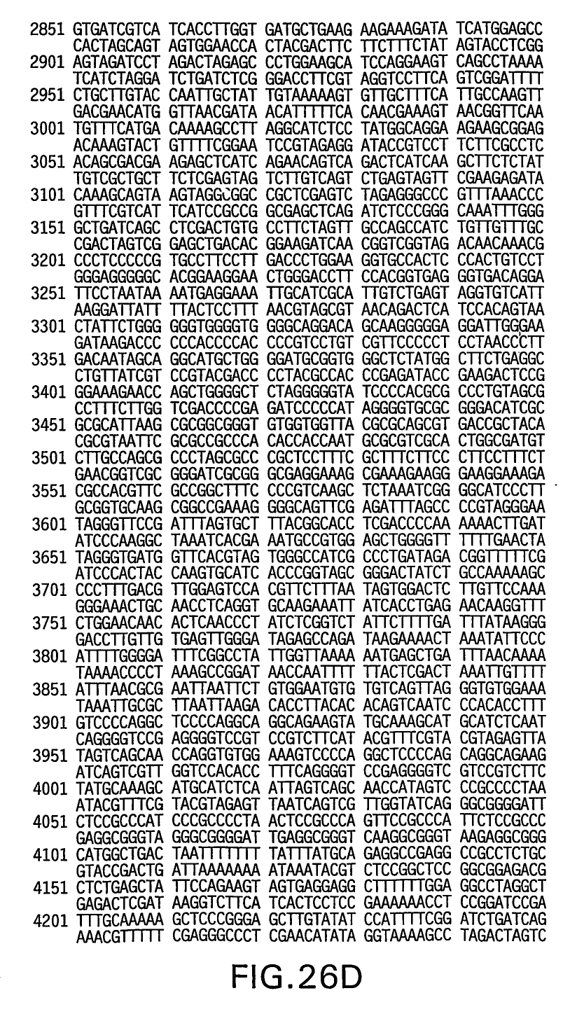

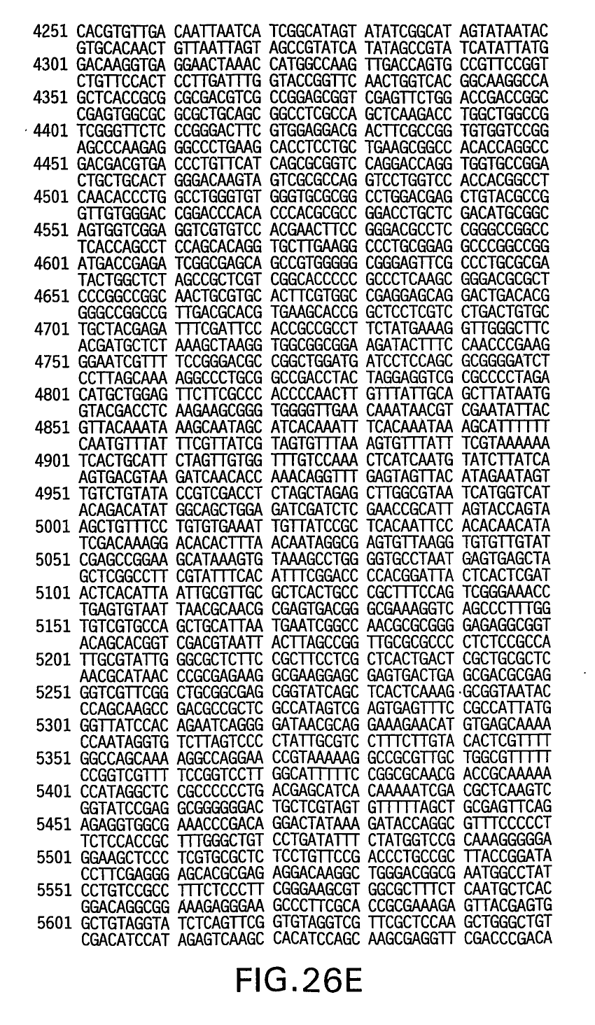

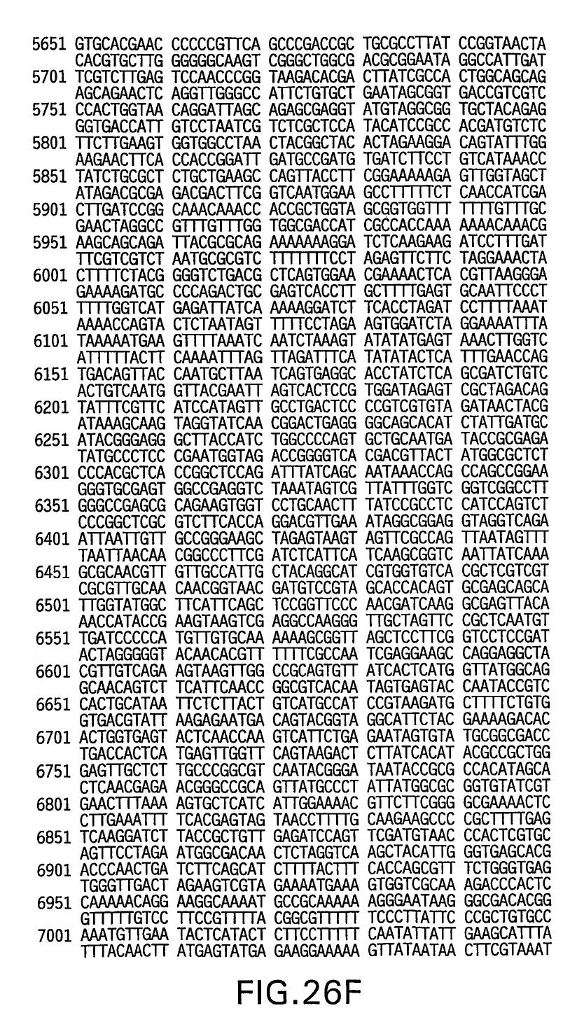

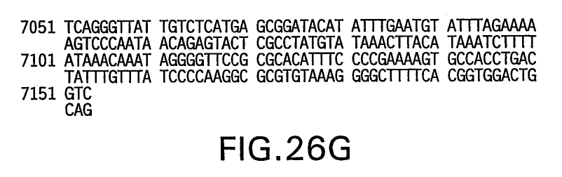

The APP/TAT fusion construct is referred to as “pcDNA3.1 zeo (+) APP(1-651)SW, K612V-(M1L)TATexonI” (see FIG. 26) and contains the HIV1 TAT exon 1 fused just after the transmembrane domain of APP. This construct is also shown in outline form in FIG. 1B. “pMM321” refers to a reporter gene plasmid consisting of the HIV1 LTR driving the transcription of enhanced green fluorescent protein (see FIG. 25). As a positive control for TAT expression, a construct in which TAT was under the control of a strong, constitutive promoter ( referred to as “pUCd5TAT”; see FIG. 24) was used.

Methods:

1. Day 1: Pass HEK 293T cells into 2×6 well dishes at 1×10⁵ cells/well.
2. Day 2: Transfect cells with 9 μL Fugene and 0.125 μg pMM321 and various amounts of pcDNA3.1 zeo (+) APP(1-651)SW, K612V-(M1L)TATexonI.
   Plate 1

1. 5 μg pcDNA3.1 zeo (+) APP(1-651)SW, K612V-(M1L)TATexonI

2. 2.5 μg pcDNA3.1 zeo (+) APP(1-651)SW, K612V-(M1L)TATexonI

3. 1.25 μg pcDNA3.1 zeo (+) APP(1-651)SW, K612V-(M1L)TATexonI

4. 0.625 μg pcDNA3.1 zeo (+) APP(1-651)SW, K612V-(M1L)TATexonI

5. 0.312 μg pcDNA3.1 zeo (+) APP(1-651)SW, K612V-(M1L)TATexonI

6. 0.156 μg pcDNA3.1 zeo (+) APP(1-651)SW, K612V-(M1L)TATexonI

Plate 2:

1. 0.08 μg pcDNA3.1 zeo (+) APP(1-651)SW, K612V-(M1L)TATexonI

2. 0.04 μg pcDNA3.1 zeo (+) APP(1-651)SW, K612V-(M1L)TATexonI

3. no pcDNA3.1 zeo (+) APP(1-651)SW, K612V-(M1L)TATexonI

4. 0.625 pUCd5TAT

5. 0.625 μg pcDNA3.1 zeo (+) APP(1-651)SW, K612V-(M1L)TATexonI and 1 μg pMM321

6. 1 μg pMM321

Six hours post-transfection, green cells were only observed in plate 2, #5.
3. Day 3: The fluorescence intensity of the transfected cells was observed and recorded.
4. Day 4: The fluorescence intensity of transfected cells was observed and recorded.
   Results:

Co-transfection with pcDNA3.1 zeo (+) APP(1-651)SW, K612V-(M1L)TATexonI increased GFP expression in the cells.

Day 3:

5 μg—no green cells

2.5 μg—no green cells (too much DNA for these two transfections?)

1.25 μg—many bright and dim green cells (see photographs and figure in ancillary data)

0.625 μg—bright and dim green cells but fewer than at 1.25 μg

0.312 μg—no difference obvious between 0.625 μg and 0.312 μg

0.156 μg—very few green cells

0.08 μg—very few green cells

0.04 μg—very few green cells

no pcDNA3.1 zeo (+) APP(1-651)SW, K612V-(M1L)TATexonI—extremely few (if any) green cells

0.625 μg pUCd5TAT—cells were extremely bright, not necessarily more in number than with pcDNA3.1 zeo (+) APP(1-651)SW, K612V-(M1L)TATexonI

0.625 μg pcDNA3.1 zeo (+) APP(1-651)SW, K612V-(M1L)TATexonI +1 μg pMM321—many bright and dim green cells.

1 μg pMM321 many-fold fewer green cells, some bright, most dim.

Day 3—changed media (saved 1 mL conditioned media from wells Plate 1-3, 4, 5, 6; Plate 2-1, 2, 3, 5, 6). Added fresh media with 10 μM of L-685,458 (a potent, cell permeable γ-secretase inhibitor) to wells Plate 1-3, 4, 5, 6; Plate 2-3, 4, 5, 6. Waited 48 hours to observe loss of fluorescence since GFP is so stable.

After 48 hours, all wells appeared brighter than at 24 hour time point. This does not necessarily mean that the inhibitor was ineffective, or that the assay did not work, since there were no controls run where the inhibitor was not added. However, it does suggest that under these conditions it may be preferable to add the inhibitor at the time of transfection to shut down γ-secretase as soon as possible and avoid release of TAT and induction of GFP.

### EXAMPLE 2

### Transfection of APP(1-651)SW, K612V-(MIL)TATexonI Into HEK293T and H4 Cells Accompanied By Inhibition of γ-secretase Activity With L-685,458

The following example demonstrates the operation of the invention in HEK293T cells and H4 cells and shows inhibition of APP processing (and thus TAT release) by treatment with a known γ-secretase inhibitor. “pcDNA3.1 zeo (+) APP(1-651)SW, K612V-(M1L)TATexonI,” “pMM321,” and “pUCd5TAT” are the same as in Example 1. H4 cells (ATCC HTB-148) are a neuronal cell line.

Methods:

Day 1: Plated out 2×6 well plates of HEK293T cells and 2×6 well plates of H4 cells at 1×10⁵ cells/well.
Day 2: Transfected cells with 2 μg total DNA—pcDNA3.1 zeo (+) APP(1-651)SW, K612V-(M1L)TATexonI and carrier (a pET-IN plasmid).
   Plate 1:

1,2: 1 μg pMM321+1 μg pcDNA3.1 zeo (+) APP(1-651)SW, K612V-(M1L)TATexonI

3,4: 1 μg pMM321+0.1 μg pcDNA3.1 zeo (+) APP(1-651)SW, K612V-(M1L)TATexonI+0.9 μg carrier

5: 1 μg pMM321+1 μg carrier (added too much carrier to this well in H4 cells)

6: 1 μg pMM321+0.1 μg pUCd5TAT+0.9 μg carrier

Plate 2:

1,2: 0.1 μg pMM321+1 μg pcDNA3.1 zeo (+) APP(1-651)SW, K612V-(M1L)TATexonI+0.9 μg carrier

3,4: 0.1 μg pMM321+0.1 μg pcDNA3.1 zeo (+) APP(1-651)SW, K612V-(M1L)TATexonI+1.8 μg carrier (added too 0.5×carrier to this mix in 293T cells)

5: 0.1 μg pMM321+1.9 μg carrier

6. 0.1 μg pMM321+0.1 μg pUCd5TAT+1.8 μg carrier

Transfections for HEK293T cells: 9 μL Fugene/well. Combined with DNA in Optimem and incubated and added to cells according to manufacturer's instructions.

Transfections for H4 cells: 6 μL Fugene/well. Combined with DNA in Optimem and incubated and added to cells according to manufacturer's instructions.

Added 10 μM L-685,458 to Plates 1 and 2, wells 2 and 4 for both cell types within 1 hour of transfection. Observed cells periodically.

Took pictures at 24, 46 hours after transfection, using AE lock to keep exposures constant between wells.

Results:

Both H4 and 293T cells turned much brighter green in the presence of pcDNA3.1 zeo (+) APP(1-65 1)SW, K612V-(M1L)TATexonI
   At 24 hours:
   H4 cells:
   Plate 1: 1 μg pMM321

1. 1 ug pMM321+1 ug pcDNA3.1 zeo (+) APP(1-651)SW, K612V-(M1L)TATexonI: Many bright and dim green cells (good transfection efficiency as well)

2. 1 ug pMM321+1 ug pcDNA3.1 zeo (+) APP(1-651)SW, K612V-(M1L)TATexonI+10 μM L-685,458: Also many bright and dim green cells, but reduced compared with well #1

3. Very few green cells (a few per field)

4. Very few green cells

5. A few dimly green cells

6. Some induction with 0.1 μg pUCd5TAT but still relatively few cells.

Plate 2: 0.1 μg pMM321

1. 0.1 μg pMM321+1 μg pcDNA3.1 zeo (+) APP(1-651)SW, K612V-(M1L)TATexonI: ˜10 bright green cells/field and the rest are dim green

2. 0.1 μg pMM321+1 μg pcDNA3.1 zeo (+) APP(1-651)SW, K612V-(M1L)TATexonI+10 μM L-685,458: ˜3-5 bright green cells/field, some dim green, and some not green.

3. No green cells

4. No green cells

5. No green cells

6. A few bright green cells

HEK293T Cells:

Plate 1: 1μg pMM321

1. +1 μg pcDNA3.1 zeo (+) APP(1-651)SW, K612V-(M1L)TATexonI: of 15 cells: 6 dim, 4 medium, 5 bright

2. +1 μg pcDNA3.1 zeo (+) APP(1-651)SW, K612V-(M1L)TATexonI+10 μM L-685,458: of 15 cells: 6 very dim, 5 dim, 1 medium, 3 bright

3. +0.1 μg pcDNA3.1 zeo (+) APP(1-651)SW, K612V-(M1L)TATexonI: many more green cells than 1 μg, lots of strong, bright green cells

4. +0.1 μg pcDNA3.1 zeo (+) APP(1-651)SW, K612V-(M1L)TATexonI+10 μM L-685,458: Fewer bright green cells/field but intensity does not appear strongly diminished

5. 1 μg pMM321 alone: Most cells in the field expressing dim to medium levels of GFP

6. enhancement by 0.1 μg pUCd5TAT

Plate 2: 0.1 μg pMM321

1. +1 μg pcDNA3.1 zeo (+) APP(1-651)SW, K612V-(M1L)TATexonI: Bright and medium green cells

2. +1 μg pcDNA3.1 zeo (+) APP(1-651)SW, K612V-(M1L)TATexonI+10 μM L-685,458: Bright, medium, and dim green cells

3. +0.1 μg pcDNA3.1 zeo (+) APP(1-651)SW, K612V-(1L)TATexonI: Bright, medium, and dim green cells

4. +0.1 μg pcDNA3.1 zeo (+) APP(1-651)SW, K612V-(41L)TATexonI+10 μM L-685,458: Bright, medium, and dim green cells

5. 0.1 μg pMM321 alone: Most expressing cells have dim GFP, a few medium to bright cells

6. Enhancement by 0.1 μg pUCd5TAT

Changed media on cells at 24 hours past transfection. Kept 10 μM L-685,458 on cells in wells 2 and 4.

At 46 hours after transfection, examined the wells again. Lots of floating cells in all wells, all cell types. Highest number of floaters in 1 μg pcDNA3.1 zeo (+) APP(1-651)SW, K612V-(M1L)TATexonI lanes.

Took some photographs under fluorescent and white light (white light at low intensity) to reveal fluorescent and non-fluorescent cells. Conducted a subjective analysis of the photographs to see if the amount of inhibition by 10 μL-685,458 was in any way quantifiable. Counted bright (white in the middle), strong (blue middle), medium (green) and dim/non-fluorescent cells and determined the approximate fraction of each level of expression. Results follow:

TABLE 1
293T cells transfected with X μg pMM321 (first number in left column) and X μg
pcDNA3.1 zeo (+) APP(1-651)SW, K612V-(M1L)TATexonI (second number in left column)

| Transfection | # Bright | # Strong | # Med | # Non | % Bright | % Strong | % Med | % Non |
|---|---|---|---|---|---|---|---|---|
| 1 μg + 1 μg | none | 8 | 81 | 316 | | 2 | 20 | 78 |
| 1 + 1 + cmpd | none | 5 | 66 | 521 | | 0.8 | 11 | 88 |
| 1 + 0.1 | 41 | 77 | 143 | 61 | 12 | 24 | 44 | 19 |
| 1 + 0.1 + cmpd | 23 | 32 | 149 | 194 | | | | |

The results shown in Table 1 indicate that the presence of L-685,458 (“cmpd”) caused fewer strong and medium fluorescing cells as well as more non-fluorescent cells in the first run; in the second run, L-685,458 caused fewer bright and strong fluorescing cells as well as more non-fluorescent cells (with slightly more medium fluorescing cells). Overall, these data clearly indicate that the presence of an inhibitor of APP processing such as L-685,458 can be identified by the present invention.

1 mL of conditioned media from each well was analyzed for production of Aβ. Higher than background levels of Aβ were observed in 293T cells after transfection with 1 μg pcDNA3.1 zeo (+) APP(1-651)SW, K612V-(M1L)TATexonI and 0.1 μg pcDNA3.1 zeo (+) APP(1-651)SW, K612V-(M1L)TATexonI and higher than background levels of Aβ in H4 cells after transfection with 1 μg pcDNA3.1 zeo (+) APP(1-651)SW, K612V-(M1L)TATexonI, but not 0.1 μg pcDNA3.1 zeo (+) APP(1-651)SW, K612V-(M1L)TATexonI (no enhancement of GFP was observed with 0.1 μg pcDNA3.1 zeo (+) APP(1-651)SW, K612V-(M1L)TATexonI either). Aβ was completely inhibited to background levels by 10 μM L-685,458. Surprisingly, substantial inhibition of GFP was not observed with 10 μM L-685,458.

100,000 cells from each well were trypsinized and placed in 0.1 mL phenol red-free media in a Costar 96-well dish and read using the fluorometer. The results are shown below:

TABLE 2

| | | 1 ug | 10 uM | 0.1 ug | 10 uM | | 0.1 ug |
|---|---|---|---|---|---|---|---|
| | | APPTAT | 458 | APPtat | 458 | no tat | pUCd5TAT |
| 293T | 1 ug LTRGFP | 17670 | 14321 | 65535 | 65535 | 14890 | 65535 |
| | 0.1 ug LTRGFP | 9976 | 10491 | 17677 | 14790 | 9624 | 25735 |
| H4 | 1 ug LTRGFP | 21307 | 25307 | 7175 | 7136 | 7147 | 7277 |
| | 0.1 ug LTRGFP | 9574 | 10031 | 7317 | 6957 | 6946 | 7247 |
| | Blank | 7498 | 7124 | 7570 | 7454 | 5774 | 7638 |

In Table 2, “APPtat” is pcDNA3.1 zeo (+) APP(1-651)SW, K612V-(M1L)TATexonI. “458” is L-685,458. “LTRGFP” is pMM321.

0.1 μg pcDNA3.1 zeo (+) APP(1-651)SW, K612V-(M1L)TATexonI with and without compound exceeded the maximum reading of the fluorometer, as did the addition of 0.1 μg pUCd5TAT to cells transfected with 1 μg pMM321.

1 μg pcDNA3.1 zeo (+) APP(1-651)SW, K612V-(M1L)TATexonI+1 μg pMM321 incrementally increased the amount of fluorescence relative to 1 μg pMM321 alone, and this was reduced to background levels by 10 μM L-685,458. Inhibition of fluorescence was also observed in 293T cells transfected with 0.1 μg pcDNA3.1 zeo (+) APP(1-651)SW, K612V-(M1L)TATexonI+0.1 μg pMM321. No inhibition of fluorescence was observed in H4 cells under any transfection conditions.

### EXAMPLE 3

### Use of APP(1-651)SW, K612V-TATexonI in H4 Cells

L-875,532 is a known γ-secretase inhibitor having the structure shown below. It is described and details of its synthesis are disclosed in Seiffert et al., 2000, J. Biol. Chem. 275:34086-34091.

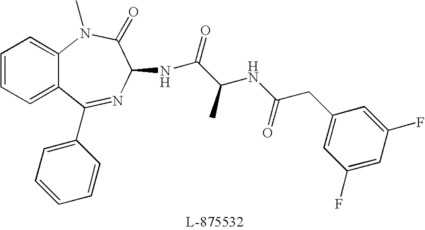

Compound X is a β-secretase inhibitor.

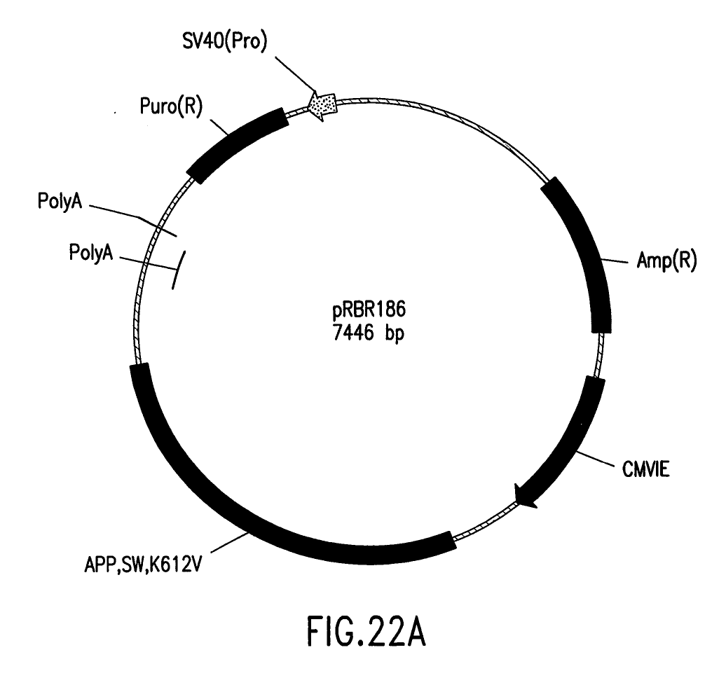

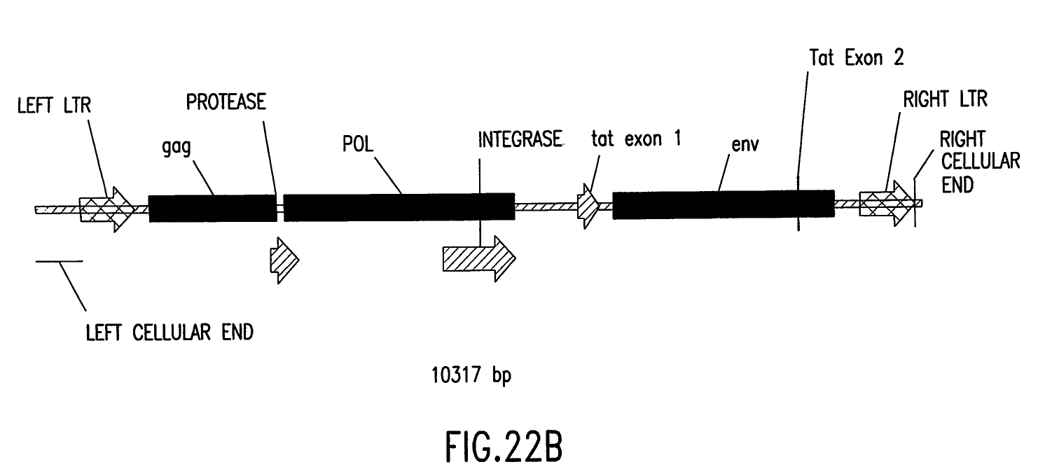

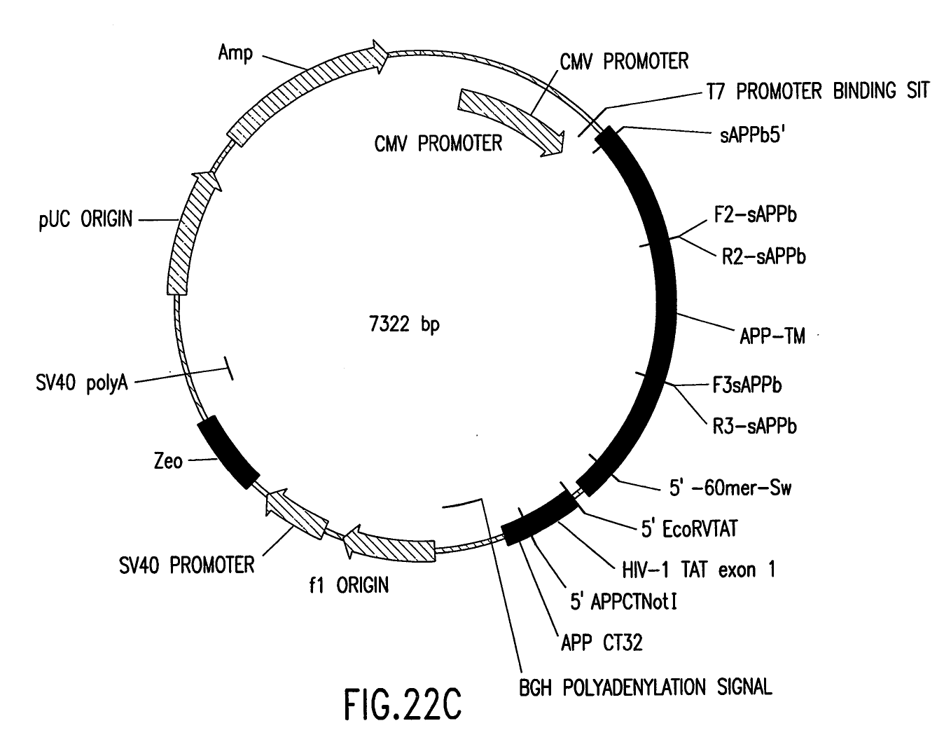

pRBR186 (FIG. 22A) is an expression vector containing DNA sequences encoding full-length APP containing the Swedish mutation and the K612V mutation. pRBR186 does not contain a transcription factor fused to the APP sequences.

pcDNA3.1 zeo (+) APP(1-651)SW, K612V-(M1L)TATexonI is an expression vector that directs the expression of a the fusion protein APP(1-651)SW, K612V-(M1L)TATexonI in mammalian cells. This fusion protein contains the first 651 amino acids of APP (with a Swedish version of the β-secretase cleavage site as well as the K612V mutation) fused in frame to exon I of HIV1 TAT, which has been modified with a Met1-Leu mutation. A schematic diagram of pcDNA3.1 zeo (+) APP(1-651)SW, K612V-(M1L)TATexonI is shown in FIG. 26A. The nucleotide sequence of pcDNA3.1 zeo (+) APP(1-651)SW, K612V-(M1L)TATexonI (SEQ ID NO:22) is shown in FIG. 26B-G.

Methods:

1. H4 cells (ATCC HTB-148) were transfected with the various constructs listed below using 6 μL Fugene per 100 μL Optimem and 100 μL Optimem per well (6-well dishes). Transfection reactions were incubated for 30 minutes prior to adding 100 μL dropwise onto wells.

Transfections were done as follows:

1. 1 μg pMM321 (FIG. 25A-D) and 1 μg pcDNA3.1 backbone

1a. 1 μg pMM321 and 1 μg pcDNA3.1 (Invitrogen, San Diego, Calif.) backbone. Prior to transfection, 10 μM L-875,532 (γ-secretase inhibitor) was added to the well.

2. 1 μg pMM321 and 1 μg pRBR186 (FIG. 22A; APP expression vector; processing and inhibition of processing control)

2a. 1 μg pMM321 and 1 μg pRBR186. Prior to transfection, 10 μM L-875,532 was added to cells (transfection solution for 3-5 were prepared in bulk)

3. and 3a. 1 μg pMM321 and 1 μg pcDNA3.1 zeo (+) APP(1-651)SW, K612V-(M1L)TATexonI

4. and 4a. 1 μg pMM321 and 1 μg pcDNA3.1 zeo (+) APP(1-651)SW, K612V-(M1L)TATexonI. Prior to transfection, 10 μM L-875,532 was added to the two wells.

5. and 5a. 1 μg pMM321 and 1 μg pcDNA3.1 zeo (+) APP(1-651)SW, K612V-(M1L)TATexonI. Prior to transfection, 10 μM Compound X was added to the two wells

6. 1 μg pMM321 and 1 μg pUCd5TAT (FIG. 24).

7. 1 μg pMM321 and 1 μg pUCd5 TAT. Prior to transfection, 10 μM L-875,532 was added to the cells.

Results:

Cells were assessed by eye under a fluorescence microscope the morning following transfection (˜20 hrs).

1 and 1a, 2 and 2a. Weak fluorescence

3 and 3a. Much stronger fluorescence

4 and 4a. Clear inhibition of fluorescence

5 and 5a. Possible inhibition of fluorescence, but doesn't look that great

6 and 7. Almost blindingly fluorescent.

At approximately 48 hours, cells were trypsinized, spun down, and resuspended in 100 μL PBS. The cellular contents of each well of the transfection plates were placed into one well of a 96-well fluorescence plate. Fluorescence was analyzed using the FLUOstar (485 excitation/538 emission). The results are shown in Table 3.

TABLE 3

| Transient transfections in H4 cells | Fluor Units |
|---|---|
| pMM321 | 4484 |
| pMM321 + L-875,532 | 3443 |
| pMM321 + pRBR186 | 2735 |
| pMM321 + pRBR186 + L-875,532 | 2161 |
| pMM321 + APP-TAT-ct32 | 20177 |
| pMM321 + APP-TAT-ct32 + L-875,532 | 8283 |
| pMM321 + APP-TAT-ct32 + Compound X | 11946 |
| pMM321 + pucd5-TAT | 61102 |

In Table 3, “APP-TAT-ct32” refers to pcDNA3.1 zeo (+) APP(1-651)SW, K612V-(M1L)TATexonI.

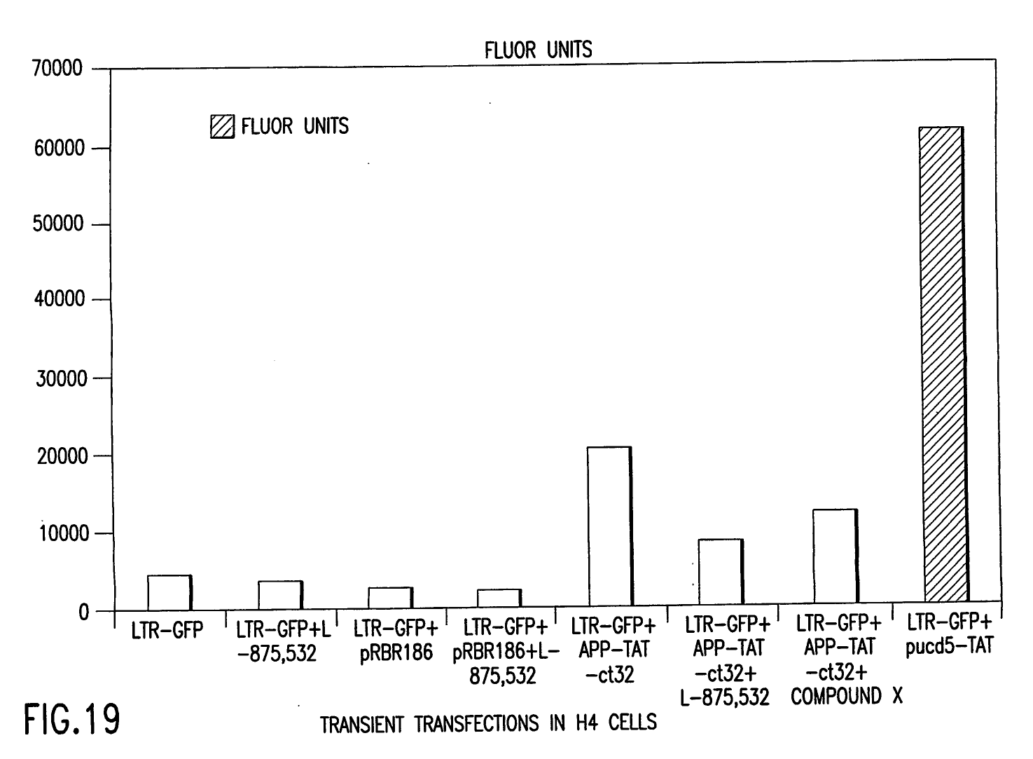

For a graphical presentation of these results, see FIG. 19. In FIG. 19, “LTR-GFP” refers to pMM321; “APP-TAT-ct32” refers to pcDNA3.1 zeo (+) APP(1-651)SW, K612V-(M1L)TATexonI. Compare the bar labeled “LTR-GFP+APP-TAT-ct32” with the bars labeled “LTR-GFP+APP-TAT-ct32+L-875,532” and “LTR-GFP+APP-TAT-ct32+Compound X.” Inhibition by both the β-secretase inhibitor (Compound X) and the γ-secretase inhibitor (L-875,532) is easily identified by the present invention.

### CONCLUSIONS

The data indicate that the expression of the fusion protein APP(1-651)SW, K612V-(M1L)TATexonI enhances transactivation through the LTR of pMM321 in a manner that depends on APP processing.

APP(1-651)SW, K612V-(M1L)TATexonI expressing cells were 6× brighter than pMM321 cells alone.

Treatment with L-875,532 decreased fluorescence 2.5×.

Treatment with Compound X decreased fluorescence 1.7×.

Expression of TAT via pucd5-TAT was almost blinding and was 19× above pMM321 alone, indicating that APP(1-651)SW, K612V-(M1L)TATexonI expression did not lead to levels of GFP as high as TAT alone. Despite the decreased activation shown by TAT when provided by the fusion protein, as compared with TAT driven by the AMLP (adenovirus major late promoter) in pucd5-TAT, the assay was easily able to identify both the β-secretase and the γ-secretase inhibitors.

Control plasmids (pMM321 and pMM321 +pRBR186) were dimly fluorescent and were not inhibited by L-875,532.

A lower level of inhibition by the β-secretase inhibitor is to be expected since the K612V mutation decreases alpha-secretase activity by 95% and thus some alpha-secretase cleavage is to be expected.

### EXAMPLE 4

### Construction of pcDNA3.1 (+) zeo APP(1-651)SW, K612V, GAL4-VP16(M1L) APP (664-695)

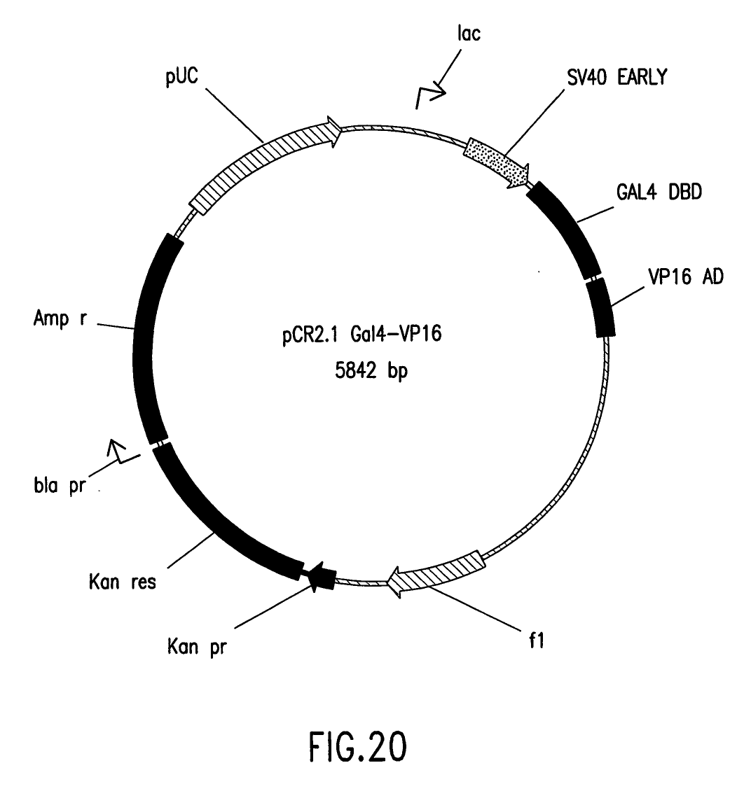

1. The GAL4-VP16 insert was prepared by PCR from pCR2.1 GAL4VP16 (FIG. 20) (Invitrogen, San Diego, Calif.). The PCR was performed to eliminate the N-terminal methionine by changing this methionine into a leucine.
40 ng pCR2.1 GAL4-VP16

0.2 μL GAL4-VP16 5′ oligo at 250 μM:

(SEQ ID NO:56)

5′-CTGAGATATCAAGCTACTGTCTTCTATCGAACAAGC-3′

EcoRV site underlined

0.2 μL GAL4VP16 3′ oligo (at 250 μM):

(SEQ ID NO:57)

5′-GCGCGATATCCCCACCGTACTCGTCAATTCC-3′

EcoRV site underlined
5 μL 10× Buffer
8 μL 25 mM MgCl₂
4 μL PCR dNTPs
0.25 μL AmpliTaq Gold
27.35 μL water
   Cycle:
Purified reactions using a Qiaquick column
Digested entire reaction using EcoRV
Ran the DNA on a 1% gel. Excised the band and purified using a QiaQuick gel purification kit

2. Digested pcDNA3.1 APP(1-651)/APP(664-695) with EcoRV and SAP treated. pcDNA3.1 APP(1-651)/APP(664-695) is an intermediate plasmid formed in the procedure described in Example 6. pcDNA3.1 APP(1-651)/APP(664-695) the first 651 amino acids of APP (with a Swedish version of the β-secretase cleavage site as well as the K612V mutation) fused in frame to the last 32 amino acids of APP.

3. Ligated pcDNA3.1 APP(1-651)/APP(664-695) -EcoRV digested to GAL4VP16 (EcoRV digested)

4. At this point, it was realized that the 3′ PCR primer for GAL4-VP16 put the APP(664-695) fragment out of frame. The APP(664-695) fragment was then re-PCR'd using the following protocol:
1 μL pcDNA3.1 APP(1-651)-Gal4VP16-APP(664-695)
50 nM APP NotI 5′ ct32 in frame with GAL4-VP16

50 nM APP Noti 3′ ct32

(SEQ ID NO:58)

5′(p)CTGCTGTGGCGGCCGCCTAGTTCTGCATCTGCTC)

NotI site underlined
1 μL PCR dNTPs (10 mM each dNTP, Roche)
5 μL 10× Expand Buffer with MgCl₂
40 μL water
1 μL Expand polymerase (Roche)

The PCR fragment was run on a 4% agarose gel and gel-purified using a QiaQuick gel purification column

The fragment was digested with NotI and purified using a QiaQuick PCR purification column.

5. APP(1-651)-Gal4VP16-APP(664-695) was re-miniprepped. Miniprep #1 was digested with NotI, run on a 1% gel, the upper band was then isolated and SAP-treated.

6. APP(1-651)-Gal4VP16/NotI digested/SAP-treated was ligated to APP(664-695).

7. Minipreps containing inserts were sequenced to verify the orientation of the insert.

### EXAMPLE 5

### Construction of pcDNA3.1 zeo (+) APP(1-651)wt, K612V-TATexonI(M1L) APP(664-695)

This procedure replaced a fragment of APP in pcDNA3.1 zeo (+) APP(1-651)SW, K612V-TATexonI(N41L) APP (664-695) that contained the Swedish mutation with a corresponding fragment from pRBR121 containing the wild-type β-secretase cleavage site rather than the Swedish β-secretase cleavage site.

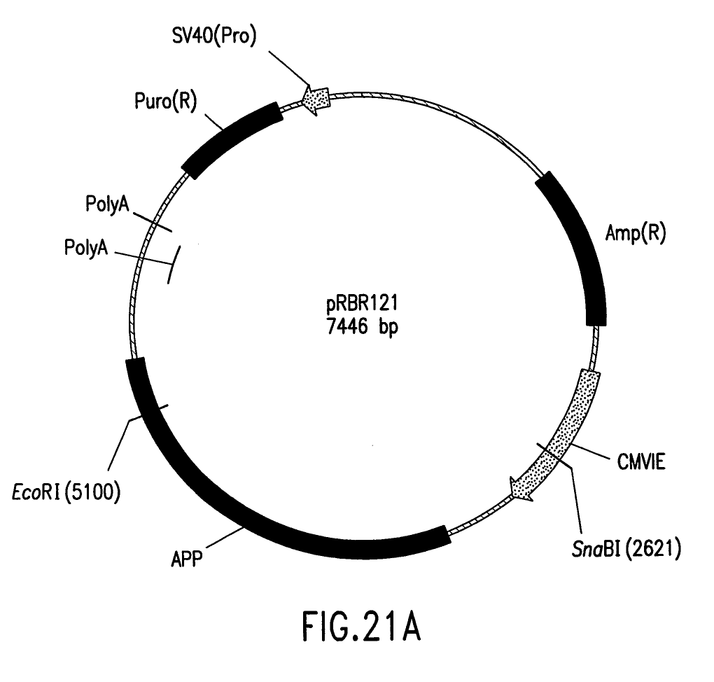

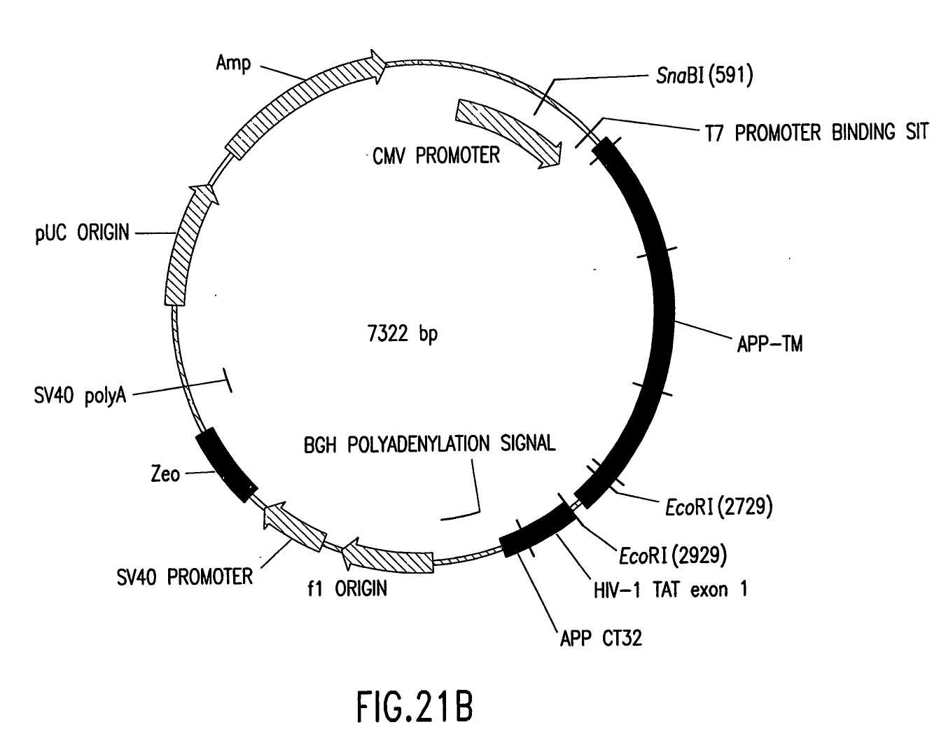

1. pcDNA3.1 zeo (+) APP(1-651)SW, K612V-TATexonI(M1L) APP (664-695) (FIG. 21B) was digested with SnaBI and then EcoRI

17 μL miniprep DNA

2 μL 10× buffer

1 μL SnaBI (NEB)

Digest was purified using Qiaquick PCR purification kit. Entire digest was then cleaved with EcoRI for 2 hours.

2. pRBR121 (FIG. 21A) was digested with SnaBI and then EcoRI

5 μg pRBR121

5 μL 10× buffer

2.5 μL SnaBI (NEB)

q.s. 50 μL with water

The digest was purified using a Qiaquick PCR purification kit. The entire digest was then cleaved with EcoRI for 2 hours.

3. Both digests were run out on a 1% agarose gel. From the pRBR121 lane, the 2.4 kb SnaBI-EcoRI fragment containing the wild-type β-secretase cleavage site was isolated.

From pcDNA3.1 zeo (+) APP(1-651)SW, K612V-TATexonI(MIL) APP (664-695) digest, BOTH the 5 kb SnaBI-EcoRI backbone fragment AND the 200 bp EcoRI-EcoRI fragment were isolated (see FIG. 21B).

4. A three-part ligation using equal molar ratios of the three fragments was carried out:

The assumption was made that, since the starting plasmids were of similar sizes and the same amount was digested for each plasmid, the recovered fragments would be in approximately equal molar ratios.

Vector alone:

1μL APP-TAT-ct32 SnaBI/EcoRI 5Kb fragment
7 μL water
2 μL 5× buffer
10 μL 2× buffer (Roche rapid ligation kit)
1 μL T4 ligase
   1+1+1
1 μL pcDNA3.1 zeo (+) APP(1-651)SW, K612V-TATexonI(M1L) APP (664-695) SnaBI/EcoRI 5KB fragment
1 μL pRBR121 SnaBI/EcoRI 2.4 Kb insert
1 μL pcDNA3.1 zeo (+) APP(1-651)SW, K612V-TATexonI(MIL) APP (664-695) EcoRI/EcoRI 200 bp insert
5 μL water
2 μL 5× buffer
10μL 2× buffer
1 μL T4 ligase
   1+1+ . . . 1 (in this 3-pt ligation, the ligation of two of the fragments together was done 1^{st}, then the third fragment was added)
1 μL pcDNA3.1 zeo (+) APP(1-651)SW, K612V-TATexonI(M1L) APP (664-695) SnaBI/EcoRI 5Kb backbone
1 μL pcDNA3.1 zeo (+) APP(1-651)SW, K612V-TATexonI(M1L) APP (664-695) EcoRI/EcoRI 200 bp insert
5 μL water
2 μL 5× buffer
10 μL 2× buffer
1 μL T4 ligase
waited 5 minutes
then added 1 μL pRBR121 SnaBI/EcoRI 2.4 kb insert
   1+1+3
1 μL pcDNA3.1 zeo (+) APP(1-651)SW, K612V-TATexonI(M1L) APP (664-695) SnaBI/EcoRI 5 kb backbone
1 μL pRBR121 SnaBI/EcoRI 2.4 kb insert
3 μL pcDNA3.1 zeo (+) APP(1-651)SW, K612V-TATexonI(MIL) APP (664-695) EcoRI/EcoRI 200 bp insert
3 μL water
2 μL 5× buffer
10 μL 2× buffer
1 μL T4 ligase

Transformed and plated out 200 μL. The number of colonies in the vector+insert ligations far exceeded the number of colonies in the vector alone ligation. Picked 12 colonies from 1+1+ . . . 1.
Picked 6 colonies from 1+3.
Miniprepped
Digested with EcoRI to ensure that small 200 bp fragment was incorporated.
   Results:

Minipreps #10 and 15 contained 200 bp EcoRI fragment.

Oriented with Bam HI digestion.

Sequenced with sAPPb F2 and F3 primers. Miniprep #15 contains both inserts in the correct orientation.

### EXAMPLE 6

### Construction of pcDNA3.1 zeo (+) APP(1-651)SW, K612V-TATexonI(M1L) APP(664-695)

1. PCR of APP (664-695):
The starting material was the pRBR186 plasmid (FIG. 22A).
   PCR of APP (664-695)
1 mg pRBR186

50 nM 5′ oligo

(SEQ ID NO:59)

(5′-TGCCCCGCGCGGCCGCGCGATGCTGCCCGG-3′)

NotI site underlined

50 nM 3′ oligo

(SEQ ID NO:60)

(5′-(p)ATGGTGTGGCGGCCGCAGACGCCGCTGTCACC-3′)

NotI site underlined
1 μL Roche PCR nucleotides
5 μL 10× Expand buffer
40 μL water
1 μL Expand

Cycle: (94° C., 5 min)−25×(94° C., 30 sec; 42° C., 1 min; 72° C., 2 min)−72° C. ×6 min−4° C. hold.

The ˜100 bp fragment was gel purified (4% agarose, 1×TBE gel)

The gel-purified fragment was ligated into NotI digested, Shrimp Alkaline Phosphatase-treated pcDNA3.1 zeo (+) (Invitrogen). The presence of the insert and its orientation was confirmed by sequencing.

2. PCR of APP(1-651):

1 ng pRBR186

50 nM 5′ oligo

(SEQ ID NO:61)

(5′-(p)AGCGCACAAGCTTCCCCGCGCAGGGTCGCGATGCTG-3′)

HindIII site underlined, Met(1) ATG of APP in bold

50 nM 3′ oligo

(SEQ ID NO:62)

(5′-(p)GGATGTAAGCTTTTTCTTCTTCAGCATCACCAAGG-3′)

HindIII site underlined
1 μL Roche PCR nucleotides
5 μL 10× Expand buffer
40 μL water
1 μL Expand

Cycle: (94° C., 5 min)−25×(94° C., 30 sec; 37° C., 1 min; 72° C. 2.5 min)−72° C.×6 min−4° C. hold

The amplified fragment was isolated on an agarose gel. The fragment was purified from the gel using Qiaquick Gel purification columns. The fragment was digested with HindIII. The amount of the fragment was too small to subclone, so the PCR was repeated using 1 μL of the amplified fragment and carrying out 5 reactions simultaneously.

The fragments were purified from these reactions using a QiaQuick PCR purification kit. The fragments were eluted in 30 μL and digested with HindIII for 2 hours. The digested fragments were then gel purified.

The purified fragments were ligated to pcDNA3.1 zeo (+) APP(664-695) that had been digested with HindIII and SAP treated. This gave the intermediate plasmid pcDNA3.1 zeo (+) APP(1-651)/APP(664-695).

3. PCR of (M1L) TAT:

The starting material was NL4-3 viral plasmid (FIG. 22B).

PCR reaction:

1 ng NL4-3 viral plasmid

50 nM TAT 5′ RV Met-Leu PCR primer

(SEQ ID NO:63)

(5′-TGCAGATATCCTGGAGCCAGTAGATCCTAGAC-3′)

EcoRV site underlined, Met-Leu mutation in bold

50 nM TAT 3′ RV Met-Leu PCR primer

(SEQ ID NO:64)

(5′-GCTGGATATCCTCTGCTTTGATAGAGAAGC-3′)

EcoRV site underlined
1 μL PCR dNTPs
5 μL PCR 10× buffer with MgCl₂
40 μL water
1 μL Expand polymerase
   Cycle:
94° C. for 5 min
[30 sec 94° C., 1 min 42° C., 1 min 72° C.]×25 cycles
5 min at 72C
hold at 4° C.

The insert was purified over QiaQuick PCR purification column

The entire reaction was digested with 30 units EcoRV for 3 hours

The ˜200 bp insert was gel purified.

pcDNA3.1 zeo (+) APP(1-651)/APP(664-695) was digested with EcoRV, and then SAP treated

The Met1-Leu TAT fragment was ligated to pcDNA3.1 zeo (+) APP(1-651)/APP(664-695).

A map of the resulting plasmid is shown in FIG. 22C.

### EXAMPLE 7

### Design of Novel Expression Vector for Expression of APP(1-651)SW, K612V-TATexonI(M1L) APP(664-695)

Purpose:

To provide a low level expression of APP(1-651)SW, K612V-TATexonI(M1L) APP(664-695), a prokaryotic selectable marker that is NOT ampicillin (read-through of the β-lactamase gene is sometimes a problem), and a eukaryotic selectable marker that is NOT zeocin (zeo is the marker for the reporter plasmids in some embodiments).

Methods

1. The dEYFP gene was removed from pd2EYFP (Clontech, Palo Alto, Calif.) using BamHI and NotI. The 5′ overhangs was filled in using Klenow, and the plasmid was re-circularized.
pd2EYFP plasmid was digested with BamHI, NotI.
Ran reaction on 1% agarose gel. Digestion was complete. Cut out 3.4 kb band.
Purified using Qiagen Gel Extraction Kit.
   Klenow Fill-In:
˜4 μg plasmid backbone
7.5 μL NEB buffer 2
33 μM each dNTP (diluted from Roche PCR dNTPs)
water to 75 μL
4 μL Roche Klenow fragment (4 units)
Incubated at room temperature for 15 minutes
Heat inactivated at 70° C.
Took 1 μL fill-in reaction.
Diluted to 8 μL with water
Added 2 μL 5×DNA buffer
Added 10 μL 2× Ligation Buffer
Added 1 μL T4 DNA ligase
Incubated at room temperature

Transformed 2 μL ligation into Invitrogen maximum efficiency DH5alpha competent cells.

Plated out on Kanamycin plates. Lots of colonies.

2. The RSV promoter from pREP4 (Invitrogen) was excised using BglII and HindIII and cloned into the re-circularized plasmid.
Digested 5 μg pREP4 with HindIII
Purified using Qiagen PCR purification kit
Digested with BglII.

The RSV promoter fragment was gel purified and cloned into the re-circularized plasmid.

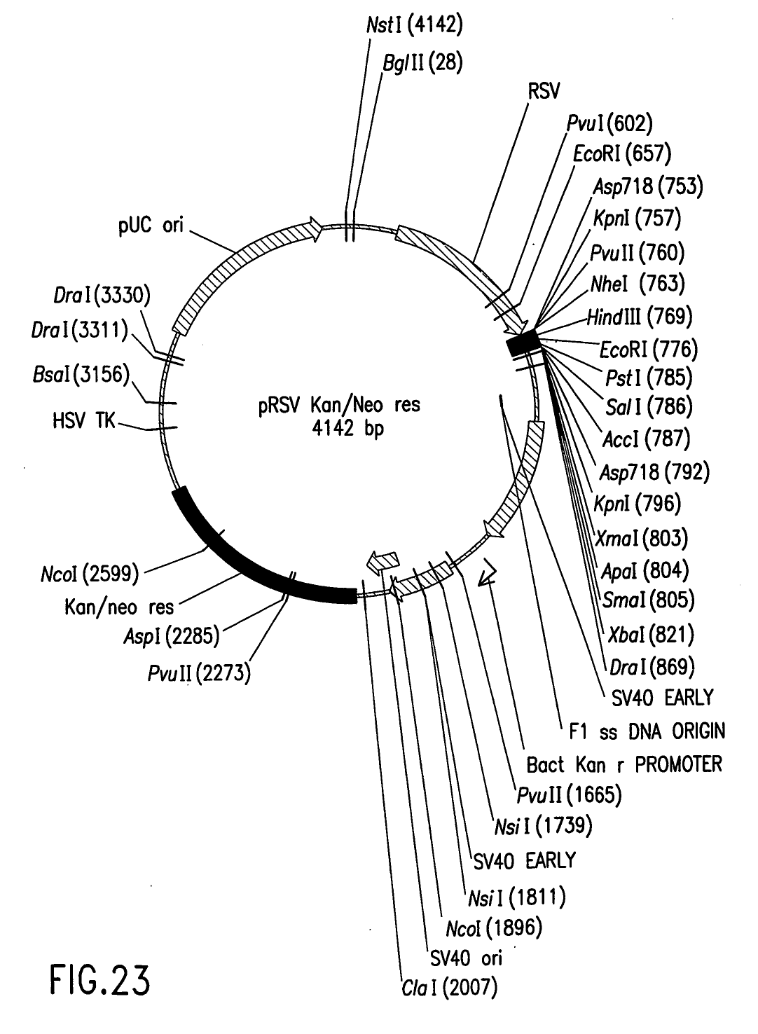

3. The resulting expression vector (pRSV Kan/Neo res; FIG. 23) has the eukaryotic RSV promoter 5′ to the pd2EYFP polylinker, SV40 driving neo and kanamycin prokaryotic selection, and a pUC ori for high levels of replication in bacteria.

### EXAMPLE 8

### Use of APP(1-651)SW, K612V-TATexonI(M1L) APP(664-695) in HeLa Cells With a β-galactosidase Reporter Gene

The following demonstrates the practice of the present invention with the APP(1-651)SW, K612V-TATexonI(M1L) APP(664-695) fusion protein (SEQ ID NO:2) and β-galactosidase as a reporter gene. P4-R5 cells are HeLa cells that contain a stably integrated β-galactosidase reporter gene under the control of the HIV1 LTR.

Materials:

1.) Cells: P4-R5 cells

2.) DNA: 0.78 μg/μL pcDNA3.1 zeo (+)APP(1-651)SW, K612V-TATexonI(M1L) APP(664-695)

3.) Transfection reagents: FUGENE®

4.) Media: OPTIMEM®
cDMEM (−)phenol red/10% FBS

5.) Compounds: Compound X (β-secretase inhibitor) 10 mM
L-875,532 (γ-secretase inhibitor) 10 mM
   Protocol:
   Day 1

1.) Cell count on P4-R5 cells=7.6×10⁵ cells per ml in cDMEM (−)PR. Seeded sterile white luminometer TC plates with the following cell numbers:

### 10 ml

5×10³/well=0.75 ml in 9.25 ml media
1.0×10⁴/well=1.5 ml in 8.5 ml media
Seeded 100 μL cells per well.
Incubate overnight at 37° C., 5% CO₂.
   Day 2

2.) Made up media with appropriate dilutions of inhibitors.
On no-inhibitor controls, added 100 μL of cDMEM with 1% DMSO
On wells with Compound X, added 10 μM inhibitor in cDMEM
On wells with L-875,532, added 10 μM inhibitor in cDMEM

3.) Prior to transfection, pulled off media on P4-R5 cells and replaced with media −/+inhibitor.
FUGENE® transfection:
For FUGENE® transfection:

4.) Added 600 μL of OPTIMEM® to sterile EPPENDORF® tube and carefully added 30 μL FUGENE® to media, without touching walls of tube. Incubated at room temperature for 5 minutes.
In separate EPPENDORF® tubes, added each DNA.
Added FUGENE®/OPTIMEM® dropwise to DNA; incubated at room temperature for 15 minutes.

Added 15 μL/well of DNA/FUGENE®/OPTIMEM® dropwise to media in appropriate wells on P4-R5 cells, swirling to mix.

TABLE 4

| Transfection | | | | |
|---|---|---|---|---|
| number | Conc of DNA | Vol of DNA | Vol of FUGENE ® | Vol of sterile OPTIMEM ® |
| APP-ML-Tat-APPct | 0.78 μg/μL | 5 μg = 6.5 μL | 30 μL of FUGENE ® | 600 μL of OPTIMEM ® |
| pUCd5TAT | 1.24 μg/μL | 5 μg = 4.0 μL | 30 μL of FUGENE ® | 600 μL of OPTIMEM ® |
| p243-4 | 0.56 μg/μL | 5 μg = 9 μL | 30 μL of FUGENE ® | 600 μL of OPTIMEM ® |

In Table 4, “APP-ML-Tat-APPct” refers to pcDNA3.1 zeo (+)APP(1-651)SW, K612V-TATexonI(M1L) APP(664-695). “pUCd5TAT” is an expression vector that serves as a positive control for TAT expression, since it is a construct in which TAT is under the control of a strong, constitutive promoter (see FIG. 24). “p243-4” is a control expression vector that directs the expression of APP.

5.) Plates were transferred to an incubator and incubated for 48 hours to allow expression and processing of the proteins.

Day 4

6.) The protocol below was followed for lysis of the cells and measurement of β-galactosidase in the cell lysates.

Measurement of β-galactosidase in lysates of transfected cells.

1. Removed TROPIX® chemiluminescence kit(s) from cold room, allowed to come to room temperature in a 37° C. water bath.

2. β-galactosidase standards were prepared: Made 1:5000 dilution of β-galactosidase stock (1 mg/ml) in lysis buffer. Did 2 fold dilutions.

3. Diluted TROPIX® substrate 1:25 into buffer. (Made enough for 100 μL/well).

4. Added to reservoir and added 100 μL/well.

5. Added 10 μL of β-galactosidase standards to column 12 on plate and incubated in dark for 1 hour.

6. Read immediately in luminometer using standard file. Filled in required fields, read plate.

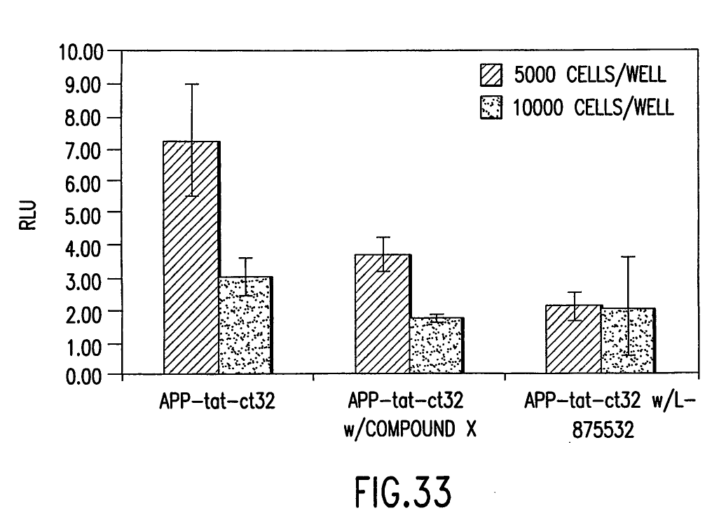

The results are shown in FIG. 33. FIG. 33 demonstrates that the present invention was able to identify both the β-secretase inhibitor (Compound X) and the γ-secretase inhibitor (L-875,532). In FIG. 33, “APP-tat-ct32” refers to pcDNA3.1 zeo (+)APP(1-651)SW, K612V-TATexonI(M1L) APP(664-695). Although not indicated in FIG. 33, the results for the controls were as expected: a large transactivation of the LTR by pUCd5TAT was observed which was not affected by either inhibitor. No transactivation was seen with p243-4.

### EXAMPLE 9

### Comparison of the Use of APP(1-651)SW, K612V-TATexonI(M1L) APP(664-695) and APP(1-651)wt, K612V-TATexonI(M1L) APP(664-695) With a β-galactosidase Reporter Gene

The following shows a side-by-side comparison of the practice of the present invention with the APP(1-651)SW, K612V-TATexonI(M1L) APP(664-695) fusion protein (SEQ ID NO:2) and the APP(1-651)wt, K612V-TATexonI(M1L) APP(664-695) fusion protein (SEQ ID NO:4). P4-R5 cells are HeLa cells that contain a stably integrated β-galactosidase reporter gene under the control of the HIV1 LTR.

Materials:

1.) Cells: P4-R5 cells

2.) DNA: 0.78 μg/μL pcDNA3.1 neo (+) APP(1-651)SW, K612V-TATexonI(M1L) APP(664-695)
0.812 μg/μL pcDNA3.1 neo (+) APP(1-651)wt, K612V-TATexonI(M1L) APP(664-695)
1.24 μg/μL pUCd5TAT
0.56 μg/μL p243-4

3.) Transfection reagents: FUGENE®

4.) Media: OPTIMEM®
cDMEM (−)phenol red/10% FBS

5.) Compounds: Compound X (β-secretase inhibitor) 10 mM
L-875,532 (γ-secretase inhibitor) 10 mM
   Day 1

1.) Cell count on P4-R5 cells=5×10⁵ cells per ml in cDMEM (−)PR. Seeded sterile white luminometer TC plates with the following cell numbers:

### 10 ml

5×10³/well=4.0 ml in 36.0 ml media
Diluted 1:1 into media and seeded one plate at 2.5×10³/well.
Seeded 100 μL cells per well.
Incubated overnight at 37° C., 5% CO₂.
   Day 2

2.) Made up media with appropriate dilutions of inhibitors.
On no-inhibitor controls, added 100 μL of cDMEM with 1% DMSO
On wells with Compound X, added titration curve from 100 μM inhibitor in cDMEM (−)PR.
On wells with L-875,532, added titration curve from 100 μM inhibitor in cDMEM (−)PR.

3.) Prior to transfection, pulled off media on P4-R5 cells and replaced with media −/+ inhibitor.

FUGENE® Transfection:

4.) Added volume of OPTIMEM® to sterile EPPENDORF® tube and carefully added correct volume of FUGENE® to media, without touching walls of tube. Incubated at room temperature for 5 minutes.
In separate EPPENDORF® tubes, added each DNA, as outlined below.
Added FUGENE®/OPTIMEM® dropwise to DNA; incubated at room temperature for 15 minutes.

Added 15 μL/well of DNA/FUGENE®/OPTIMEM® dropwise to media in appropriate wells on P4-R5 cells, swirling to mix.

TABLE 5

| Transfection number | Conc of DNA | Vol of DNA | Vol of FUGENE ® | Vol of sterile OPTIMEM ® |
|---|---|---|---|---|
| 1.) APP-ML-Tat-APPct (Sw) | 0.78 μg/μL | 10 μg = 13 μL | 60 μL of FUGENE ® | 1200 μL of OPTIMEM ® |
| 2.) APP-ML-Tat-APPct (WT) | 0.812 μg/μL | 10 μg = 12.2 μL | 60 μL of FUGENE ® | 1200 μL of OPTIMEM ® |
| 3.) pUCd5TAT | 1.24 μg/μL | 5 μg = 4.0 μL | 30 μL of FUGENE ® | 600 μL of OPTIMEM ® |
| 4.) p243-4 | 0.56 μg/μL | 5 μg = 9 μL | 30 μL of FUGENE ® | 600 μL of OPTIMEM ® |

In Table 5, “APP-ML-Tat-APPct (Sw)” refers to pcDNA3.1 neo (+) APP(1-651)SW, K612V-TATexonI(M1L) APP(664-695). “APP-ML-Tat-APPct (WT)” refers to pcDNA3.1 neo (+) APP(1-651)wt, K612V-TATexonI(M1L) APP(664-695). “pUCd5TAT” is an expression vector that serves as a positive control for TAT expression, since it is a construct in which TAT is under the control of a strong, constitutive promoter (see FIG. 24). “p243-4” is a control expression vector that directs the expression of APP.

5.) Plates were transferred to an incubator and incubated for 36 hours to allow expression and processing of proteins.

Day 4

6.) The protocol below was followed for lysis of the cells and measurement of β-galactosidase in the cell lysates.

Measurement of β-galactosidase in Lysates of Transfected Cells.

1. Removed TROPIX® chemiluminescence kit(s) from cold room, allowed to come to room temperature in a 37° C. water bath.

2. β-galactosidase standards were prepared: Made 1:5000 dilution of β-galactosidase stock (1 mg/ml) in lysis buffer. Did 2 fold dilutions.

3. Diluted TROPIX® substrate 1:25 into buffer. (Made enough for 120 μL/well).

4. Added to reservoir and added 120 μL/well.

5. Added 10 μL of β-galactosidase standards to column 12 on plate and incubated in dark for 1 hour.

6. Read immediately in luminometer using standard file. Filled in required fields, read plate.

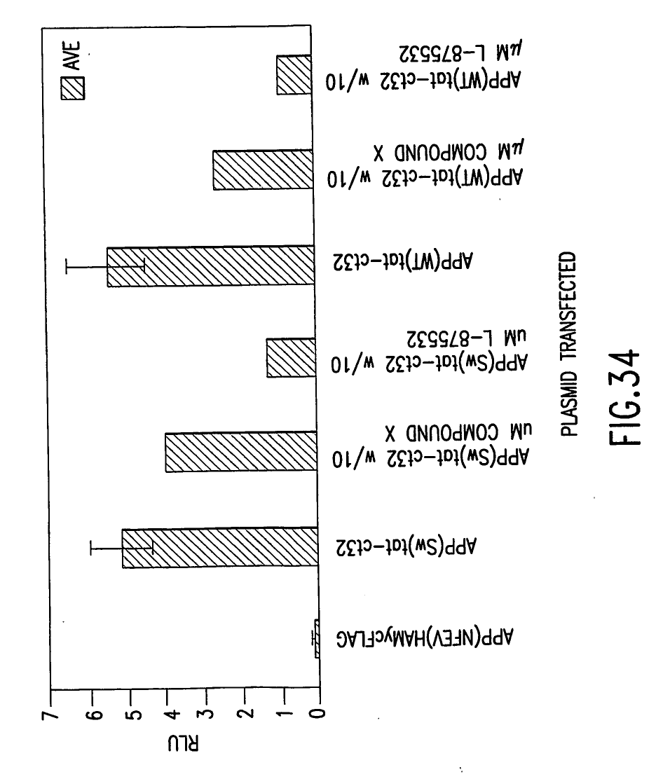

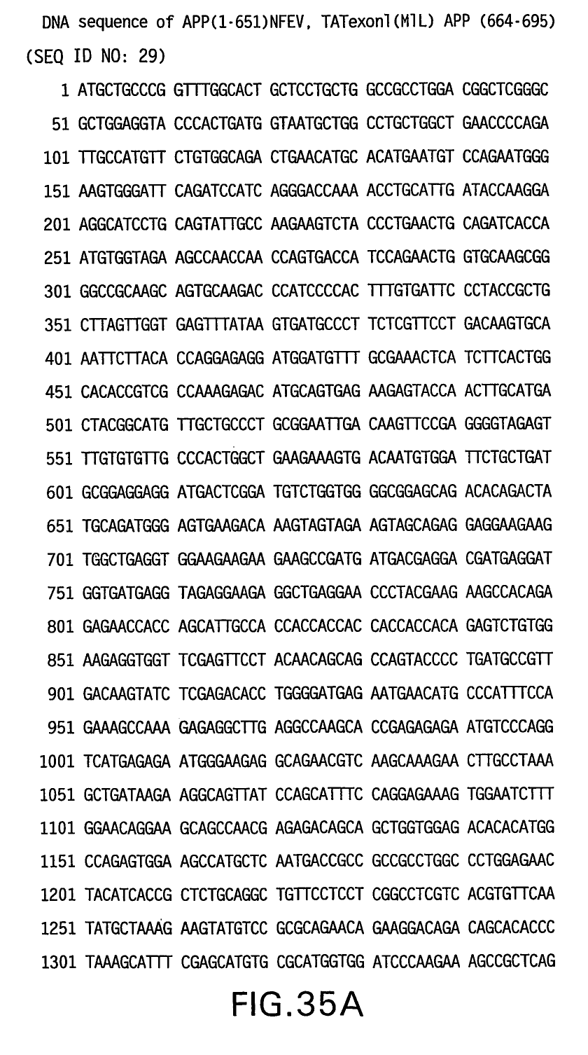

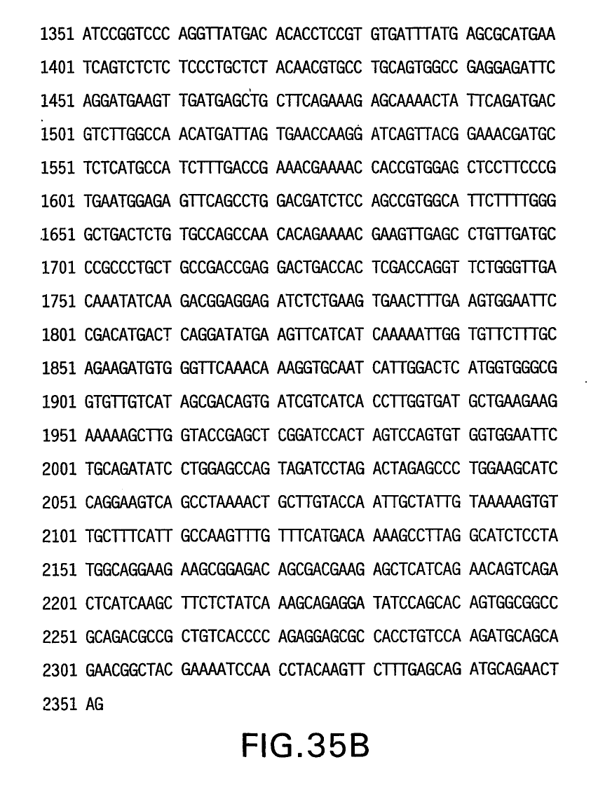

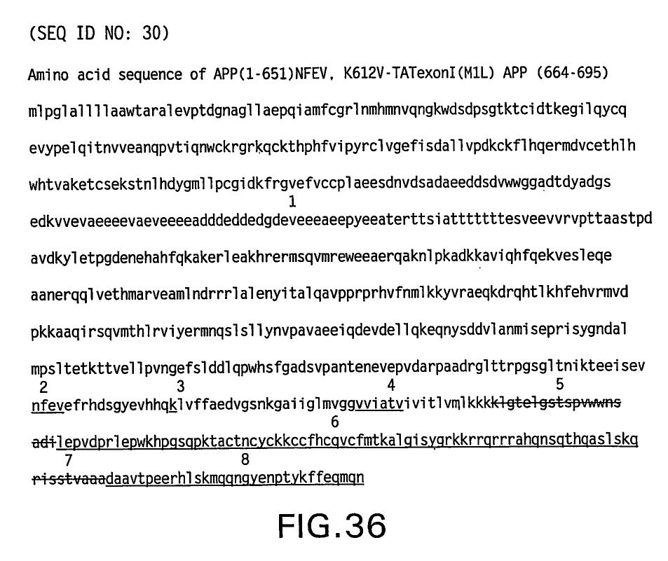

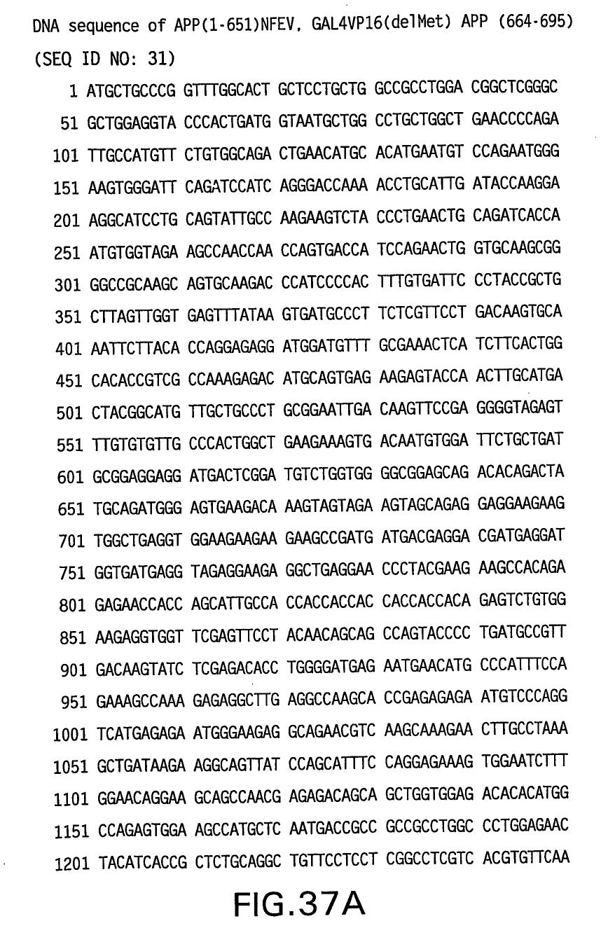

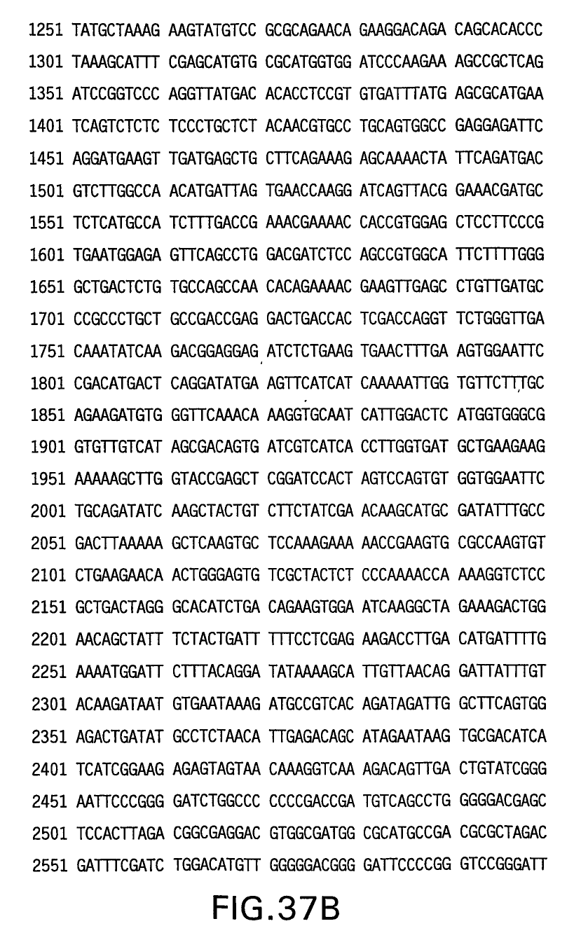

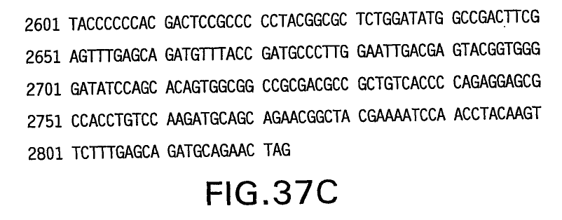

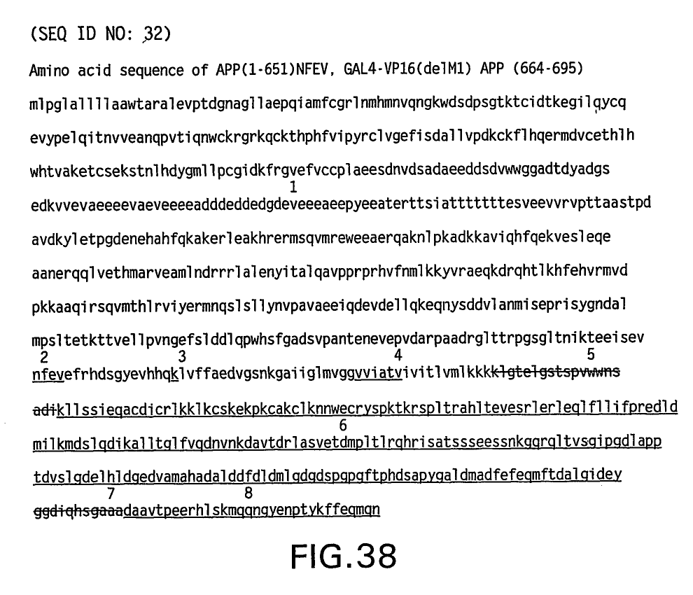

The results are shown in FIG. 34. In FIG. 34, “APP(NFEV)HAMycFLAG” refers to a protein that is a variant of APP in which NFEV is present at the β-secretase cleavage site and there are epitope tags in the amino terminal portion of the protein but there is no transcription factor fused to APP. “APP(Sw)tat-ct32” refers to pcDNA3.1 neo (+) APP(1-651)SW, K612V-TATexonI(M1L) APP(664-695). “APP(WT)tat-ct32” refers to pcDNA3.1 neo (+) APP(1-651)wt, K612V-TATexonI(M1L) APP(664-695). FIG. 34 shows that the Swedish version and the wild-type version of APP appear to work about equally well in the assay.

### EXAMPLE 10

### L-685,458

L-685,458 is a γ-secretase inhibitor having the following structure:

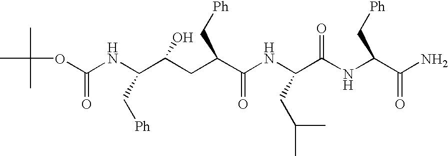

L-685,458 contains an hydroxyethylene dipeptide isostere and is thought to function as a transition state analog mimic of aspartyl proteases (Shearman et al., 2000, Biochemistry 39:8698-8704). L-685,458 was prepared as follows: {1S-Benzyl-4R-[1-(1S-carbamoyl-2-phenylethylcarbamoyl)-1S-3-methylbutylcarbamoyl]-2R-hydroxy-5-phenylpentyl}carbamic acid tert-butyl ester (L-685,458) was prepared by the coupling of 2R-benzyl-5S-tert-butoxycarbonylamino-4R-(tert-butyldimethylsilanyloxy)-6-phenylhexanoic acid (Evans et al., 1985, J. Org. Chem. 50:4615-4625) with Leu-Phe-NH2 followed by deprotection with tetrabutylammonium fluoride. The synthesis of {1S-benzyl-4R-[1-(1S-carbamoyl-2-phenylethylcarbamoyl)-1S-3-methylbutylcarbamoyl]-2S-hydroxy-5-phenylpentyl}carbamic acid tert-butyl ester (L-682,679) has been described previously (De Solms et al., 1991, J. Med. Chem. 34:2852-2857). {1S-Benzyl-4R-[1-(1S-carbamoyl-2-phenylethylcarbamoyl)-1S-3-methylbutylcarbamoyl]-2-oxo-5-phenylpentyl}carbamic acid tert-butyl ester (L-684,414) was prepared by pyridinium dichromate-mediated oxidation of L-682,679.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of which are incorporated by reference in their entireties.

What is claimed is:

1. A DNA molecule comprising a nucleotide sequence encoding a fusion protein comprising amino acids 589-651 selected from the group consisting of wild type APP₆₉₅, the Swedish version of APP₆₉₅ and the NFEV (SEQ ID NO:40) version of APP₆₉₅ and a transcription factor where the transcription factor is fused in frame to the carboxyl terminus of amino acids 589-651.

2. The DNA molecule of claim 1 where amino acids 589-651 contain a K612V mutation.

3. The DNA molecule of claim 1 where the nucleotide sequence further encodes amino acids 664-695 of APP₆₉₅ wherein amino acids 664-695 are fused in frame to the carboxyl terminus of the transcription factor.

4. The DNA molecule of claim 1 where the transcription factor is selected from the group consisting of: HIV-1 TAT, Gal4-VP16, the entire Gal4 protein, LexA-VP16, E12, E47, Twist, Papillomavirus E2, EBV Zta, BIV TAT, HIV-2 TAT, or SIV TAT.

5. The DNA molecule of claim 3 where the fusion protein is selected from the group consisting of: APP(1-651)wt, K612V-TATexonI(M1L) APP (664-695) (SEQ ID NO:4); APP(1-651)wt, K612V, Gal4-VP16(M1L) APP (664-695) (SEQ ID NO:8); APP(1-651)wt, TATexonI(M1L) APP (664-695) (SEQ ID NO:12); and APP(1-651)wt, Gal4-VP16(M1L) APP (664-695) (SEQ ID NO:16).

6. The DNA molecule of claim 3 where the fusion protein is selected from the group consisting of: APP(1-651)SW, K612V-TATexonI(M1L) APP (664-695) (SEQ ID NO:2); APP(1-651)SW, K612V, Gal4-VP16(M1L) APP (664-695) (SEQ ID NO:6); APP(1-651)SW, TATexonI(M1L) APP (664-695) (SEQ ID NO:10); and APP(1-651)SW, Gal4-VP16(M1L) APP (664-695) (SEQ ID NO:14).

7. The DNA molecule of claim 3 where the fusion protein is selected from the group consisting of: APP(1-651)NFEV, K612V-TATexonI(M1L) APP (664-695) (SEQ ID NO:23) and APP(1-651)NFEV, K612V, GAL4-VP16(M1L) APP (664-695) (SEQ ID NO:25).

8. An expression vector comprising the DNA molecule of claim 1.

9. A eukaryotic cell comprising the DNA molecule of claim 1.

10. The cell of claim 9 further comprising a reporter gene where the reporter gene is under the control of a regulatory DNA sequence that is capable of being activated by the transcription factor.

11. A method of identifying a substance that inhibits APP processing comprising:
(a) providing a recombinant eukaryotic cell which:
(i) expresses a fusion protein comprising amino acids 589-651 selected from the group consisting of wild type APP₆₉₅, the Swedish version of APP₆₉₅ and the NFEV (SEQ ID NO:40) version of APP₆₉₅ and a transcription factor where the transcription factor is fused in frame to the carboxyl terminus of amino acids 589-651; and
(ii) comprises a reporter gene operably linked to a regulatory DNA sequence which capable of being activated by the transcription factor;
(b) measuring the level of reporter gene product in the cell in the absence of the substance;
(c) adding the compound to the cell and measuring the level of reporter gene product in the cell in the presence of the substance;
where a decrease in the level of reporter gene product in the presence as compared to the absence of the substance indicates that the substance inhibits APP processing.

12. The method of claim 11 where amino acids 589-651 contain a K612V mutation.

13. The method of claim 11 where the transcription factor is selected from the group consisting of: HIV-1 TAT, Gal4-VP16, the entire Gal4 protein, LexA-VP16, E12, E47, Twist, Papillomavirus E2, EBV Zta, BIV TAT, HIV-2 TAT, or SIV TAT.

14. The method of claim 11 where the fusion protein is selected from the group consisting of: APP(1-651)wt, K612V-TATexonI(M1L) APP (664-695) (SEQ ID NO:4); APP(1-651)wt, K612V, Gal4-VP16(M1L) APP (664-695) (SEQ ID NO:8); APP(1-651)wt, TATexonI(M1L) APP (664-695) (SEQ ID NO:12); and APP(1-651)wt, Gal4-VP16(M1L) APP (664-695) (SEQ ID NO:16).

15. The method of claim 11 where the fusion protein is selected from the group consisting of: APP(1-651)SW, K612V-TATexonI(M1L) APP (664-695) (SEQ ID NO:2); APP(1-651)SW, K612V, Gal4-VP16(M1L) APP (664-695) (SEQ ID NO:6); APP(1-651)SW, TATexonI(M1L) APP (664-695) (SEQ ID NO:10); and APP(1-651)SW, Gal4-VP16(M1L) APP (664-695) (SEQ ID NO:14).

16. The method of claim 11 where the fusion protein is selected from the group consisting of: APP(1-651)NFEV, K612V-TATexonI(M1L) APP (664-695) (SEQ ID NO:23) and APP(1-651)NFEV, K612V, Gal4-VP16(M1L) APP (664-695) (SEQ ID NO:25).

17. A method of identifying a substance that inhibits APP processing comprising:
(a) providing a recombinant eukaryotic cell which:
(i) expresses a fusion protein comprising an amino acid sequence from APP that is capable of being cleaved by both β-secretase and γ-secretase and a transcription factor where the transcription factor is fused in frame to the amino acid sequence from APP; and
(ii) comprises a reporter gene operably linked to a regulatory DNA sequence which capable of being activated by the transcription factor;
(b) measuring the level of reporter gene product in the cell in the absence of the substance;
(c) adding the compound to the cell and measuring the level of reporter gene product in the cell in the presence of the substance;
where a decrease in the level of reporter gene product in the presence as compared to the absence of the substance indicates that the substance inhibits APP processing.

18. The method of claim 17 where the amino acid sequence from APP comprises an amino acid sequence selected from the group consisting of 589-651 of APP₆₉₅, 589-651 of the Swedish version of APP₆₉₅, and 589-651 of the NFEV version of APP₆₉₅.

19. The method of claim 17 where the amino acid sequence from APP contains the amino acid sequence NLDA (SEQ ID NO:38) at the β-secretase cleavage site instead of the wild-type sequence KMDA (SEQ ID NO:34).

20. The method of claim 17 where the amino acid sequence from APP contains the amino acid sequence NFEV (SEQ ID NO:40) at the β-secretase cleavage site instead of the wild-type sequence KMDA (SEQ ID NO:34).


SEQUENCE LISTING (64 sequences)

SEQ ID NO: 1 - DNA, 2352 bp, human
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttataagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaatctagatgcagaattccgacatgactcaggatatgaagttcatcatcaagtattggtgttctttgcagaagatgtgggttcaaacaaaggtgcaatcattggactcatggtgggcggtgttgtcatagcgacagtgatcgtcatcaccttggtgatgctgaagaagaaaaagcttggtaccgagctcggatccactagtccagtgtggtggaattctgcagatatcctggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagaggatatccagcacagtggcggccgcagacgccgctgtcaccccagaggagcgccacctgtccaagatgcagcagaacggctacgaaaatccaacctacaagttctttgagcagatgcagaactag

SEQ ID NO: 2 - protein, 783 aa, fusion protein - human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFISDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVNLDAEFRHDSGYEVHHQVLVFFAEDVGSNKGAIIGLMVGGVVIATVIVITLVMLKKKKLGTELGSTSPVWWNSADILEPVDPRLEPWKHPGSQPKTACTNCYCKKCCFHCQVCFMTKALGISYGRKKRRQRRRAHQNSQTHQASLSKQRISSTVAAADAAVTPEERHLSKMQQNGYENPTYKFFEQMQN

SEQ ID NO: 3 - DNA, 2352 bp, human
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttataagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaagtattggtgttctttgcagaagatgtgggttcaaacaaaggtgcaatcattggactcatggtgggcggtgttgtcatagcgacagtgatcgtcatcaccttggtgatgctgaagaagaaaaagcttggtaccgagctcggatccactagtccagtgtggtggaattctgcagatatcctggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagaggatatccagcacagtggcggccgcagacgccgctgtcaccccagaggagcgccacctgtccaagatgcagcagaacggctacgaaaatccaacctacaagttctttgagcagatgcagaactag

SEQ ID NO: 4 - protein, 783 aa, fusion protein - human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFISDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQVLVFFAEDVGSNKGAIIGLMVGGVVIATVIVITLVMLKKKKLGTELGSTSPVWWNSADILEPVDPRLEPWKHPGSQPKTACTNCYCKKCCFHCQVCFMTKALGISYGRKKRRQRRRAHQNSQTHQASLSKQRISSTVAAADAAVTPEERHLSKMQQNGYENPTYKFFEQMQN

SEQ ID NO: 5 - DNA, 2823 bp, human
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttataagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaatctagatgcagaattccgacatgactcaggatatgaagttcatcatcaagtattggtgttctttgcagaagatgtgggttcaaacaaaggtgcaatcattggactcatggtgggcggtgttgtcatagcgacagtgatcgtcatcaccttggtgatgctgaagaagaaaaagcttggtaccgagctcggatccactagtccagtgtggtggaattctgcagatatcaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgggaattcccggggatctggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatatccagcacagtggcggccgcgacgccgctgtcaccccagaggagcgccacctgtccaagatgcagcagaacggctacgaaaatccaacctacaagttctttgagcagatgcagaactag

SEQ ID NO: 6 - protein, 941 aa, fusion protein - human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFISDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVNLDAEFRHDSGYEVHHQVLVFFAEDVGSNKGAIIGLMVGGVVIATVIVITLVMLKKKKLGTELGSTSPVWWNSADIKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGIPGDLAPPTDVSLGDELHLDGEDVAMAHADALDDFDLDMLGDGDSPGPGFTPHDSAPYGALDMADFEFEQMFTDALGIDEYGGDIQHSGAAADAAVTPEERHLSKMQQNGYENPTYKFFEQMQN

SEQ ID NO: 7 - DNA, 2823 bp, human
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttataagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaagtattggtgttctttgcagaagatgtgggttcaaacaaaggtgcaatcattggactcatggtgggcggtgttgtcatagcgacagtgatcgtcatcaccttggtgatgctgaagaagaaaaagcttggtaccgagctcggatccactagtccagtgtggtggaattctgcagatatcaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgggaattcccggggatctggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatatccagcacagtggcggccgcgacgccgctgtcaccccagaggagcgccacctgtccaagatgcagcagaacggctacgaaaatccaacctacaagttctttgagcagatgcagaactag

SEQ ID NO: 8 - protein, 941 aa, fusion protein - human [sequence omitted]

SEQ ID NO: 9 - DNA, 2352 bp, human
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttataagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaatctagatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaaaagcttggtaccgagctcggatccactagtccagtgtggtggaattctgcagatatcctggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagaggatatccagcacagtggcggccgcagacgccgctgtcaccccagaggagcgccacctgtccaagatgcagcagaacggctacgaaaatccaacctacaagttctttgagcagatgcagaactag

SEQ ID NO: 10 - protein, 783 aa, fusion protein - human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFISDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVNLDAEFRHDSGYEVHHQKLVFFAEDVGSNKGAIIGLMVGGVVIATVIVITLVMLKKKKLGTELGSTSPVWWNSADILEPVDPRLEPWKHPGSQPKTACTNCYCKKCCFHCQVCFMTKALGISYGRKKRRQRRRAHQNSQTHQASLSKQRISSTVAAADAAVTPEERHLSKMQQNGYENPTYKFFEQMQN

SEQ ID NO: 11 - DNA, 2352 bp, human
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttataagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaaaagcttggtaccgagctcggatccactagtccagtgtggtggaattctgcagatatcctggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagaggatatccagcacagtggcggccgcagacgccgctgtcaccccagaggagcgccacctgtccaagatgcagcagaacggctacgaaaatccaacctacaagttctttgagcagatgcagaactag

SEQ ID NO: 12 - protein, 783 aa, fusion protein - human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFISDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKKLGTELGSTSPVWWNSADILEPVDPRLEPWKHPGSQPKTACTNCYCKKCCFHCQVCFMTKALGISYGRKKRRQRRRAHQNSQTHQASLSKQRISSTVAAADAAVTPEERHLSKMQQNGYENPTYKFFEQMQN

SEQ ID NO: 13 - DNA, 2823 bp, human
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttataagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaatctagatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaaaagcttggtaccgagctcggatccactagtccagtgtggtggaattctgcagatatcaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgggaattcccggggatctggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatatccagcacagtggcggccgcgacgccgctgtcaccccagaggagcgccacctgtccaagatgcagcagaacggctacgaaaatccaacctacaagttctttgagcagatgcagaactag

SEQ ID NO: 14 - protein, 941 aa, fusion protein - human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFISDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVNLDAEFRHDSGYEVHHQKLVFFAEDVGSNKGAIIGLMVGGVVIATVIVITLVMLKKKKLGTELGSTSPVWWNSADIKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGIPGDLAPPTDVSLGDELHLDGEDVAMAHADALDDFDLDMLGDGDSPGPGFTPHDSAPYGALDMADFEFEQMFTDALGIDEYGGDIQHSGAAADAAVTPEERHLSKMQQNGYENPTYKFFEQMQN

SEQ ID NO: 15 - DNA, 2823 bp, human
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttataagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaaaagcttggtaccgagctcggatccactagtccagtgtggtggaattctgcagatatcaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgggaattcccggggatctggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatatccagcacagtggcggccgcgacgccgctgtcaccccagaggagcgccacctgtccaagatgcagcagaacggctacgaaaatccaacctacaagttctttgagcagatgcagaactag

SEQ ID NO: 16 - protein, 941 aa, fusion protein - human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFISDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKKLGTELGSTSPVWWNSADIKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGIPGDLAPPTDVSLGDELHLDGEDVAMAHADALDDFDLDMLGDGDSPGPGFTPHDSAPYGALDMADFEFEQMFTDALGIDEYGGDIQHSGAAADAAVTPEERHLSKMQQNGYENPTYKFFEQMQN

SEQ ID NO: 17 - DNA, 3354 bp, human
agtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagc

SEQ ID NO: 18 - protein, 695 aa, human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 19 - DNA, 3972 bp, human
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgaactagatctagagtccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacggaccgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacactcgacctcgaaacttgtttattgcagcttataatggttacaaataaagcaatagcatcacaaatttcacaaataaagcatttttttcactgcattctagttgtggtttgtccaaactcatcaatgtatcttatcatgtctggatcgatacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttggtaccgagctcggatccgaattcgccacc

SEQ ID NO: 20 - DNA, 3972 bp, human
taccactcgttcccgctcctcgacaagtggccccaccacgggtaggaccagctcgacctgccgctgcatttgccggtgttcaagtcgcacaggccgctcccgctcccgctacggtggatgccgttcgactgggacttcaagtagacgtggtggccgttcgacgggcacgggaccgggtgggagcactggtggaagtggatgccgcacgtcacgaagcgggcgatggggctggtgtacttcgtcgtgctgaagaagttcaggcggtacgggcttccgatgcaggtcctcgcgtggtagaagaagttcctgctgccgttgatgttctgggcgcggctccacttcaagctcccgctgtgggaccacttggcgtagctcgacttcccgtagctgaagttcctcctgccgttgtaggaccccgtgttcgacctcatgttgatgttgtcggtgttccagatatagtggcggctgttcgtcttcttgccgtagttccacttgaagttctgggcggtgttgtagctcctgccgtcgcacgtcgagcggctggtgatggtcgtcttgtgggggtagccgctgccggggcacgacgacgggctgttggtgatggactcgtgggtcaggcgggactcgtttctggggttgctcttcgcgctagtgtaccaggacgacctcaagcactggcggcggccctagtgagagccgtacctgctcgacatgttcattgagctcagatctcccgggcaaatttgggcgactagtcggagctgacacggaagatcaacggtcggtagacaacaaacggggagggggcacggaaggaactgggaccttccacggtgagggtgacaggaaaggattattttactcctttaacgtagcgtaacagactcatccacagtaagataagaccccccaccccaccccgtcctgtcgttccccctcctaacccttctgttatcgtccgtacgacccctacgccacccgagataccgaagactccgcctttcttggtcgtacactcgttttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcttgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactgtaattggatatttttatccgcatagtgctccgggaaagcagagcgcgcaaagccactactgccacttttggagactgtgtacgtcgagggcctctgccagtgtcgaacagacattcgcctacggccctcgtctgttcgggcagtcccgcgcagtcgcccacaaccgcccacagccccgaccgaattgatacgccgtagtctcgtctaacatgactctcacgtggtatacgccacactttatggcgtgtctacgcattcctcttttatggcgtagtcctgcgcgggacatcgccgcgtaattcgcgccgcccacaccaccaatgcgcgtcgcactggcgatgtgaacggtcgcgggatcgcgggcgaggaaagcgaaagaagggaaggaaagagcggtgcaagcggccgaaaggggcagttcgagatttagcccccgagggaaatcccaaggctaaatcacgaaatgccgtggagctggggttttttgaactaatcccactaccaagtgcatcacccggtagcgggactatctgccaaaaagcgggaaactgcaacctcaggtgcaagaaattatcacctgagaacaaggtttgaccttgttgtgagttgggatagagccagataagaaaactaaatattccctaaaacggctaaagccggataaccaattttttactcgactaaattgtttttaaattgcgcttaaaattgttttataattgcgaatgttaaaggtaagcggtaagtccgacttgatctagatctcaggcaatgtattgaatgccatttaccgggcggaccgactggcgggttgctgggggcgggtaactgcagttattactgcatacaagggtatcattgcggttatccctgaaaggtaactgcagttacccacctcataaatgccatttgacgggtgaaccgtcatgtagttcacatagtatacggttcatgcgggggataactgcagttactgccatttaccgggcggaccgtaatacgggtcatgtactggaataccctgaaaggatgaaccgtcatgtagatgcataatcagtagcgataatggtaccactacgccaaaaccgtcatgtagttacccgcacctatcgccaaactgagtgcccctaaaggttcagaggtggggtaactgcagttaccctcaaacaaaaccgtggttttagttgccctgaaaggttttacagcattgttgaggcggggtaactgcgtttacccgccatccgcacatgccaccctccagatatattcgtctcgagcaaatcacttggcagtctagcggacctctgcggtaggtgcgacaaaactggaggtatcttctgtggccctggctaggtcggaggcgccggcccttgccacgtaaccttgcctggcacaactgttaattagtagccgtatcatatagccgtatcatattatgctgttccactccttgatttggtaccggttcaactggtcacggcaaggccacgagtggcgcgcgctgcagcggcctcgccagctcaagacctggctggccgagcccaagagggccctgaagcacctcctgctgaagcggccacaccaggccctgctgcactgggacaagtagtcgcgccaggtcctggtccaccacggcctgttgtgggaccggacccacacccacgcgccggacctgctcgacatgcggctcaccagcctccagcacaggtgcttgaaggccctgcggaggcccggccggtactggctctagccgctcgtcggcacccccgccctcaagcgggacgcgctgggccggccgttgacgcacgtgaagcaccggctcctcgtcctgactgtgagctggagctttgaacaaataacgtcgaatattaccaatgtttatttcgttatcgtagtgtttaaagtgtttatttcgtaaaaaaagtgacgtaagatcaacaccaaacaggtttgagtagttacatagaatagtacagacctagctatgaagttcttgacgactgtagctcgaacgatgttccctgaaaggcgacccctgaaaggtccctccgcaccggacccgccctgacccctcaccgctcgggagtctaggacgtatattcgtcgacgaaaaacggacatgacccagagagaccaatctggtctagactcggaccctcgagagaccgattgatcccttgggtgacgaattcggagttatttcgaaccatggctcgagcctaggcttaagcggtgg

SEQ ID NO: 21 - DNA, 7153 bp, human
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttataagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaatctagatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaagatatcatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 22 - DNA, 7153 bp, human
ctgcctagccctctagagggctaggggataccagctgagagtcatgttagacgagactacggcgtatcaattcggtcatagacgagggacgaacacacaacctccagcgactcatcacgcgctcgttttaaattcgatgttgttccgttccgaactggctgttaacgtacttcttagacgaatcccaatccgcaaaacgcgacgaagcgctacatgcccggtctatatgcgcaactgtaactaataactgatcaataattatcattagttaatgccccagtaatcaagtatcgggtatatacctcaaggcgcaatgtattgaatgccatttaccgggcggaccgactggcgggttgctgggggcgggtaactgcagttattactgcatacaagggtatcattgcggttatccctgaaaggtaactgcagttacccacctgataaatgccatttgacgggtgaaccgtcatgtagttcacatagtatacggttcatgcgggggataactgcagttactgccatttaccgggcggaccgtaatacgggtcatgtactggaataccctgaaaggatgaaccgtcatgtagatgcataatcagtagcgataatggtaccactacgccaaaaccgtcatgtagttacccgcacctatcgccaaactgagtgcccctaaaggttcagaggtggggtaactgcagttaccctcaaacaaaaccgtggttttagttgccctgaaaggttttacagcattgttgaggcggggtaactgcgtttacccgccatccgcacatgccaccctccagatatattcgtctcgagagaccgattgatctcttgggtgacgaatgaccgaatagctttaattatgctgagtgatatccctctgggttcgaccgatcgcaaatttgaattcgaaggggcgcgtcccagcgctacgacgggccaaaccgtgacgaggacgaccggcggacctgccgagcccgcgacctccatgggtgactaccattacgaccggacgaccgacttggggtctaacggtacaagacaccgtctgacttgtacgtgtacttacaggtcttacccttcaccctaagtctaggtagtccctggttttggacgtaactatggttccttccgtaggacgtcataacggttcttcagatgggacttgacgtctagtggttacaccatcttcggttggttggtcactggtaggtcttgaccacgttcgccccggcgttcgtcacgttctgggtaggggtgaaacactaagggatggcgacgaatcaaccactcaaatattcactacgggaagagcaaggactgttcacgtttaagaatgtggtcctctcctacctacaaacgctttgagtagaagtgaccgtgtggcagcggtttctctgtacgtcactcttctcatggttgaacgtactgatgccgtacaacgacgggacgccttaactgttcaaggctccccatctcaaacacacaacgggtgaccgacttctttcactgttacacctaagacgactacgcctcctcctactgagcctacagaccaccccgcctcgtctgtgtctgatacgtctaccctcacttctgtttcatcatcttcatcgtctcctccttcttcaccgactccaccttcttcttcttcggctactactgctcctgctactcctaccactactccatctccttctccgactccttgggatgcttcttcggtgtctctcttggtggtcgtaacggtggtggtggtggtggtggtgtctcagacaccttctccaccaagctcaaggatgttgtcgtcggtcatggggactacggcaactgttcatagagctctgtggacccctactcttacttgtacgggtaaaggtctttcggtttctctccgaactccggttcgtggctctctcttacagggtccagtactctcttacccttctccgtcttgcagttcgtttcttgaacggatttcgactattcttccgtcaataggtcgtaaaggtcctctttcaccttagaaaccttgtccttcgtcggttgctctctgtcgtcgaccacctctgtgtgtaccggtctcaccttcggtacgagttactggcggcggcggaccgggacctcttgatgtagtggcgagacgtccgacaaggaggagccggagcagtgcacaagttatacgatttcttcatacaggcgcgtcttgtcttcctgtctgtcgtgtgggatttcgtaaagctcgtacacgcgtaccacctagggttctttcggcgagtctaggccagggtccaatactgtgtggaggcacactaaatactcgcgtacttagtcagagagagggacgagatgttgcacggacgtcaccggctcctctaagtcctacttcaactactcgacgaagtctttctcgttttgataagtctactgcagaaccggttgtactaatcacttggttcctagtcaatgcctttgctacgagagtacggtagaaactggctttgcttttggtggcacctcgaggaagggcacttacctctcaagtcggacctgctagaggtcggcaccgtaagaaaaccccgactgagacacggtcggttgtgtcttttgcttcaactcggacaactacgggcgggacgacggctggctcctgactggtgagctggtccaagacccaactgtttatagttctgcctcctctagagacttcacttagatctacgtcttaaggctgtactgagtcctatacttcaagtagtagtttttaaccacaagaaacgtcttctacacccaagtttgtttccacgttagtaacctgagtaccacccgccacaacagtatcgctgtcactagcagtagtggaaccactacgacttcttctttctatagtacctcggtcatctaggatctgatctcgggaccttcgtaggtccttcagtcggattttgacgaacatggttaacgataacatttttcacaacgaaagtaacggttcaaacaaagtactgttttcggaatccgtagaggataccgtccttcttcgcctctgtcgctgcttctcgagtagtcttgtcagtctgagtagttcgaagagatagtttcgtcattcatccgccggcgagctcagatctcccgggcaaatttgggcgactagtcggagctgacacggaagatcaacggtcggtagacaacaaacggggagggggcacggaaggaactgggaccttccacggtgagggtgacaggaaaggattattttactcctttaacgtagcgtaacagactcatccacagtaagataagaccccccaccccaccccgtcctgtcgttccccctcctaacccttctgttatcgtccgtacgacccctacgccacccgagataccgaagactccgcctttcttggtcgaccccgagatcccccataggggtgcgcgggacatcgccgcgtaattcgcgccgcccacaccaccaatgcgcgtcgcactggcgatgtgaacggtcgcgggatcgcgggcgaggaaagcgaaagaagggaaggaaagagcggtgcaagcggccgaaaggggcagttcgagatttagccccgtagggaaatcccaaggctaaatcacgaaatgccgtggagctggggttttttgaactaatcccactaccaagtgcatcacccggtagcgggactatctgccaaaaagcgggaaactgcaacctcaggtgcaagaaattatcacctgagaacaaggtttgaccttgttgtgagttgggatagagccagataagaaaactaaatattccctaaaacccctaaagccggataaccaattttttactcgactaaattgtttttaaattgcgcttaattaagacaccttacacacagtcaatcccacacctttcaggggtccgaggggtccgtccgtcttcatacgtttcgtacgtagagttaatcagtcgttggtccacacctttcaggggtccgaggggtcgtccgtcttcatacgtttcgtacgtagagttaatcagtcgttggtatcagggcggggattgaggcgggtagggcggggattgaggcgggtcaaggcgggtaagaggcggggtaccgactgattaaaaaaaataaatacgtctccggctccggcggagacggagactcgataaggtcttcatcactcctccgaaaaaacctccggatccgaaaacgtttttcgagggccctcgaacatataggtaaaagcctagactagtcgtgcacaactgttaattagtagccgtatcatatagccgtatcatattatgctgttccactccttgatttggtaccggttcaactggtcacggcaaggccacgagtggcgcgcgctgcagcggcctcgccagctcaagacctggctggccgagcccaagagggccctgaagcacctcctgctgaagcggccacaccaggccctgctgcactgggacaagtagtcgcgccaggtcctggtccaccacggcctgttgtgggaccggacccacacccacgcgccggacctgctcgacatgcggctcaccagcctccagcacaggtgcttgaaggccctgcggaggcccggccggtactggctctagccgctcgtcggcacccccgccctcaagcgggacgcgctgggccggccgttgacgcacgtgaagcaccggctcctcgtcctgactgtgcacgatgctctaaagctaaggtggcggcggaagatactttccaacccgaagccttagcaaaaggccctgcggccgacctactaggaggtcgcgcccctagagtacgacctcaagaagcgggtggggttgaacaaataacgtcgaatattaccaatgtttatttcgttatcgtagtgtttaaagtgtttatttcgtaaaaaaagtgacgtaagatcaacaccaaacaggtttgagtagttacatagaatagtacagacatatggcagctggagatcgatctcgaaccgcattagtaccagtatcgacaaaggacacactttaacaataggcgagtgttaaggtgtgttgtatgctcggccttcgtatttcacatttcggaccccacggattactcactcgattgagtgtaattaacgcaacgcgagtgacgggcgaaaggtcagccctttggacagcacggtcgacgtaattacttagccggttgcgcgcccctctccgccaaacgcataacccgcgagaaggcgaaggagcgagtgactgagcgacgcgagccagcaagccgacgccgctcgccatagtcgagtgagtttccgccattatgccaataggtgtcttagtcccctattgcgtcctttcttgtacactcgttttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagttacgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcctgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttaatttttacttcaaaatttagttagatttcatatatactcatttgaaccagactgtcaatggttacgaattagtcactccgtggatagagtcgctagacagataaagcaagtaggtatcaacggactgaggggcagcacatctattgatgctatgccctcccgaatggtagaccggggtcacgacgttactatggcgctctgggtgcgagtggccgaggtctaaatagtcgttatttggtcggtcggccttcccggctcgcgtcttcaccaggacgttgaaataggcggaggtaggtcagataattaacaacggcccttcgatctcattcatcaagcggtcaattatcaaacgcgttgcaacaacggtaacgatgtccgtagcaccacagtgcgagcagcaaaccataccgaagtaagtcgaggccaagggttgctagttccgctcaatgtactagggggtacaacacgttttttcgccaatcgaggaagccaggaggctagcaacagtcttcattcaaccggcgtcacaatagtgagtaccaataccgtcgtgacgtattaagagaatgacagtacggtaggcattctacgaaaagacactgaccactcatgagttggttcagtaagactcttatcacatacgccgctggctcaacgagaacgggccgcagttatgccctattatggcgcggtgtatcgtcttgaaattttcacgagtagtaaccttttgcaagaagccccgcttttgagagttcctagaatggcgacaactctaggtcaagctacattgggtgagcacgtgggttgactagaagtcgtagaaaatgaaagtggtcgcaaagacccactcgtttttgtccttccgttttacggcgttttttcccttattcccgctgtgcctttacaacttatgagtatgagaaggaaaaagttataataacttcgtaaatagtcccaataacagagtactcgcctatgtataaacttacataaatctttttatttgtttatccccaaggcgcgtgtaaaggggcttttcacggtggactgcag

SEQ ID NO: 23 - DNA, 2352 bp, human
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttataagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaactttgaagtggaattccgacatgactcaggatatgaagttcatcatcaagtattggtgttctttgcagaagatgtgggttcaaacaaaggtgcaatcattggactcatggtgggcggtgttgtcatagcgacagtgatcgtcatcaccttggtgatgctgaagaagaaaaagcttggtaccgagctcggatccactagtccagtgtggtggaattctgcagatatcctggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagaggatatccagcacagtggcggccgcagacgccgctgtcaccccagaggagcgccacctgtccaagatgcagcagaacggctacgaaaatccaacctacaagttctttgagcagatgcagaactag

SEQ ID NO: 24 - protein, 783 aa, fusion protein - human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFISDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVNFEVEFRHDSGYEVHHQVLVFFAEDVGSNKGAIIGLMVGGVVIATVIVITLVMLKKKKLGTELGSTSPVWWNSADILEPVDPRLEPWKHPGSQPKTACTNCYCKKCCFHCQVCFMTKALGISYGRKKRRQRRRAHQNSQTHQASLSKQRISSTVAAADAAVTPEERHLSKMQQNGYENPTYKFFEQMQN

SEQ ID NO: 25 - DNA, 2823 bp, human
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttataagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaactttgaagtggaattccgacatgactcaggatatgaagttcatcatcaagtattggtgttctttgcagaagatgtgggttcaaacaaaggtgcaatcattggactcatggtgggcggtgttgtcatagcgacagtgatcgtcatcaccttggtgatgctgaagaagaaaaagcttggtaccgagctcggatccactagtccagtgtggtggaattctgcagatatcaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgggaattcccggggatctggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatatccagcacagtggcggccgcgacgccgctgtcaccccagaggagcgccacctgtccaagatgcagcagaacggctacgaaaatccaacctacaagttctttgagcagatgcagaactag

SEQ ID NO: 26 - protein, 941 aa, fusion protein - human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFISDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVNFEVEFRHDSGYEVHHQVLVFFAEDVGSNKGAIIGLMVGGVVIATVIVITLVMLKKKKLGTELGSTSPVWWNSADIKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGIPGDLAPPTDVSLGDELHLDGEDVAMAHADALDDFDLDMLGDGDSPGPGFTPHDSAPYGALDMADFEFEQMFTDALGIDEYGGDIQHSGAAADAAVTPEERHLSKMQQNGYENPTYKFFEQMQN

SEQ ID NO: 27 - DNA, 5015 bp, human
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 28 - DNA, 5015 bp, human
ctgcctagccctctagagggctaggggataccagctgagagtcatgttagacgagactacggcgtatcaattcggtcatagacgagggacgaacacacaacctccagcgactcatcacgcgctcgttttaaattcgatgttgttccgttccgaactggctgttaacgtacttcttagacgaatcccaatccgcaaaacgcgacgaagcgctacatgcccggtctatatgcgcaactgtaactaataactgatcaataattatcattagttaatgccccagtaatcaagtatcgggtatatacctcaaggcgcaatgtattgaatgccatttaccgggcggaccgactggcgggttgctgggggcgggtaactgcagttattactgcatacaagggtatcattgcggttatccctgaaaggtaactgcagttacccacctgataaatgccatttgacgggtgaaccgtcatgtagttcacatagtatacggttcatgcgggggataactgcagttactgccatttaccgggcggaccgtaatacgggtcatgtactggaataccctgaaaggatgaaccgtcatgtagatgcataatcagtagcgataatggtaccactacgccaaaaccgtcatgtagttacccgcacctatcgccaaactgagtgcccctaaaggttcagaggtggggtaactgcagttaccctcaaacaaaaccgtggttttagttgccctgaaaggttttacagcattgttgaggcggggtaactgcgtttacccgccatccgcacatgccaccctccagatatattcgtctcgagagaccgattgatctcttgggtgacgaatgaccgaatagctttaattatgctgagtgatatccctctgggttcgaccgatcgcaaatttgaattcgaaccatggctcgagcctaggtgatcaggtcacaccaccttaagacgtctataggtcgtgtcaccgccggcgagctcagatctcccgggcaaatttgggcgactagtcggagctgacacggaagatcaacggtcggtagacaacaaacggggagggggcacggaaggaactgggaccttccacggtgagggtgacaggaaaggattattttactcctttaacgtagcgtaacagactcatccacagtaagataagaccccccaccccaccccgtcctgtcgttccccctcctaacccttctgttatcgtccgtacgacccctacgccacccgagataccgaagactccgcctttcttggtcgaccccgagatcccccataggggtgcgcgggacatcgccgcgtaattcgcgccgcccacaccaccaatgcgcgtcgcactggcgatgtgaacggtcgcgggatcgcgggcgaggaaagcgaaagaagggaaggaaagagcggtgcaagcggccgaaaggggcagttcgagatttagccccgtagggaaatcccaaggctaaatcacgaaatgccgtggagctggggttttttgaactaatcccactaccaagtgcatcacccggtagcgggactatctgccaaaaagcgggaaactgcaacctcaggtgcaagaaattatcacctgagaacaaggtttgaccttgttgtgagttgggatagagccagataagaaaactaaatattccctaaaacccctaaagccggataaccaattttttactcgactaaattgtttttaaattgcgcttaattaagacaccttacacacagtcaatcccacacctttcaggggtccgaggggtccgtccgtcttcatacgtttcgtacgtagagttaatcagtcgttggtccacacctttcaggggtccgaggggtcgtccgtcttcatacgtttcgtacgtagagttaatcagtcgttggtatcagggcggggattgaggcgggtagggcggggattgaggcgggtcaaggcgggtaagaggcggggtaccgactgattaaaaaaaataaatacgtctccggctccggcggagacggagactcgataaggtcttcatcactcctccgaaaaaacctccggatccgaaaacgtttttcgagggccctcgaacatataggtaaaagcctagactagtcgtgcacaactgttaattagtagccgtatcatatagccgtatcatattatgctgttccactccttgatttggtaccggttcaactggtcacggcaaggccacgagtggcgcgcgctgcagcggcctcgccagctcaagacctggctggccgagcccaagagggccctgaagcacctcctgctgaagcggccacaccaggccctgctgcactgggacaagtagtcgcgccaggtcctggtccaccacggcctgttgtgggaccggacccacacccacgcgccggacctgctcgacatgcggctcaccagcctccagcacaggtgcttgaaggccctgcggaggcccggccggtactggctctagccgctcgtcggcacccccgccctcaagcgggacgcgctgggccggccgttgacgcacgtgaagcaccggctcctcgtcctgactgtgcacgatgctctaaagctaaggtggcggcggaagatactttccaacccgaagccttagcaaaaggccctgcggccgacctactaggaggtcgcgcccctagagtacgacctcaagaagcgggtggggttgaacaaataacgtcgaatattaccaatgtttatttcgttatcgtagtgtttaaagtgtttatttcgtaaaaaaagtgacgtaagatcaacaccaaacaggtttgagtagttacatagaatagtacagacatatggcagctggagatcgatctcgaaccgcattagtaccagtatcgacaaaggacacactttaacaataggcgagtgttaaggtgtgttgtatgctcggccttcgtatttcacatttcggaccccacggattactcactcgattgagtgtaattaacgcaacgcgagtgacgggcgaaaggtcagccctttggacagcacggtcgacgtaattacttagccggttgcgcgcccctctccgccaaacgcataacccgcgagaaggcgaaggagcgagtgactgagcgacgcgagccagcaagccgacgccgctcgccatagtcgagtgagtttccgccattatgccaataggtgtcttagtcccctattgcgtcctttcttgtacactcgttttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagttacgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcctgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttaatttttacttcaaaatttagttagatttcatatatactcatttgaaccagactgtcaatggttacgaattagtcactccgtggatagagtcgctagacagataaagcaagtaggtatcaacggactgaggggcagcacatctattgatgctatgccctcccgaatggtagaccggggtcacgacgttactatggcgctctgggtgcgagtggccgaggtctaaatagtcgttatttggtcggtcggccttcccggctcgcgtcttcaccaggacgttgaaataggcggaggtaggtcagataattaacaacggcccttcgatctcattcatcaagcggtcaattatcaaacgcgttgcaacaacggtaacgatgtccgtagcaccacagtgcgagcagcaaaccataccgaagtaagtcgaggccaagggttgctagttccgctcaatgtactagggggtacaacacgttttttcgccaatcgaggaagccaggaggctagcaacagtcttcattcaaccggcgtcacaatagtgagtaccaataccgtcgtgacgtattaagagaatgacagtacggtaggcattctacgaaaagacactgaccactcatgagttggttcagtaagactcttatcacatacgccgctggctcaacgagaacgggccgcagttatgccctattatggcgcggtgtatcgtcttgaaattttcacgagtagtaaccttttgcaagaagccccgcttttgagagttcctagaatggcgacaactctaggtcaagctacattgggtgagcacgtgggttgactagaagtcgtagaaaatgaaagtggtcgcaaagacccactcgtttttgtccttccgttttacggcgttttttcccttattcccgctgtgcctttacaacttatgagtatgagaaggaaaaagttataataacttcgtaaatagtcccaataacagagtactcgcctatgtataaacttacataaatctttttatttgtttatccccaaggcgcgtgtaaaggggcttttcacggtggactgcag

SEQ ID NO: 29 - DNA, 2352 bp, human
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttataagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaactttgaagtggaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaaaagcttggtaccgagctcggatccactagtccagtgtggtggaattctgcagatatcctggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagaggatatccagcacagtggcggccgcagacgccgctgtcaccccagaggagcgccacctgtccaagatgcagcagaacggctacgaaaatccaacctacaagttctttgagcagatgcagaactag

SEQ ID NO: 30 - protein, 783 aa, fusion protein - human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFISDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVNFEVEFRHDSGYEVHHQKLVFFAEDVGSNKGAIIGLMVGGVVIATVIVITLVMLKKKKLGTELGSTSPVWWNSADILEPVDPRLEPWKHPGSQPKTACTNCYCKKCCFHCQVCFMTKALGISYGRKKRRQRRRAHQNSQTHQASLSKQRISSTVAAADAAVTPEERHLSKMQQNGYENPTYKFFEQMQN

SEQ ID NO: 31 - DNA, 2823 bp, human
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttataagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaactttgaagtggaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaaaagcttggtaccgagctcggatccactagtccagtgtggtggaattctgcagatatcaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgggaattcccggggatctggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatatccagcacagtggcggccgcgacgccgctgtcaccccagaggagcgccacctgtccaagatgcagcagaacggctacgaaaatccaacctacaagttctttgagcagatgcagaactag

SEQ ID NO: 32 - protein, 941 aa, fusion protein - human
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFISDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVNFEVEFRHDSGYEVHHQKLVFFAEDVGSNKGAIIGLMVGGVVIATVIVITLVMLKKKKLGTELGSTSPVWWNSADIKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGIPGDLAPPTDVSLGDELHLDGEDVAMAHADALDDFDLDMLGDGDSPGPGFTPHDSAPYGALDMADFEFEQMFTDALGIDEYGGDIQHSGAAADAAVTPEERHLSKMQQNGYENPTYKFFEQMQN

SEQ ID NO: 33 - protein, 63 aa, human
EEISEVKMDAEFRHDSGYEVHHQKLVFFAEDVGSNKGAIIGLMVGGVVIATVIVITLVMLKKK

SEQ ID NO: 34 - protein, 4 aa, human
KMDA

SEQ ID NO: 35 - protein, 7 aa, human
GVVIATV

SEQ ID NO: 36 - protein, 63 aa, human
EEISEVKMDAEFRHDSGYEVHHQVLVFFAEDVGSNKGAIIGLMVGGVVIATVIVITLVMLKKK

SEQ ID NO: 37 - protein, 63 aa, human
EEISEVNLDAEFRHDSGYEVHHQVLVFFAEDVGSNKGAIIGLMVGGVVIATVIVITLVMLKKK

SEQ ID NO: 38 - protein, 4 aa, human
NLDA

SEQ ID NO: 39 - protein, 63 aa, human
EEISEVNFEVEFRHDSGYEVHHQVLVFFAEDVGSNKGAIIGLMVGGVVIATVIVITLVMLKKK

SEQ ID NO: 40 - protein, 4 aa, human
NFEV

SEQ ID NO: 41 - protein, 5 aa, human
VNFAV

SEQ ID NO: 42 - protein, 5 aa, human
VKVDA

SEQ ID NO: 43 - protein, 5 aa, human
WKMDA

SEQ ID NO: 44 - protein, 5 aa, human
VKADA

SEQ ID NO: 45 - protein, 5 aa, human
VKKDA

SEQ ID NO: 46 - protein, 5 aa, human
VKEDA

SEQ ID NO: 47 - protein, 5 aa, human
VKIDA

SEQ ID NO: 48 - protein, 5 aa, human
VKMIA

SEQ ID NO: 49 - protein, 5 aa, human
VKMNA

SEQ ID NO: 50 - protein, 5 aa, human
VKMEA

SEQ ID NO: 51 - protein, 5 aa, human
VKMDE

SEQ ID NO: 52 - protein, 5 aa, human
VKMDK

SEQ ID NO: 53 - DNA, 27 bp, Artificial Sequence
  PCR primer
ggagaggatatcatggagccagtagat

SEQ ID NO: 54 - DNA, 29 bp, Artificial Sequence
  PCR primer
tacatggcggccgcctacttactgctttg

SEQ ID NO: 55 - DNA, 35 bp, Artificial Sequence
  PCR primer
ggatgtgatatctttcttcttcagcatcaccaagg

SEQ ID NO: 56 - DNA, 36 bp, Artificial Sequence
  PCR Primer
ctgagatatcaagctactgtcttctatcgaacaagc

SEQ ID NO: 57 - DNA, 31 bp, Artificial Sequence
  PCR Primer
gcgcgatatccccaccgtactcgtcaattcc

SEQ ID NO: 58 - DNA, 34 bp, Artificial Sequence
  PCR Primer
ctgctgtggcggccgcctagttctgcatctgctc

SEQ ID NO: 59 - DNA, 30 bp, Artificial Sequence
  PCR Primer
tgccccgcgcggccgcgcgatgctgcccgg

SEQ ID NO: 60 - DNA, 32 bp, Artificial Sequence
  PCR Primer
atggtgtggcggccgcagacgccgctgtcacc

SEQ ID NO: 61 - DNA, 36 bp, Artificial Sequence
  PCR Primer
agcgcacaagcttccccgcgcagggtcgcgatgctg

SEQ ID NO: 62 - DNA, 35 bp, Artificial Sequence
  PCR Primer
ggatgtaagctttttcttcttcagcatcaccaagg

SEQ ID NO: 63 - DNA, 32 bp, Artificial Sequence
  PCR Primer
tgcagatatcctggagccagtagatcctagac

SEQ ID NO: 64 - DNA, 30 bp, Artificial Sequence
  PCR Primer
gctggatatcctctgctttgatagagaagc